SEQUENCE LISTING (18 sequences)

SEQ ID NO: 1 - DNA, 16 bp, Artificial Sequence
  synthesized oligonucleotide
tcaccacatcccagtg

SEQ ID NO: 2 - DNA, 16 bp, Artificial Sequence
  synthesized oligonucleotide
gagggaggtttggctg

SEQ ID NO: 3 - DNA, 22 bp, Artificial Sequence
  synthesized oligonucleotide
ccagcaaccaatgatgcccgtt

SEQ ID NO: 4 - DNA, 22 bp, Artificial Sequence
  synthesized oligonucleotide
ccagcaagcactgatgcctgtt

SEQ ID NO: 5 - protein, 4 aa, Artificial Sequence
  peptide linker
KKAA

SEQ ID NO: 6 - protein, 4 aa, Artificial Sequence
  peptide linker
KKKA

SEQ ID NO: 7 - protein, 4 aa, Artificial Sequence
  peptide linker
KKKK

SEQ ID NO: 8 - DNA, 25 bp, Artificial Sequence
  synthesized oligonucleotide
gaccaggaaatagagaggaaatgta

SEQ ID NO: 9 - DNA, 27 bp, Artificial Sequence
  synthesized oligonucleotide
gaaggagaaggaagagttggtattatc

SEQ ID NO: 10 - DNA, 21 bp, Artificial Sequence
  synthesized oligonucleotide
ttgggctcagatctgtgatag

SEQ ID NO: 11 - DNA, 27 bp, Artificial Sequence
  synthesized oligonucleotide
catctaggtatccaaaaggagagtcta

SEQ ID NO: 12 - DNA, 27 bp, Artificial Sequence
  synthesized oligonucleotide
cggtatatagttcttcctcatgctatt

SEQ ID NO: 13 - DNA, 20 bp, Artificial Sequence
  synthesized oligonucleotide
gcaagatcttcgccttactg

SEQ ID NO: 14 - DNA, 32 bp, Artificial Sequence
  probe
ttccattttctttttagagcagtatacaaaga

SEQ ID NO: 15 - DNA, 32 bp, Artificial Sequence
  probe
tctttgtatactgctctaaaaagaaaatggaa

SEQ ID NO: 16 - DNA, 28 bp, Artificial Sequence
  probe
aaactccagcatagatgtggatagcttg

SEQ ID NO: 17 - DNA, 28 bp, Artificial Sequence
  probe
caagctatccacatctatgctggagttt

SEQ ID NO: 18 - DNA, 23 bp, Artificial Sequence
  probe
aactgcttgtggccatggcttag

(19) United States
(12) Patent Application Publication
(10) Pub. No.: US 2005/0053939 A1
(43) Pub. Date: Mar. 10, 2005

(54) Methods and compositions for enhancing detection in determinations employing cleavable electrophoretic tag reagents

(76) Inventors: Ahmed Chenna, Sunnyvale, CA (US); Sharat Singh, Los Altos, CA (US)

Correspondence Address: ACLARA BIOSCIENCES, INC., 1288 PEAR AVENUE, MOUNTAIN VIEW, CA, 94043, US

(21) Appl. No.: 10/494,879

(22) PCT Filed: Nov. 8, 2002
(86) PCT No.: PCT/US02/35893

Related U.S. Application Data
(60) Provisional application No. 60/337,982, filed on Nov. 9, 2001.

(30) Foreign Application Priority Data
Nov. 9, 2001 (US) 10/011201

(51) Int. Cl. C12Q 1/68; G01N 33/53

(52) U.S. Cl. 435/6; 435/7.1

(57) ABSTRACT

Probe sets for the multiplexed detection of the binding of, or interaction between, one or more ligands and target antiligands are provided. Detection involves the release of identifying tags as a consequence of target recognition. The probe sets include electrophoretic tag probes or e-tag probes, comprising a detection region and a mobility-defining region, both linked to a target-binding moiety. In a multiplexed assay, different released e-tag reporters may be separated and detected providing for target identification. The probes comprise interactive functionalities adjacent the cleaved portion positioned in the probes such that the interactive functionality does not form part of the e-tag reporters. Also described are biopolymers and nucleosides containing such interactive functionalities.

### FIELD OF THE INVENTION

The present invention relates to separable compositions, methods, and kits for use in multiplexed assay detection of the interaction between ligands and target antiligands. The methods and compositions of the invention find application to the area of purification of mixtures comprising molecules of interest, to separate unwanted materials from the molecules of interest. The invention has particular use in multiplexed assays for polypeptides and polynucleotides in which separable electrophoretic tag reagents are employed.

### BACKGROUND OF THE INVENTION

The need to determine many analytes or nucleic acid sequences (for example multiple pathogens or multiple genes or multiple genetic variants) in blood or other biological fluids has become increasingly apparent in many branches of medicine. Most multi-analyte assays, such as assays that detect multiple nucleic acid sequences, involve multiple steps, have poor sensitivity, a limited dynamic range (typically on the order of 2 to 100-fold differences and some require sophisticated instrumentation. Some of the known classical methods for multianalyte assays include the following:
(a) The use of two different radioisotope labels to distinguish two different analytes.
(b) The use of two or more different fluorescent labels to distinguish two or more analytes.
(c) The use of lanthanide chelates where both lifetime and wavelength are used to distinguish two or more analytes.
(d) The use of fluorescent and chemiluminescent labels to distinguish two or more analytes.
(e) The use of two different enzymes to distinguish two or more analytes.
(f) The use of enzyme and acridinium esters to distinguish two or more analytes.
(g) Spatial resolution of different analytes, for example on arrays, to identify and quantity multiple analytes.
(h) The use of acridinium ester labels where lifetime or dioxetanone formation is used to quantify two different viral targets.

As the human genome is elucidated, there will be numerous opportunities for performing assays to determine the presence of specific sequences, e.g. sequences associated with genes, regulatory sequences, repeats, multimeric regions, expression patterns, and the like; to distinguish between alleles in homozygotes and heterozygotes, to determine the presence of mutations, to evaluate cellular expression patterns, etc. The need to identify and quantify a large number of bases or sequences, potentially distributed over centimorgans of DNA, offers a major challenge. Determination of the presence of pathogens, antibiotic resistance genes, and genetic subtypes is of interest, as well as the analysis of differential expression of multiple genes to determine toxicologically relevant outcomes, and screening of transfused blood for viral contaminants with high sensitivity.

In many of these cases one will wish to determine, in a single reaction, a number of different characteristics of the same sample. Preferred assay methods will be accurate, highly sensitive, and reasonably economical, have a large dynamic range (e.g. 10³ to 10⁴-fold differences in target levels), and be adaptable to a highly multiplexed format, which allows for differentiation and quantitation of multiple genes, SNP determination, and/or analysis of gene expression at the RNA or protein level. Other types of compounds, such as proteins in proteomics, also offer opportunities for multiplexed determinations.

By single nucleotide polymorphism (SNPs) is intended that, at a given site in the genome of an organism, there will be a prevalent nucleotide in most of the population, with one or more of the remaining bases being present in a substantially smaller percent of the population. It is believed that one polymorphism occurs per about 1,000 bases. While other genetic markers are available, the large number of SNPs and their extensive distribution in the chromosomes make SNPs an attractive target. Also, by determining a plurality of SNPs associated with a specific phenotype, one may use the SNP pattern as an indication of the phenotype, rather than requiring a determination of the genes associated with the phenotype. For the most part, the SNPs are in non-coding regions, primarily between genes, but are also present in exons and introns. In addition, the majority of SNPs affect the genotype but not the phenotype of the individual. Since SNPs are inherited, individual SNPs and/or SNP patterns may be related to genetic defects, such as deletions, insertions and mutations, involving one or more bases in genes. Rather than isolating and sequencing the target gene, it will be sufficient to identify the SNPs involved. In addition, SNPs may be used in forensic medicine to identify individuals.

### BRIEF DESCRIPTION OF THE RELATED ART

Holland (Proc. Natl. Acad. Sci. USA (1991) 88:7276) discloses exonuclease activity of the thermostable enzyme Thermus aquaticus DNA polymerase in PCR amplification, to generate specific detectable signal concomitantly with amplification.

The TaqMan® assay is discussed by Lee in Nucleic Acid Research (1993) 21:16 3761).

White (Trends Biotechnology (1996) 14(12): 478-483) discusses the problems of multiplexing in the TaqMan assay.

Marino, Electrophoresis (1996) 17:1499 describes low-stringency sequence specific PCR (LSSP-PCR). A PCR amplified sequence is subjected to single primer amplification under conditions of low stringency to produce a range of different length amplicons. Different patterns are obtained when there are differences in sequence. The patterns are unique to an individual and of possible value for identity testing.

Single strand conformational polymorphism (SSCP) yields similar results. In this method the PCR amplified DNA is denatured and sequence dependent conformations of the single strands are detected by their differing rates of migration during gel electrophoresis. As with LSSP-PCR above, different patterns are obtained that signal differences in sequence. However, neither LSSP-PCR nor SSCP gives specific sequence information and both depend on the questionable assumption that any base that is changed in a sequence will give rise to a detectable conformational change. Pastinen (Clin. Chem. (1996) 42:1391) amplifies the target DNA and immobilizes the amplicons. Multiple primers are then allowed to hybridize to sites 3′ and contiguous to a SNP (single nucleotide polymorphism) site of interest. Each primer has a different size that serves as a code. The hybridized primers are extended by one base using a fluorescently labeled dideoxynucleoside triphosphate. The size of each of the fluorescent products that is produced, determined by gel electrophoresis, indicates the sequence and, thus, the location of the SNP. The identity of the base at the SNP site is defined by the triphosphate that is used. A similar approach is taken by Haff (Nucleic Acids Res. (1997) 25:3749) except that the sizing is carried out by mass spectrometry and thus avoids the need for a label. However, both methods have the serious limitation that screening for a large number of sites will require large, very pure primers that can have troublesome secondary structures and be very expensive to synthesize.

Hacia (Nat. Genet. (1996) 14:441) used a high-density array of oligonucleotides. Labeled DNA samples were allowed to bind to 96,600 20-base oligonucleotides, and the binding patterns produced from different individuals were compared. The method is attractive in that SNPs can be directly identified, but the cost of the arrays is high, and non-specific hybridization may confound the accuracy of the genetic information.

Fan (Oct. 6-8, 1997 IBC, Annapolis Md.) has reported results of a large scale screening of human sequence-tagged sites. The accuracy of single nucleotide polymorphism screening was determined by conventional ABI resequencing.

Ross, in Anal. Chem. (1997) 69:4197, discusses allele specific oligonucleotide hybridization along with mass spectrometry.

Brenner and Lerner, in PNAS (1992) 89:5381, teach that compounds prepared by combinatorial synthesis can each be labeled with a characteristic DNA sequence. If a given compound proves of interest, the corresponding DNA label is amplified by PCR and sequenced, thereby identifying the compound.

W. Clark Still, in U.S. Pat. No. 5,565,324 and in Accounts of Chem. Res. (1996) 29:155, uses a releasable mixture of halocarbons on beads to code for a specific compound on the bead that is produced during synthesis of a combinatorial library. Beads bearing a compound of interest are treated to release the coding molecules, and the mixture is analyzed by gas chromatography with flame ionization detection.

U.S. Pat. No. 5,807,682 describes probe compositions for detecting a plurality of nucleic acid targets.

### SUMMARY OF THE INVENTION

In one aspect, the invention provides a method for isolating a signal produced by a detectable moiety of a released predetermined portion of a probe from signal obtained from intact probe and from other fragments thereof. The method comprises:
(a) combining a probe which comprises a first predetermined portion which is releasable and includes a detectable moiety, and a second portion which includes at least one interactive functionality, with a reagent that potentiates the release of the first portion from the probe;
(b) subjecting the resulting mixture to conditions under which the probe is cleaved, such that the first portion is released from the probe;
(c) contacting the mixture with a capture reagent which binds the interactive functionality, and
(d) separating the capture agent or the released predetermined first portion from the mixture.

In one embodiment, the interactive functionality is a ligand, which may be, for example, a small molecule, a receptor for a small molecule, an antigen, an antibody, or an oligonucleotide. In another embodiment, the interactive functionality is a chelating moiety, and the capture agent comprises boron, arsenic, a transition metal, or a ferrocene; or vice versa.

When the probe comprises an oligonucleotide, the releasable first portion is preferably attached at a nucleotide thereof, and an organic moiety comprising the interactive functionality is attached at a nucleotide adjacent to the nucleotide to which the releasable first portion is attached. For example, the releasable first portion may be attached is a terminal nucleotide, and the interactive functionality at the adjacent, penultimate nucleotide.

The releasable first portion preferably has a predetermined mass or mobility. In a preferred embodiment, the probe comprises a structure (D, Mⱼ)-L-, where
(i) D is a detection group comprising a detectable label;
(ii) L is a cleavable linkage, and
(iii) Mⱼ is a mobility modifier having a mass or charge/mass ratio that imparts a unique and known electrophoretic mobility to a corresponding reporter of the form (D,Mⱼ)-L′, within a selected range of mobilities, with respect to other reporters of the same form in a set of such probes, where L′ is the residue of L following cleavage.

In a related aspect, the invention provides a related method for detecting binding of or interaction between a target binding agent and any of a plurality of probes, comprising:
(a) subjecting a mixture comprising the target binding agent and the probes to conditions for interaction therebetween,
wherein each probe comprises (i) a cleavable reporter group, comprising a detectable moiety and having a mobility, when cleaved, that is unique to the probe among the plurality of probes, and (ii) at least one interactive functionality adjacent the cleavable reporter group;
(b) subjecting the mixture to conditions under which the cleavable moiety is cleaved to release the reporter groups, wherein said cleavage is dependent on said interaction between the target binding agent and any of the plurality of probes;
and wherein the interactive functionality enables the isolation of signal produced by the detectable moieties of the released reporter groups from signal obtained from intact probes and from other fragments thereof,
(c) separating the released reporter groups by their differences in mobility; and
(d) detecting the detectable moieties of released reporter groups, to determine binding of or interaction between the target binding agent and each probe.

In one embodiment of this method, the interactive functionality is effective to quench signal produced by the detectable moiety in intact probe, but not in released reporter group. In other embodiments, the isolation is effected by contacting the mixture of (b) with a capture reagent which binds the interactive functionality, and separating the capture agent or the released reporter groups from the mixture. Examples of interactive functionalities and capture agents are as described above.

Again, in a preferred embodiment, each probe comprises an oligonucleotide to which the cleavable reporter group is attached at a nucleotide thereof, and an organic moiety comprising the interactive functionality is attached at a nucleotide adjacent to the nucleotide to which the reporter group is attached. The reporter group may be attached to a terminal nucleotide, as described above.

In another aspect, the invention provides kits for use in detecting the presence and/or amount of each of a plurality of target molecules. Such a kit includes, in packaged combination, a plurality of electrophoretic probes, each capable of target-specific binding to a binding site on a selected target molecule, and each having (i) a unique cleavable reporter group, having a detectable reporter moiety and a unique electrophoretic mobility which allows the reporter group to be uniquely identified among all other reporter groups associated with the plurality of probes, and (ii) at least one interactive functionality adjacent the reporter group, wherein the interactive functionality is effective in isolating the signal produced by released reporter groups from the signal produced by intact probes and other fragments thereof. Preferably, the kit also includes one or more capture agents effective to bind to the interactive functionality.

In selected embodiments, the interactive functionality on each probe is a chelating moiety, and the capture reagent comprises boron, arsenic, a transition metal, or a ferrocene; or vice versa Each probe may comprises an oligonucleotide, to which the cleavable reporter group is attached at a nucleotide thereof, e.g. a terminal nucleotide, and an organic moiety comprising an interactive functionality attached at a nucleotide adjacent to the nucleotide to which the cleavable reporter group is attached, e.g. the penultimate nucleotide.

In a further aspect, the invention provides a synthetic biopolymer, such as a polynucleotide or polypeptide, or a nucleoside, incorporating an interactive functionality. Such a synthetic biopolymer or nucleoside has conjugated thereto a moiety having the formula Ya: 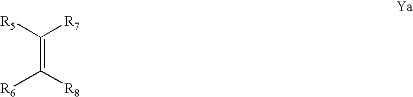
where R⁵ and R⁶ are independently selected from hydroxy, amino, substituted amino, carbonyl, carboxamide and N-hydroxy amide, any of which may be bound to a protecting group, and R⁷ and R⁸ are independently carboxy or cyano or together form one or more rings, wherein each ring comprises 5 to 10 atoms and one or more double bonds, in addition to the double bond of the above formula, and wherein the atoms of each ring are optionally substituted with alkyl, hydroxy, nitro, or oxo.

In selected embodiments, R⁷ and R⁸ together form a benzene or anthracene ring. One embodiment of Ya has the formula Yb: 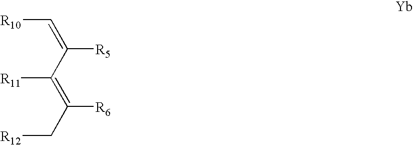
where R⁵ and R⁶ are independently selected from hydroxy, amino, substituted amino, carbonyl, carboxamide or N-hydroxy amide, any of which may be bound to a protecting group, and R¹⁰ and R¹¹ and R¹² are independently carboxyl or cyano, or together form one or more rings, wherein each ring comprises 5 to 10 atoms and one or more double bonds, in addition to the double bonds of the above formula, and wherein the atoms of each ring are optionally substituted with alkyl, hydroxy, nitro or oxo. In one embodiment, R¹⁰ and R¹¹ and R¹² together form a naphthalene ring.

Another embodiment of Ya has the formula Ya: 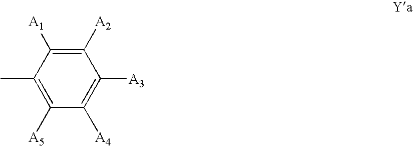
where A¹ to A⁵ are independently selected from hydrogen, hydroxy, amino, substituted amino, carbonyl, carboxamide and N-hydroxy amide, any of which may be bound to a protecting group, wherein at least two adjacent members of A¹ to A⁵ are not hydrogen.

Still another embodiment of Ya, and an embodiment of Yb, has the formula Y′b: 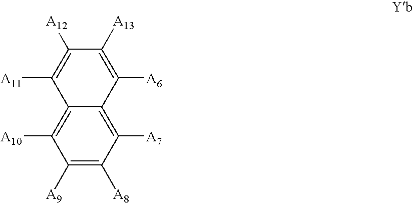
where A⁶ and A⁷ are independently selected from hydroxy, amino, substituted amino, carbonyl, carboxamide and N-hydroxy amide, any of which may be bound to a protecting group, one of A⁸ to A¹³ is linked to said synthetic biopolymer or biopolymer precursor by a bond or a linking group, and the remaining members of A⁸ to A¹³ are independently hydrogen, hydroxy, amino, substituted amino, carbonyl, carboxamide or N-hydroxy amide.

In selected embodiments, the moiety comprises a 1,2-diol, 1,3-diol, 1,2-aminoalcohol, 1,3-aminoalcohol, 1,2-hydroxy acid, 1,3-hydroxy acid, 1,2-hydroxy acid amide, 1,3-hydroxy acid amide or dioxime. In more specific embodiments, the moiety is selected from the group consisting of ortho-hydroxybenzenes (catechols), ortho-hydroxybenzohydroxamic acids, ortho-hydroxybenzoic acids, ortho-hydroxybenzamides, imidazolyl phenols, dihydroxyfumaric acids, hydroxy pyridine aldehydes and hydroxyanthraquinone dioximes.

In still further embodiments, the moiety is selected from the group consisting of catechol, salicylamide, N-phenylsalicylamidine, 2-(4′imidazolyl)phenol, 1,8-dihydroxynaphthalene, dihydroxyfumaric acid, salicylaldehyde, 3-hydroxypyridine-4-aldehyde, pyridoxamine, 1-hydroxyanthraquinone dioxime and disalicylimide.

A nucleoside compound having an interactive functionality includes compounds of the formula: 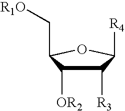
wherein:
R¹ is H, dimethoxytrityl DMT), triphosphate ester, diphosphate ester, or monophosphate ester;
R² is H or phosphoramidite;
R³ is H, OH, ODMT, or OX(Y)ₚ, wherein X is a bond or a linking group;
and R⁴ is one of: 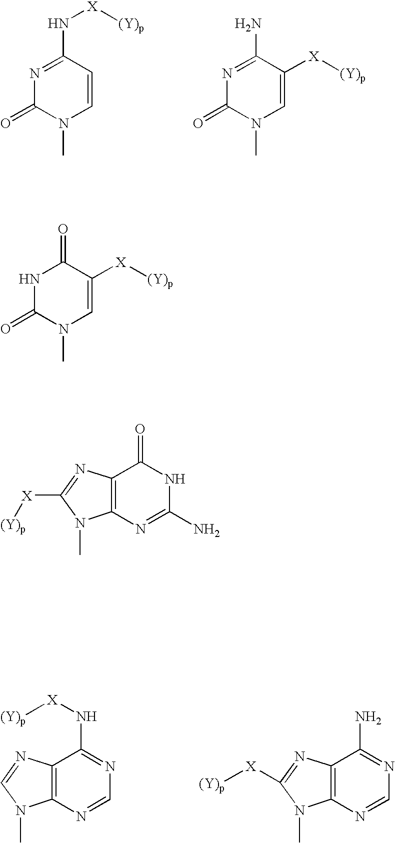
   wherein:
Y is Ya or Yb: 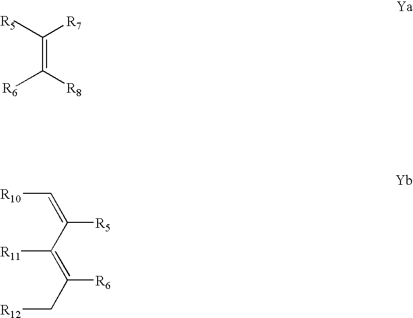
   where
R⁵ and R⁶ are independently selected from hydroxy, amino, substituted amino, carbonyl, carboxamide or N-hydroxy amide, any of which may be bound to a protecting group, R⁷ and R⁸ are independently carboxy or cyano or together form one or more rings, wherein each ring comprises 5 to 10 atoms and one or more double bonds, in addition to the double bond of the formula Ya, wherein the atoms of each ring are optionally substituted with alkyl, hydroxy, nitro, or oxo;
R¹⁰ and R¹¹ and R¹² are independently carboxyl or cyano or together form one or more rings, wherein each ring comprises 5 to 10 atoms and one or more double bonds, in addition to the double bonds of the formula Yb, wherein the atoms of each ring are optionally substituted with alkyl, hydroxy, nitro or oxo;
X is linked to Ya through R⁷ or R⁸ or to Yb through R¹⁰ or R¹¹ or R¹²; each p in R³ and R⁴ is independently 0 to 3, and p in at least one of R³ or R⁴ is 1.

Various embodiments of the groups Ya and Yb are as described above. In preferred embodiments of Ya and Yb, R⁵ and R⁶ are hydroxy.

In selected embodiments, Ya is selected from the group consisting of ortho-hydroxybenzenes (catechols), ortho-hydroxybenzohydroxamic acids, ortho-hydroxybenzoic acids, ortho-hydroxybenzamides, imidazolyl phenols, dihydroxyfumaric acids, and hydroxy pyridine aldehydes, or Yb is a hydroxyanthraquinone dioxime. In more specific embodiments, Ya is selected from the group consisting of catechol, salicylamide, N-phenylsalicylamidine, 2-(4′-imidazolyl)phenol, 1,8-dihydroxynaphthalene, dihydroxyfumaric acid, salicylaldehyde, 3-hydroxypyridine-4-aldehyde, pyridoxamine and disalicylimide, or Yb is 1-hydroxyanthraquinone dioxime.

Also provided are oligonucleotides or polynucleotides having conjugated thereto a nucleoside compound having an interactive functionality, as shown above.

In a related aspect, the invention provides a method of synthesizing an oligonucleotide of predetermined length, said method comprising reacting activated nucleoside monomer reagents sequentially until said oligonucleotide of predetermined length is formed, wherein at least one of said nucleoside monomer reagents is a nucleoside compound having an interactive functionality, as shown above. The method preferably includes comprising purifying the oligonucleotide by contacting a reaction mixture containing the oligonucleotide with a boronate, to form a boronate complex therewith, and separating the complex from said mixture. Preferably, the boronate is attached to a solid support to facilitate the separation.

### BRIEF DESCRIPTION OF THE DRAWINGS

FIGS. 1A, B and C depict the SNP detection sequences for two SNP alleles (A), the optical characteristics of the fluorescent dyes (B), and the cleaved fragments from the SNP detection sequences (C).

FIG. 2 shows the structure of several benzoic acid derivatives that can serve as mobility modifiers.

FIGS. 3A-C provide a schematic illustration of the advantages of the invention, in one embodiment; where (A) shows a 5′ exonuclease cleavase assay in the absence of any interactive or capture reagent; (B) shows the use of biotin attached at the 3′ terminus of the probe, to facilitate the removal of uncleaved or partially cleaved e-tag probe from the reaction mixture; and (C) shows the use of biotin attached adjacent to the cleavable reporter group, as in an embodiment of the present invention, and the resulting electropherograms in each case. FIG. 3D shows an example of a nuclease resistant modification (phosphorothioate) to the backbone of a target binding region.

FIG. 4 illustrates one design of e-tag reagents using standard phosphoramidite coupling chemistry.

FIG. 5 illustrates e-tag reporter groups and their electrophoretic separation (elution) times on a LabCard™ (ACLARA Biosciences Inc.). (Detection: 4.7 cm; 200 V/cm.)

FIG. 6 provides predicted and experimental (*) elution times of e-tag reporters separated by capillary electrophoresis. C₃, C₆, C₉, and C₁₈ are commercially available phosphoramidite spacers from Glen Research, Sterling Va. C₃ is DMT (dimethoxytrityl)oxypropyl Q; C₆ is DMToxyhexyl Q; C₉ is DMToxy(triethyleneoxy) Q; C₁₂ is DMToxydodecyl Q; and C₁₈ is DMToxy(hexaethyleneoxy)Q, where Q is N,N-diisopropyl-O-cyanoethyl phosphoramidite.

FIG. 7 illustrates several possible structural variations for preparation of several mobility-modified nucleic acid phosphoramidites, useful for the penultimate coupling during e-tag probe synthesis on a standard DNA synthesizer.

FIG. 8 shows multiple electropherograms showing separation of individual e-tag reporters. The figure illustrates obtainable resolution of the reporters, which are identified by ACLA numbers (see FIG. 17).

FIG. 9 shows charge modified phosphoramidites, where EC and CE represent cyanoethyl.

FIG. 10 shows polyhydroxylated charge modifier phosphoramidites.

FIG. 11 illustrates one exemplary synthetic approach to preparation of etag reporter groups, starting with commercially available 6-carboxy fluorescein.

FIG. 12 illustrates generation of diversity in etag reporters via the use of a symmetrical bis-amino alcohol linker coupled with a multitude of carboxylic acid derivatives.

FIG. 13 illustrates an alternative synthetic strategy starting with 5-aminofluorescein.

FIG. 14 illustrates commercially available diacid dichlorides and amino alcohols that can be used for synthesis of etag libraries.

FIG. 15 gives the structure of several e-tag moieties derived from maleimide-linked precursors.

FIG. 16 is a diagram of a system for performing multiplexed determinations using e-tag reagents.

FIGS. 17A-J shows the structures of numerous exemplary e-tag reporters.

FIGS. 18A and B depict the CE separation of the reaction products of Allele 1 after 0 and 40 cycles. Peaks: P=unreacted SNP detection sequence or e-tag probe, P′=SNP detection sequence or e-tag reporter product, TET=tetrachlorofluorescein Example 4).

FIGS. 19A and B depict the CE separation of the reaction products (or e-tag reporters) of Allele 2 after 0 and 40 cycles. FAM=fluorescein (Example 4).

FIG. 20 is a graph of the CE separation of a 1:1 mixture of the 40 cycles products of Alleles 1 and 2, with experimental conditions as described for FIG. 18 (Example 4).

FIG. 21 is a graph of the CE separation of a 1:10 mixture of the 40 cycles products of Alleles 1 and 2, with experimental conditions as described for FIG. 18 (Example 5).

FIG. 22 is an electropherogram of electrophoretic tags for electrophoresis differing by a 1000-fold concentration.

FIGS. 23A-E and G are electropherograms from analysis of 5 SNPs of the cystic fibrosis genes, using multiplexed PCR and the subject e-tag probes. Three individual SNP loci and a triplex reaction are shown, using multiplexed PCR and the subject e-tag probes (FIGS. 23A-E and G), along with an image of agarose gel separation of the triplex reaction (23F) (Example 7).

FIG. 24 is an electropherogram of a separation of nine negatively charged e-tag reporters.

FIGS. 25A-D are electropherograms of probes employing a penultimate thiophosphate linkage in the e-tag probes to inhibit cleavage after the first phosphate linkage. FIGS. 25A and B reflect the results of experiments showing the formation of 5 different cleavage products in the PCR amplification of ANF (anti-nuclear factor) with (A) and without (B) the thiophosphate linkage. FIGS. 25C and D reflect the results of experiments showing the formation of 5 different cleavage products in the PCR amplification of GAPDH, with (C) and without (D) the thiophosphate linkage (Example 9).

FIG. 26 shows multiple electropherograms from a separation on a 310 analyzer, after an amplification reaction in the presence of probe and primer, and without the addition of avidin (Example 11).

FIG. 27 shows multiple electropherograms from a separation on a 310 analyzer, after an amplification reaction in the presence of probe and primer, and with the addition of avidin (Example 11).

FIGS. 28A-C are schematic illustrations of a CE² LabCard™ device (28A) and exemplary high voltage configurations utilized in this device for the injection (28B) and separation (28C) of products of an enzyme assay (Example 13).

FIG. 29 shows two electropherograms demonstrating e-tag reporter analysis using a CE² LabCard™. The figure shows the separation of purified labeled aminodextran with and without sensitizer beads. The addition of the sensitizer beads leads to the release of the e-tag reporter from the aminodextran, using singlet oxygen produced by sensitizer upon irradiation at 680 nm. Experimental conditions: Separation buffer 20 mM HEPES pH=7.4 with 0.5% PEO; voltage configurations as described for FIG. 28; assay mixture contained 29 μg/ml streptavidin coated sensitizer beads and was irradiated for 1 min at 680 nm using 680±10 nm filter and a 150 W lamp (Example 13).

FIG. 30 shows multiple electropherograms demonstrating e-tag reporter analysis using a CE² LabCard™. The figure shows the separation of purified labeled aminodextran using different concentrations of sensitizer beads. The higher concentration of sensitizer beads leads to the higher release of e-tag reporters from the labeled aminodextran. Experimental conditions were as for FIG. 29 (Example 13).

FIG. 31 depicts the linear calibration curve for the release of e-tag reporters as a function of the sensitizer bead concentration. Results were obtained using a CE² LabCard™. Experimental conditions were as for FIG. 29 (Example 13).

FIG. 32 shows a data curve of the effect of the concentration of labeled aminodextran on the e-tag reporter release. As demonstrated in this figure, the lower concentration of labeled aminodextran for a given concentration of sensitizer beads leads to more efficient e-tag reporter release Results were obtained using a CE² LabCard™. Experimental conditions were as for FIG. 29 (Example 13).

FIG. 33 is a schematic diagram of the synthesis of the phosphoramidite of biotin-deoxycytosine (dC) (Reagent C).

FIG. 34 is a schematic diagram of the synthesis of the phosphoramidite of biotin-deoxyadenosine (dA) (Reagent D).

FIG. 35 is a formula depicting an embodiment of a 5′-catechol-DMT-phosphoramidite in accordance with the present invention.

FIG. 36 is a schematic depiction of typical results of a 5′-nuclease assay and a corresponding electropherogram without use of the present invention.

FIGS. 37-42 are schematic depictions of selected embodiments of the present invention, employing a modified residue at the penultimate position of an oligonucleotide electrophoretic probe, and corresponding electropherograms.

FIG. 43 is a schematic depiction of another embodiment of the present invention represented in Example 3 employing a modified residue, namely, a catechol, at the penultimate position of an oligonucleotide electrophoretic probe, and a corresponding electropherogram, after treatment of the reaction mixture with a boronic acid matrix.

FIGS. 44-48 depict electropherograms demonstrating the results of Example 3 in removing unwanted materials from materials of interest.

FIG. 49 is a formula depicting an embodiment of a 3′-CPG-catechol-DMT in accordance with the present invention.

FIGS. 50A-E depict embodiments of a dCTP-catechol, a dTTP-catechol, a dATP-catechol, a dGTP-catechol, and a dUTP-catechol, respectively, in accordance with the present invention.

FIGS. 51A-E depict embodiments of a dC-catechol phosphoramidite, a dT-catechol phosphoramidite, a dA-catechol phosphoramidite, a dG-catechol phosphoramidite, and a dU-catechol phosphoramidite, respectively, in accordance with the present invention.

FIGS. 52-53 depict various catechol compounds modified with linking functionalities for incorporation of the catechol moiety into a reagent.

FIG. 54 depicts formulas for various phenylboronic acids and their respective pKa's.

FIG. 55 depicts formulas for various boronate affinity matrices.

FIG. 56 is a reaction scheme for the preparation of a fully protected catechol-NHS compound.

FIG. 57 is a reaction scheme for the synthesis of an oligomer containing a catechol moiety.

FIGS. 58-62 show reaction schemes for the synthesis of phosphoramidites of catechol-dA, catechol-dG, catechol-dC, catechol-dT, and biotin-dG, respectively.

### DESCRIPTION OF THE SPECIFIC EMBODIMENTS

I. Definitions

The discussion in this application may be viewed in reference to U.S. patent application Ser. No. 09/698,846, filed 27 Oct., 2000, which is a CIP of Ser. No. 09/602,586, filed 21 Jun., 2000, which, with Ser. No. 09/684,386, filed 4 Oct., 2000 are CIP's of Ser. No. 09/561,579, filed 28 Apr. 2000, which is a CIP of Ser. No. 09/303,029, filed 30 Apr., 1999.

An “electrophoretic probe” (which may also be referred to as an e-tag probe or electrophoretic tag probe) is a compound useful in the assays described herein, having four basic components or moieties: (i) a detection group or moiety D, (ii) a mobility or mass modifier M, (iii) a target-binding moiety T, and (iv) a linking group L that links the mobility modifier and detection group to the target-bonding moiety. These terms will first be examined in the context of the functioning of the electrophoretic probes in the invention, then more fully defined by their structural features.

The function of an electrophoretic probe in the methods described herein is to interact with a target, such as a single-stranded nucleic acid, a protein, a ligand-binding agent, such as an antibody or receptor, or an enzyme, e.g., as an enzyme substrate. The “portion”, “region” or “moiety” of the probe which binds to the target is the “target-binding moiety” or “target-binding region” or “target-binding portion” (“T”). After the target-binding moiety of an electrophoretic probe binds to a target, and typically as a result of such binding, the linking group of the electrophoretic probe may be cleaved to release an “electrophoretic tag” (or “e-tag” or “e-tag reporter”) that has, relative to other e-tags of the set employed in the assay, a unique mass or charge-to-mass ratio, rendering such e-tags separable by, for example, electroseparation or mass spectrometry. In one embodiment, the e-tags have a unique electrophoretic mobility in a defined electrophoretic system. The e-tag reporter is composed of the detection group (D), mobility modifier (M), and any residue of the linking group (L′) that remains associated with released reporter e-tag after cleavage. Therefore, a further function of an electrophoretic probe is to release an e-tag reporter, which can be identified according to its distinct and known electrophoretic mobility.

A set of electrophoretic probes of the invention can be represented by the following formula:
(D, Mⱼ)-L(-Z)ᵥ-Tⱼ,
where D is a detection moiety, Mⱼ is the jth mobility modifier, Z is an organic moiety comprising interactive functionalities, where Z is adjacent the cleavable linkage (e.g., when the linking group is the 5′-terminal nucleotide of an oligonucleotide target binding moiety, Z is on the penultimate nucleotide), v is 1 to about 10, preferably 2 to about 8, more preferably 3 to about 6, Tj is the jth target binding agent, and the linking group is represented by L. In this structural designation, (D,Mⱼ)- indicates that either the detection group or the mobility modifier is joined to the linking group.

According to an important feature of the invention, there is provided a set of electrophoretic probes, each of which has a unique target-binding moiety, with respect to species being assayed, and an associated “e-tag moiety” that imparts to the associated e-tag reporter, a unique electrophoretic mobility with respect to any other e-tag moiety of the set, by virtue of a unique charge to mass ratio. A “set” or “group”, “plurality” or “library” of electrophoretic probes refers to a plurality of electrophoretic probes having typically at least five, typically 10-100 or more probes with different unique target-binding moieties and associated e-tag moieties. Such a set can be used for detecting each or any of a plurality of known, selected target nucleotide sequences, or for detecting the binding of, or interaction between, each or any of a plurality of ligands and one or more target antiligands.

The term “target-binding moiety” or “Tⱼ” refers to the component of an e-tag probe that participates in recognition and specific binding to a designated target. The target-binding moiety may also be referred to as T or T′, or may be defined based on the type of target, e.g., as a SNP detection sequence or an oligonucleotide detection sequence. Each Tⱼ, j=1 to j=n is different, i.e., will bind specifically and/or with distinct affinities to different targets.

As used herein, the term “e-tag” or “e-tag reporter” refers to the cleavage product generated as a result of the interaction between an electrophoretic probe and its target. An e-tag reporter resulting from the interaction of an e-tag probe used to detect the binding of or interaction between a ligand and an antiligand typically has the form (D,Mⱼ)-L′. D and Mⱼ are defined above and L′ is the residue of L that remains attached to (D, Mⱼ) after an e-tag reporter is cleaved from the corresponding e-tag probe. The cleavage product may also include a residual portion of the target binding moiety (T), such as one or more nucleotides when the target-binding moiety comprises an oligonucleotide. E-tag reporters can be differentiated by electrophoretic mobility or mass and are amenable to electrophoretic separation and detection, although other methods of differentiating the tags may also find use.

The “detection group” (D) is a chemical group or moiety that is capable of being detected by a suitable detection system, particular in the context of detecting molecules containing the detection group after or during electrophoretic separation. The detection group in accordance with the present invention is preferably a fluorescent compound as disclosed herein. The fluorescent compounds can be readily detected during or after electrophoretic separation of molecules by illuminating the molecules with a light source in the excitation wavelength and detecting fluorescence emission from the irradiated molecules. The detection group is typically common among a set or subset of different electrophoretic probes, but may also differ among probe subsets, contributing to the unique electrophoretic mobilities of the released e-tag reporter.

The “mobility modifier” (M) is a generally a chemical group or moiety that is designed to have a particular charge to mass ratio, and thus a particular electrophoretic mobility in a defined electrophoretic system. Exemplary types of mobility modifiers are discussed below. In a set of n electrophoretic probes, each mobility modifier is designated Mⱼ, where j=1 to n, as above. The mobility modifier may be considered to include a mass-modifying region and/or a charge-modifying region or a single region that acts as both a mass- and charge-modifying region. The mobility modifying region may also be referred to as a mobility/mass identifying region or “MIR”, a charge-imparting moiety, and/or a mobility region.

The linking group (L) may be a covalent bond that is cleavable under selected cleaving conditions, or a chemical moiety or chain, such as, for example, a nucleotide and associated phosphodiester bond, an oligonucleotide with an internal cleavable bond, an oligopeptide, or an enzyme substrate that contains a cleavable bond. Cleavage typically occurs as the result of binding of the probe to the target, which is followed by enzymatic or otherwise facilitated cleavage of the linking-group bond.

The linking group may or may not contribute a linking-group “residue” (L′) to the released e-tag reporter, also dependent on the nature of the lining group and the site of cleavage. For example, where the linking group is a covalent bond, or cleavage of the linking group occurs immediately adjacent the “e-tag moiety”, the linking group leaves no residue, i.e., it does not contribute additional mass or charge to the released e-tag reporter. Similarly, where the linking group is a chemical group or chain that is cleaved internally or immediately adjacent the target-binding moiety, cleavage of the liming group leaves a residual mass and possible charge contribution to the released e-tag reporter. In general, this contribution is relatively small, and is the same for each different released e-tag, assuming a common linking group within the probe set. As such, generally, the residue will not significantly affect the relative electrophoretic mobilities of the released e-tag reporters, nor the ability to resolve the e-tag reporters into electrophoretic species that can be uniquely identified.

In general, the unique charge to mass ratio of an e-tag moiety is due to the chemical structure of the mobility modifier, since the detection group and linking-group residue (if any) are generally common to any set of electrophoretic probes. However, it is recognized that the detection group can make unique charge and/or mass contributions to the e-tag reporters as well. For example, a set of electrophoretic probes may be made up of a first subset having a group of mobility modifiers that impart unique electrophoretic mobilities to the subset in combination with a detection group having one defined charge and/or mass, and a second subset having the same group of mobility modifiers in combination with a second detection group with a different charge and/or mass, thus to impart electrophoretic mobilities which are unique among both subsets.

Electrophoretic probes of the invention find utility in performing multiplexed for detection/analysis of targets including, but not limited to nucleic acid detection, such as sequence recognition, SNP detection, transcription analysis or mRNA determination, allelic determination, mutation determination, BLA typing or MHC determination and haplotype determination, in addition to detection of other ligands, such as proteins, polysaccharides, etc. In selected embodiments, the target binding moiety of an electrophoretic probe is a SNP detection sequence, a fluorescence SNP detection sequence or an oligonucleotide detection sequence.

As used herein, the term “binding event” generally refers to the binding of the target-binding moiety of an e-tag probe to its target. By way of example, such binding may involve the interaction between complementary nucleotide sequences or the binding between a ligand and target antiligand.

As used herein, the term “organic moiety comprising an interactive functionality” generally refers to at least one moiety positioned in the electrophoretic probe such that the interactive functionality does not form part of the released detection group and mobility modifier (i.e. the released etag reporter group) upon release thereof from the electrophoretic probe. The organic moiety comprising an interactive functionality enables signal from the detection group to be isolated from signal produced by other components of the aforementioned system. The interactive functionality may be a group included within the target binding portion of an e-tag probe that is capable of binding specifically to a “capture agent”. This interaction may be used to separate uncleaved e-tag probes and/or unwanted cleavage products from released e-tag reporters.

II. Compositions for Multiplexed Assays

The subject invention provides compositions and methods for improved analysis of complex mixtures, which may be employed,; for example, in the simultaneous identification of a plurality of entities, such as nucleic acid or amino acid sequences, SNP's, alleles, mutations, proteins, haptens, protein family members, expression products, etc. To this end, libraries of differentiable compounds or “e-tags” are provided, where each compound comprises a mass-identifying region or mobility modifier (M) that provides for ready identification by electrophoresis or mass spectrometry, by itself or in conjunction with a detectable label (D).

Depending on the assay, the e-tag product generated in the assay may also include e.g. one or more nucleotides or amino acids, resulting from the release of e-tag from a target-binding region of an electrophoretic probe, as described herein. The e-tag reporters are generated as the result of the interaction between an electrophoretic probe, which comprises an e-tag joined to a target-binding moiety, and a corresponding target.

In addition, the subject invention employs a variety of reagent systems. The reagent system, in response to a binding event between a target-binding moiety and a target, effects a change in the electrophoretic probe, to generate a product having a mobility and/or mass different from the starting material. The reagent system frequently comprises an enzyme and may comprise the target to which the probe binds. The effect of the reagent system is to make or break a bond by physical, chemical or enzymatic means. Typically, the reagent system is effective to cleave the electrophoretic probe and release the e-tag reporter. Each of the products of the different e-tag containing target-binding moieties can be accurately detected, so as to determine the occurrence of the binding event.

The compositions may be used for a variety of multiplexed analyses involving a plurality of probes. The subject systems find use, for example, in nucleic acid and protein analyses, reactions, particularly enzyme reactions, where one or more enzymes are acting on a group of different potential or actual substrates.

Accordingly, a system is provided for the simultaneous multiplexed determination of a plurality of events, employing electrophoresis to distinguish the events, comprising: (i) an electrophoretic device for electrophoretic separation and detection, (ii) a set of electrophoretic probes, and (iii) a reagent composition comprising at least one active agent effective to modify at least one probe, to produce an e-tag reporter, and (iv) means for transferring the produce reporter(s) to the electrophoretic device for separation and detection. The electrophoretic device may be connected to a data processor for receiving and processing data from the device, as well as operating the electrophoretic device.

As described above, the electrophoretic probes comprise, in general, a mobility modifying region (M) and detection group (D), which together form the e-tag reporter, joined to a target binding moiety (T) by a linker (L). The target binding moiety preferably has a high specific binding affinity for a reciprocal binding member, the analyte. Usually, the binding affinity will be at least about 10⁻⁷M⁻¹, more usually, at least about 10⁻⁸M⁻¹.

The system include libraries of probes, where a library or probes comprises a plurality of e-tags having a corresponding plurality of different mobility-modifying regions, so as to be separable by electrophoresis. Upon binding to a target in an assay, the probe is modified by the gain and/or loss of a group that changes the mass and may also change the charge. Typically, the e-tag is joined to the target-binding moiety by a cleavable bond, and the e-tag is released for analysis subsequent to the binding event, e.g. complex formation.

The libraries will ordinarily have at least about 5 members, usually at least about 10 members, and may have 100 members or more, for convenience generally having about 50-75 members. Diversity can be achieved by variation of the linker and/or mobility modifier, the chemical and optical characteristics of the fluorescer, the use of energy transfer complexes, and the like.

Depending upon the probe to which the e-tag moiety is attached, there may be a single e-tag group or a plurality of e-tag groups, generally ranging from about 1 to about 100, more usually ranging from about 1 to about 40, more particularly ranging from about 1 to about 20. The number of e-tag groups bonded to a single target-binding moiety will depend upon the sensitivity required, the solubility of the e-tag conjugate, the effect on the assay of a plurality of e-tag probes, and the like.

A. Electrophoretic Tags (eTags)

The electrophoretic tag is a water soluble compound that is stable with respect to the conditions employed for cleavage and release and that includes a detectable group. Otherwise, the tag may vary widely in size and structure. Preferably, the tag carries a charge at neutral pH and has a molecular weight in the range of from about 150 to about 10,000 daltons, more preferably, from about 150 to about 5000 daltons, and most preferably, from about 150 to 2500 daltons. Preferred structures are described more fully below. Preferably, the detection group generates an electrochemical, fluorescent, or chromogenic signal. Most preferably, the detection group generates a fluorescent signal.

A description of etags is also provided in co-owned U.S. application Ser. No. 09/824,851, published on Dec. 13, 2001 as U.S. application No. 20010051340. The descriptions therein are primarily directed to phosphoramidate-based eTags, but can be adapted to other structures.

Each of the plurality of electrophoretic tags employed in a single multiplexed assay has a distinct charge-to-mass ratio and/or a unique optical property with respect to the other members of the plurality. Preferably, the optical property is a fluorescence property, such as emission spectrum, fluorescence lifetime, or the like. More preferably, the fluorescence property is an emission spectrum. For example, each electrophoretic tag of a plurality of tags may have the same fluorescent emission properties, but will differ from the others by virtue of unique charge-to-mass ratios. On the other hand, two or more of the electrophoretic tags of a plurality of tags may have identical charge-to-mass ratios, but they will have unique fluorescent properties, e.g. spectrally resolvable emission spectra, so that all the members of the plurality are distinguishable by the combination of electrophoretic separation and fluorescence measurement.

A preferred structure of an electrophoretic tag can be represented by (M,D), where M is a mobility-modifying moiety and D is a detection moiety. The notation “(M,D)” is used to indicate that the ordering of the M and D moieties may be such that either moiety can be adjacent to the point of linkage to the probe.

A1. Detection Moiety D

Detection moiety D is any moiety that allows the product and substrate to be detected, preferably quantitatively, following separation in the separation medium. For spectrophotometric detection, fluorophores or dyes may be used. Radiolabeled reporters are another class of suitable reporters. Alternatively, the reporter may be a catalytic moiety that is effective to catalyze a detectable reaction in the presence of suitable reaction components, such as described in co-owned U.S. patent application Ser. No. 60/293,821, filed May 26, 2001 and PCT Appn. No. WO 2002US16726, filed May 24, 2002.

Preferably, D is a fluorescent dye. Exemplary fluorescent dyes for use with the invention include water-soluble rhodamine dyes, fluoresceins, 4,7-dichlorofluoresceins, benzoxanthene dyes, and energy transfer dyes, disclosed in the following references: Handbook of Molecular Probes and Research Reagents, 8th ed., (Molecular Probes, Eugene, 2002); Lee et al., U.S. Pat. No. 6,191,278; Lee et al., U.S. Pat. No. 6,372,907; Menchen et al., U.S. Pat. No. 6,096,723; Lee et al., U.S. Pat. No. 5,945,526; Lee et al., Nucleic Acids Research, 25: 2816-2822 (1997); Hobb, Jr., U.S. Pat. No. 4,997,928; Khanna et al., U.S. Pat. No. 4,318,846; Reynolds, U.S. Pat. No. 3,932,415; Eckert et al., U.S. Pat. No. 2,153,059; Eckert et al., U.S. Pat. No. 2,242,572; Taing et al., PCT Pubn. No. WO 02/30944; Stryer, Science 162, 526 (1968) and Brand et al, Ann. Rev. Biochem. 41, 843 (1972). Further specific exemplary fluorescent dyes include 5- and 6-carboxyrhodamine 6G; 5- and 6-carboxy-X-rhodamine, 5- and 6-carboxytetramethylrhodamine, 5- and 6-carboxyfluorescein, 5- and 6-carboxy-4,7-dichlorofluorescein, 2′,7′-dimethoxy-5- and 6-carboxy-4,7-dichlorofluorescein, 2′,7′-dimethoxy-4′,5′-dichloro-5- and 6-carboxyfluorescein, 2′,7′-dimethoxy-4′,5′-dichloro-5- and 6-carboxy-4,7-dichlorofluorescein, 1′,2′,7′,8′-dibenzo-5- and 6-carboxy-4,7-dichlorofluorescein, 1′,2′,7′,8′-dibenzo-4′,5′-dichloro-5- and 6-carboxy-4,7-dichlorofluorescein, 2′,7′-dichloro-5- and 6-carboxy-4,7-dichlorofluorescein, and 2′,4′,5′,7′-tetrachloro-5- and 6-carboxy-4,7-dichlorofluorescein. Most preferably, D is a fluorescein or a fluorescein derivative. Many of these compounds are commercially available with substituents on the phenyl group which can be used as the site for bonding or as the bonding functionality. For example, amino and isothiocyanate substituted fluorescein compounds are available.

Other fluorescers include nitrogen-containing macrocycles, e.g. derivatives of porphyrins, azaporphyrins, cornins, sapphyrins and porphycenes and other like macrocycles, which contain electrons that are extensively delocalized. The azaporphyrin derivatives include phthalocyanine, benzotriazaporphyrin and naphthalocyanine and their derivatives. In some instances, fluorescent fusion proteins may be employed, using green fluorescent protein or other fluorescent protein fused to a polypeptide substrate.

The detection moiety of (M,D) may generate a fluorescenit signal by an energy transfer mechanism. Preferably, in this aspect, D has the form “D₁-g-D₂” where D₁ and D₂ are acceptor-donor pairs of molecules, e.g. Wu et al., Anal. Biochem. 218:1-13 (1994), and g is a rigid linker that maintains D₁ and D₂ at a substantially constant distance. Guidance in selecting the rigid linker may be found in Wu et al. (cited above) and in U.S. Pat. Nos. 5,863,727; 5,800,996; 5,945,526; and 6,008,379. Either D₁ or D₂ may be the acceptor and the other the donor molecule in the pair. Exemplary energy transfer detection moieties for use with the invention are disclosed in Lee et al., U.S. Pat. No. 5,945,526; Lee et al., Nucleic Acids Res. 25:2816-2822 (1997); Taing et al., PCT Publication WO 02/30944; and like references. Preferably, rigid: linker, g, is selected so that the distance between D₁ and D₂ is maintained at a substantially constant distance within the range of from 10-100 Angstroms. A wide variety of linking groups may be employed with the proviso that the linkage is stable to the presence of singlet oxygen. Preferably, D₁ and D₂ are selected from the set of fluorescein, rhodamine, rhodamine 6G, rhodamine 110, rhodamine X, tetramethylrhodamine, and halogenated derivatives thereof. More preferably, D₁ and D₂ are both fluorescein dyes.

In one aspect, g maybe selected from any of R₁—R₂—R₁ and R₁—R₂—C(═O)—X₁—R₃, the latter being present in either orientation with respect to D₁ and D₂; where X₁ is O, S, or NH; R₁ is (C₁-C₅ alkyldiyl, X₁, C(═O)) such that the moieties in parentheses are arranged in any linear order, R₂ is a 5 to 6 membered ring selected from the group consisting of cyclopentene, cyclohexene, cyclopentadiene, cyclohexadiene, furan, pyrrole, isopyrole, isoazole, pyrazole, isoimidazole, pyran, pyrone, benzene, pyridine, pyridazine, pyrimidine, pyrazine oxazine, indene, benzofuran, thionaphthene, indole and naphthalene; and R₃ is C₁-C₅ alkyldiyl.

An eTag may also contain, instead of a detectable group D, a functionality allowing it to bind to a detectable group D after reaction with a sample is complete. In one embodiment, a plurality of different functionalities are used for different binding members, for reaction with a label, and the different labels have corresponding functionalities that react with one of the first functionalities. For example, where the first functionalities include thiols, carboxyl groups, aldehydes and olefins, the labels could include activated olefins, alcohols, amines and thiol groups, respectively. By employing removable protective groups for one or more of the functionalities, the protective groups may be removed stepwise and the labels added stepwise, to avoid cross-reactivity.

A2. Mobility Modifier M

The mobility modifiers is selected to impart to the substrate and corresponding eTag a unique separation characteristic with respect to each other and all other substrates and corresponding eTags in the set. Where the separation characteristic is electrophoretic mobility, the mobility modifier will preferably be selected to impart a unique charge/mass ratio and/or shape to each substrate and product. Where the separation characteristic is chromatographic separation, the different mobility modifiers will have different hydrophobicities, charge, molecular weight, and/or size. For mass spectrometric analysis, the different mobility modifiers will have different masses.

Modifiers suitable for imparting different electrophoretic separation characteristics have been detailed in co-owned PCT patent application WO 00/66607, published Nov. 9, 2000, and incorporated herein by reference. Such modifiers typical have repeating subunit groups that impart unique charge/mass ratios to each different modifier.

Generally, each electrophoretic tag will have a charge/mass ratio in the range of about −0.0001 to 0.1, usually in the range of about −0.001 to about 0.5. Desirably, the difference in mobility among tags in a single assay, under the conditions of the determination, will be at least about 0.001, usually 0.002, more usually at least about 0.01, and may be 0.02 or more. Mobility is defined by q/M^{2/3}, where q is the charge on the molecule and M is the mass of the molecule.

In general, M is a chemical group or moiety having a particular charge-to-mass ratio and thus a particular electrophoretic mobility in a defined electrophoretic system. In a set of n electrophoretic probes, each unique mobility modifier may be designated Mⱼ, where j=1 to n, and n has a value as described above. That is, n is generally from 5 to 200, and more preferably, from 5 to 100, or 5 to 75, or from 5 to 50, or from 10 to 30.

The mobility-modifying moiety may be considered to include a mass-modifying region and/or a charge-modifying region, or a single region that acts as both a mass- and charge-modifying region. The mobility-modifying moiety may have one or more of the following characteristics: (i) a unique charge-to-mass ratio due to variations in mass, but not charge; (ii) a unique charge-to-mass ratio due to changes in both mass and charge; and (iii) a unique charge-to-mass ratio of between about −0.0001 and about 0.5, usually, about −0.001 and about 0.1. As noted above, D is typically the same among a set or plurality of different electrophoretic probes, but it may also differ among probe sets, contributing to the unique electrophoretic mobilities of the released electrophoretic tag.

The size and composition of mobility-modifying moiety, M, can vary from a bond to about 100 atoms in a chain, usually not more than about 60 atoms, and more usually not more than about 30 atoms, where the atoms are preferably selected from carbon, oxygen, nitrogen, phosphorous, boron and sulfur. Generally, when other than a bond, the mobility-modifying moiety has from about 0 to about 40, more usually from about 0 to about 30, heteroatoms, which in addition to the heteroatoms indicated above may include halogen or another heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (including ammonium), phosphonium, sulfonium, oxonium, etc., where substituents are generally aliphatic of from about 1-6 carbon atoms, the total number of carbon atoms per heteroatom usually being less than about 12, preferably less than about 9. Other substituents may include hydroxyl groups, including phenolic groups, carboxyl groups, esters, amides, phosphates, and heterocycles. The charged mobility-modifying moieties generally have only negative or only positive charges, although one may have a combination of charges, particularly where a region to which the mobility-modifying moiety is attached is charged and the mobility-modifying moiety has the opposite charge.

In various embodiments, M may be an oligomer, having monomers of the same or different chemical characteristics, e.g., nucleotides and amino acids. The mobility-modifying moieties may comprise a single type of monomer that provides the different functionalities for oligomerization and that carries a charge. Alternatively, two or more different monomers may be employed. Substituted diols may be used, where the substituents are charged and dibasic acids. Illustrative of such oligomers is the combination of diols or diamino, such as 2,3-dihydroxypropionic acid, 2,3-dihydroxysuccinic acid, 2,3-diaminosuccinic acid, 2,4-dihydroxyglutaric acid, etc. The diols or diamino compounds can be linked by dibasic acids, which dibasic acids include the inorganic dibasic acids indicated above, as well as organic dibasic acids, such as oxalic acid, malonic acid, succinic acid, maleic acid, funaric acid, carbonic acid, etc. Instead of using esters, amides may be used, and amino acids or diamines and diacids may be employed. Alternatively, the hydroxyls or amines may be linked with alkylene or arylene groups.

Pluralities of electrophoretic tags may include oligopeptides for providing the charge, particularly oligopeptides of from 2-6, usually 2-4 monomers, either positive charges, resulting from lysine, arginine and histidine, or negative charges, resulting from aspartic and glutamic acid. Unnatural or synthetic amino acids, such as such as taurine, phosphate substituted serine or threonine, S-α-succinylcysteine, can also be used, as well as co-oligomers of diamines and amino acids, etc. The charge-imparting moiety may also include thioacids and other carboxylic acids having from one to five carbon atoms. The charge imparting moiety may have from 1 to about 30, preferably 1 to about 20, more preferably, 1 to about 10 amino acids per moiety and may also comprise 1 to about 3 thioacids or other carboxylic acids. However, when used with an uncharged sub-region, the charged sub-region will generally have from 1 to about 4, frequently 1 to about 3 amino acids.

By using monomers having multiple charges, a lower number of monomers can be employed to provide for mobility variation with changes in molecular weight. Of particular interest are polyolpolycarboxylic acids having from about two to four of each functionality, such as tartaric acid, 2,3-dihydroxyterephthalic acid, 3,4-dihydroxyphthalic acid, D⁵-tetrahydro-3,4-dihydroxyphthalic acid, etc. To provide for an additional negative charge, these monomers may be oligomerized with a dibasic acid, such as a phosphoric acid derivative to form the phosphate diester. Alternatively, the carboxylic acids can be used with a diamine to form a polyamide, while the hydroxyl groups can be used to form esters, such as phosphate esters, or ethers such as the ether of glycolic acid, etc.

To vary mobility, various aliphatic groups of differing molecular weight may be employed, such as polymethylenes, polyoxyalkylenes, polyhaloaliphatic or aromatic groups, polyols, e.g., sugars, where the mobility will differ by at least about 0.01, more usually at least about 0.02 and more usually at least about 0.5.

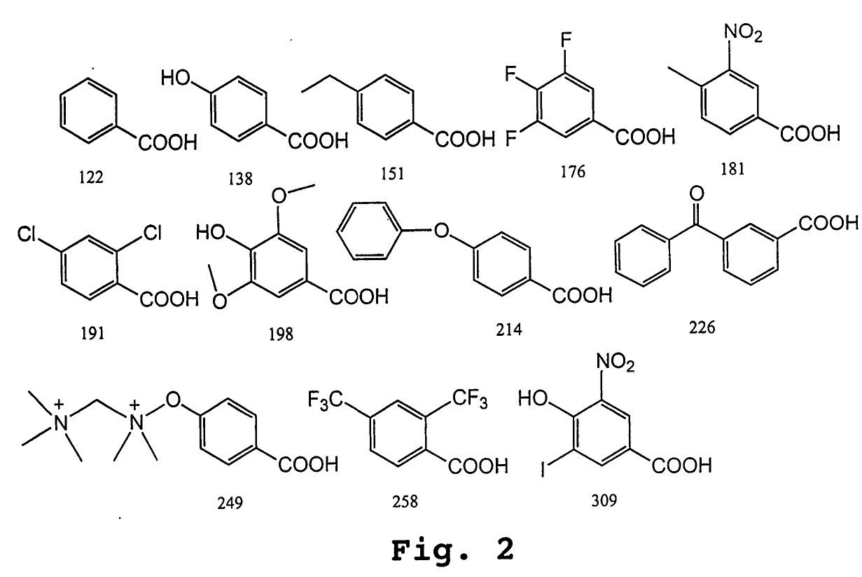

Substituted aryl groups can serve as both mass- and charge-modifying regions (FIG. 2).

Methods of forming selected-length polyethylene oxide-containing chains are well known, see, e.g. Grossman et al., U.S. Pat. No. 5,777,096. It can be appreciated that these methods, which involve coupling of defined-size, multi-subunit polymer units to one another, directly or via linking groups, are applicable to a wide variety of polymers, such as polyethers (e.g., polyethylene oxide and polypropylene oxide), polyesters (e.g., polyglycolic acid, polylactic acid), polypeptides, oligosaccharides, polyurethanes, polyamides, polysulfonamides, polysulfoxides, polyphosphonates, and block copolymers thereof, including polymers composed of units of multiple subunits linked by charged or uncharged linking groups. In addition to homopolymers, the polymer chains used in accordance with the invention include selected-length copolymers, e.g., copolymers of polyethylene oxide units alternating with polypropylene units. Additionally, polypeptides of selected lengths and amino acid composition (i.e., containing naturally occurring or man-made amino acid residues), as homopolymers or mixed polymers may be used.

Various oligomers may be synthesized on a support or produced by cloning or expression in an appropriate host. Conveniently, polypeptides can be produced having only one cysteine (for a thiol functionality), serine/threonine/tyrosine(hydroxyl), aspartic/glutamic acid (carboxyl), or lysine/arginine/histidine (amino), other than an end group, providing a unique functionality which may be differentially functionalized. By using protective groups, a side-chain functionality can be distinguished from a terminal amino acid functionality. Also, by appropriate design, one may provide for preferential reaction between the same functionalities present at different sites on the mobility-modifying moiety. Whether one uses synthesis or cloning for preparation of oligopeptides, is to a substantial degree dependent on the length of the mobility-modifying moiety.

(M, D) moieties can be conveniently constructed from one or more of the same or different common or commercially available linking, cross-linking, and labeling reagents that permit facile assembly, especially using a commercial DNA or peptide synthesizer for all or part of the synthesis. In this aspect, (M, D) moieties are made up of subunits usually connected by phosphodiester and amide bonds. Exemplary precursors that form amide bonds include Fmoc- or Boc-protected amino acid precursors, and derivatives thereof, e.g. as commercially available from AnaSpec, Inc. (San Jose, Calif.). Exemplary precursors that form phosphodiester bonds include various commercially available substituted and protected phosphoramidites, e.g. 6-(4-monomethoxytritylamino)hexyl-(2-cyanoethyl)-(N,N-diisopropyl)-phosphoramidite, (S-trityl-6-mercaptohexyl)-(2-cyanoethyl(N,N-diisopropyl)-phosphoramidite, 5′-fluorescein phosphoramidite, 1,3-bis-[5-(4,4′-dimethoxytrityloxy)pentylamido]propyl-2-[(2-cyanoethyl)-(N,N-diisopropyl)]-phosphoramidite, etc.

Mobility modifiers may also be constructed from such commercially available reagents as succinimidyl trans-4-(maleimidylmethyl)cyclohexane-1-carboxylate (SMCC), succinimidyl 3-(2-pyridyldithio)propionate (SPDP), succinimidyl acetylthioacetate, Texas Red-X-succinimidyl ester, 5- and 6-carboxy tetramethylrhodamine succinimidyl ester, bis-(4-carboxypiperidinyl)sulfonerhodamine di(succinimidyl ester), 5- and 6-((N-(5-aminopentyl)aminocarbonyl)tetramethylrhodamine, succinimidyl 4-(p-maleimidophenyl)butyrate (SMPB); N-γ-maleimidobutyryl-oxysuccinimide ester (GMBS); p-nitrophenyl iodoacetate (NPIA); 4-(4-N-maleimidophenyl)butyric acid hydrazide (MPBH); and like reagents. The above reagents are available from Glen Research (Sterling, Va.), Molecular Probes (Eugene, Oreg.), Pierce Chemical, and like reagent providers. Use of the above reagents in conventional synthetic schemes is well known in the art, e.g. Hermanson, Bioconjugate Techniques (Academic Press, New York, 1996).

In another aspect, (M,D) moieties are constructed from chemical scaffolds used in the generation of combinatorial libraries. For example, the following references describe scaffold compounds useful in generating diverse mobility modifying moieties: peptoids (PCT Publication WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomeres such as hydantoins, benzodiazepines and dipeptides (Hobbs DeWitt, S. et al., Proc. Nat. Acad. Sci. U.S.A. 90: 6909-6913 (1993)), vinylogous polypeptides (Hagihara et al., J. Am. Chem. Soc. 114: 6568 (1992)), nonpeptidal peptidomimetics with a β-D-glucose scaffolding (Hirschmann, R et al., J. Am. Chem. Soc. 114: 9217-9218 (1992)), analogous organic syntheses of small compound libraries (Chen, C. et al., J. Am. Chem. Soc. 116:2661(1994)), oligocarbamates (Cho, C. Y. et al., Science 261: 1303(1993)), peptidyl phosphonates (Campbell, D. A. et al., J. Org. Chem. 59:658(1994)); Cheng et al., U.S. Pat. No. 6,245,937; Heizmann et al., “Xanthines as a scaffold for molecular diversity,” Mol. Divers. 2:171-174 (1997); Pavia et al., Bioorg. Med. Chem. 4:659-666 (1996); Ostresh et al., U.S. Pat. No. 5,856,107; Gordon, E. M. et al., J. Med. Chem. 37:1385 (1994); and the like. Preferably, in this aspect, D is a substituent on a scaffold and M is the rest of the scaffold.

The mobility modifier may also comprise an alkylene or aralkylene group, the latter comprising a divalent aliphatic group having about 1 to about 2 aliphatic regions and about 1 to about 2 aromatic regions, generally benzene, where the groups may be substituted or unsubstituted, usually unsubstituted, comprising from 2 to about 16, more usually 2 to about 12, carbon atoms. Such mobility modifiers may be used to link one or more fluorescers to a monomeric unit, e.g., an amino acid. The mobility-modifying moiety may terminate in a carboxy, hydroxy or amino group, forming an ester or amide upon conjugation. By varying the substituents on the fluorescer(s), one can vary the mass in units of at least about 5 or more, usually at least about 9. To add hydrophilicity, alkyleneoxy groups may be used in place of aralkylene groups.

In some embodiments, the electrophoretic moieties need not be charged, but merely differ in mass. Thus, the same or similar monomers can be used, where the functionalities are neutral or converted to neutral moieties, such as esters and amides of carboxylic acids. Also, the electrophoretic moieties may be varied by isotopic substitution, such as ²H, ¹⁸O, ¹⁴C, etc.

While the charge to mass ratio is an important aspect in differences between e-tag reporters and in particular between mobility modifiers, it is not the only manner by which e-tag reporters may differ from one another. The e-tag reporters may differ by overall topography of the e-tag reporter, i.e., the detection group and the mobility modifier. For example, the mobility modifier may comprise a rigidifier, or substituent that comprises one or more rings. Examples include an aryl moiety, such as, e.g., phenyl, benzyl, naphthyl, and so forth, a cycloalkyl moiety where alkyl is 3 to 20 carbon atoms such as, e.g., cyclopentyl, cyclohexyl and so forth, and the like. Any rigidifier may be employed that imparts a coefficient of drag for the e-tag reporter and, thus, results in a species with separation characteristics that differ from the separation characteristics of other e-tag reporters. Substituted aryl groups can serve as both mass- and charge-modifying regions. Various functionalities may be substituted onto a ring such as an aromatic group, e.g. phenyl, to provide mass as well as charges to the e-tag reporter in addition to rigidification.

B. Linker

The e-tag reporter (M,D) is linked to the target binding moiety (T) by a bond that may be cleavable thermally, photolytically, chemically, or enzymatically. In one embodiment, the linker is a phosphate linkage in an oligonucleotide which is enzymatically cleavable. Numerous other thermally, photolytically or chemically labile linkages are available; see, for example, U.S. Pat. No. 5,721,099. Where detachment of the etag reporter from all or a portion of the target-binding moiety is desired, there are numerous functionalities and reactants which may be used. Conveniently, ethers may be used; substituted benzyl ether or derivatives thereof, e.g benzhydryl ether, indanyl ether, etc. may be cleaved by acidic or mild reductive conditions. Alternatively, one may employ beta-elimination, where a mild base serves to release the reporter. Acetals, including the thio analogs thereof, may be employed, where mild acid, particularly in the presence of a capturing carbonyl compound, is used for cleavage. Reaction of an alcohol moiety with formaldehyde and HCl forms an α-chloroether, which may then be coupled with a hydroxy functionality to form an acetal, which is acid labile. Various photolabile linkages may be employed, such as o-nitrobenzyl, 7-nitroindanyl, 2-nitrobenzhydryl ethers or esters, etc.

Various other cleavable functionalities include: silyl groups, cleavable with fluoride, oxidation, acid, bromine or chlorine; o-nitrobenzyl (photocleavable); catechols, cleavable with cerium salts; olefins, cleavable with ozone, permanganate or osmium tetroxide; sulfides, cleavable with singlet oxygen or enzyme catalyzed oxidative cleavage with hydrogen peroxide, where the resulting sulfone can undergo elimination; furans, cleavable with oxygen or bromine in methanol; tertiary alcohols, ketals and acetals, cleavable with acid; α- and β-substituted ethers and esters, cleavable with base, where the substituent is an electron withdrawing group, e.g., sulfone, sulfoxide, ketone, etc.

For a survey of additional cleavable linkages, see, for example, Greene and Wuts, Protective Groups in Organic Synthesis, 2^{nd} ed. Wiley, 1991. The versatility of the various systems that have been developed allows for broad variation in the conditions for attachment of the e-tag entities.

The linker L may include alkylene and substituted alkylenes, oxyalkylene and polyoxyalkylene, particularly alkylene of from 2 to 3 carbon atoms, arylenes and substituted arylenes, polyamides, polyethers, polyalkylene amines, etc. It may include charge-imparting moieties such as, amino acids, tetraalkylammonium, phosphonium, phosphate diesters, carboxylic acids, thioacids, sulfonic acids, sulfate groups, or phosphate monoesters.

Of particular interest for L is to have two sub-regions, a common charged sub-region, which will be common to a group of e-tag moieties, and a varying uncharged, a non-polar or polar sub-region, that will vary the mass/charge ratio. This permits ease of synthesis, provides for relatively common chemical and physical properties and permits ease of handling.

Conjugates of particular interest for use as reporter groups are represented by the formula:
D′-L-(-Z′)ᵥ-T′
wherein D′ is a fluorescer; L is a linker as defined above; T′ is a nucleoside, nucleotide or nucleotide triphosphate, or a functionality for linking to a nucleoside base; Z′ is an organic moiety comprising an interactive functionality as described further below; and v′ is 1 to about 10, preferably 2 to about 8, and more preferably 3 to 6. Z′ is adjacent the cleavable linkage and positioned in the electrophoretic probe such that the interactive functionality does not form part of the released reporter group.

C. Synthesis of Oligonucleotide-Based Probes

Synthesis of electrophoretic probes comprising nucleotides can be easily and effectively achieved via assembly on a solid phase support during probe synthesis, using standard phosphoramidite chemistries. In one approach, the e-tag probe is constructed sequentially from a single or several monomeric phosphoramidite building blocks (one containing a dye residue), which are chosen to generate tags with unique electrophoretic mobilities based on their mass to charge ratio. The e-tag probe is thus composed of monomeric units of variable charge to mass ratios bridged by phosphate linkers.

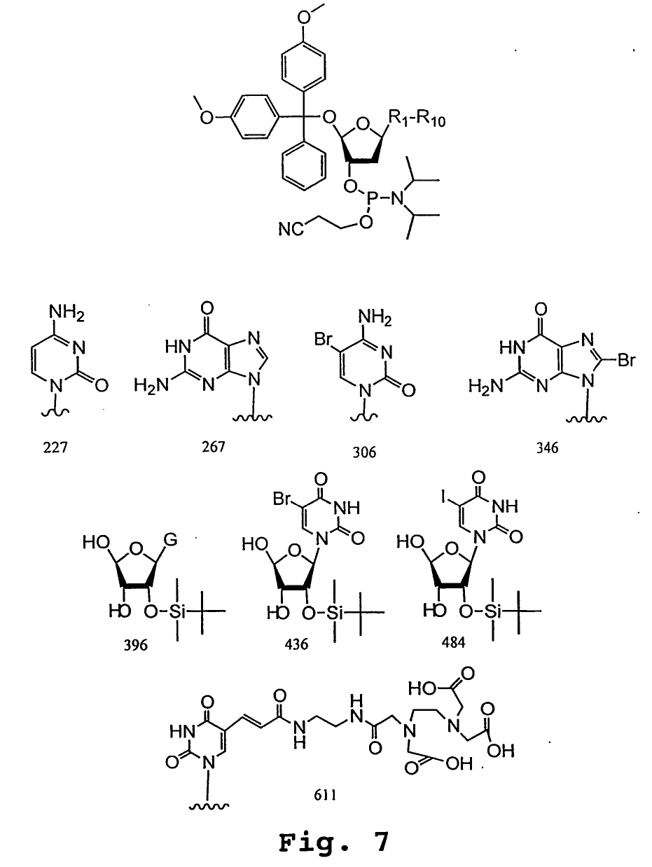

The penultimate coupling during probe synthesis is initially carried out using commercially available modified (and unmodified) phosphoramidites. FIG. 7 illustrates possible structural variations for mobility-modified nucleic acid phosphoramidites that can be employed at the penultimate coupling during e-tag probe synthesis on a standard DNA synthesizer.

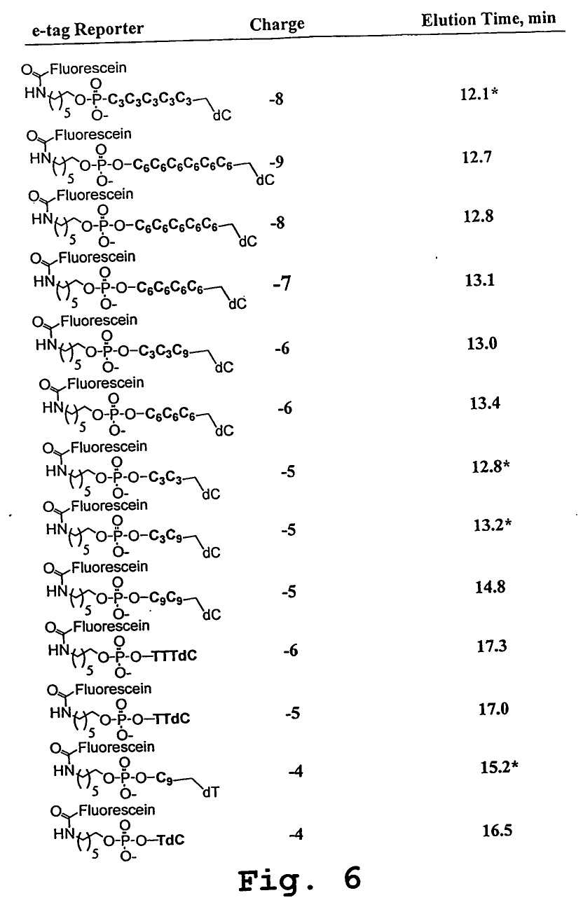

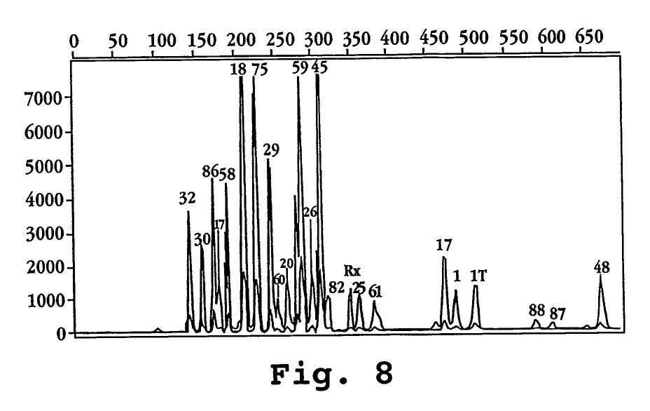

FIG. 6 illustrates predicted and experimental (*) elution times of various nucleotide-containing e-tag reporters. C₃, C₆, C₉, and C₁₈ represent phosphoramidite spacers commercially available from Glen Research, Sterling Va. The units are derivatives of N,N-diisopropyl-O-cyanoethyl phosphoramidite, which is indicated by “Q”. C₃ is DMT (dimethoxytrityl)oxypropyl Q; C₆ is DMToxyhexyl Q; C₉ is DMToxy(triethyleneoxy) Q; C₁₂ is DMToxydodecyl Q; C₁₈ is DMToxy (hexaethyleneoxy) Q. This set of e-tag reagents generates a contiguous spectrum of signals upon electrophoretic separation, one eluting after another with none of them coeluting (FIG. 8).

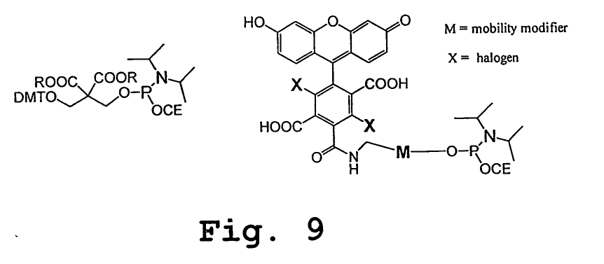

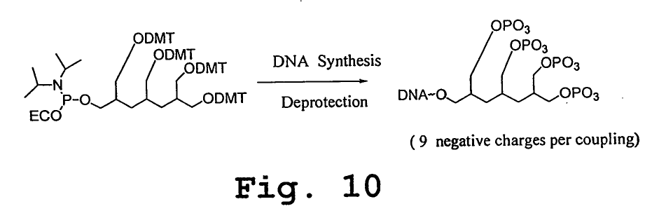

Use of dicarboxylate phosphoramidites (FIG. 9, left) allows for the addition of 3 negative charges per coupling of monomer, providing highly charged tags with short elution times. A variety of fluorescein derivatives (FIG. 9, right) allow the dye component of the tag to carry a higher mass than standard fluorescein. Polyhydroxylated phosphoramidites (FIG. 10) in combination with a common phosphorylation reagent enable the synthesis of highly phosphorylated tags. Combinations of these reagents with other mass modifier linker phosphoramidites allow for the synthesis of tags with short elution times.

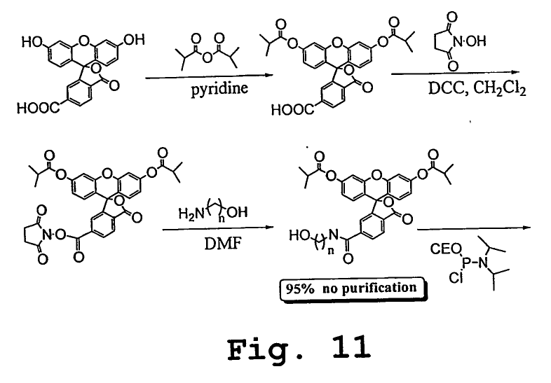

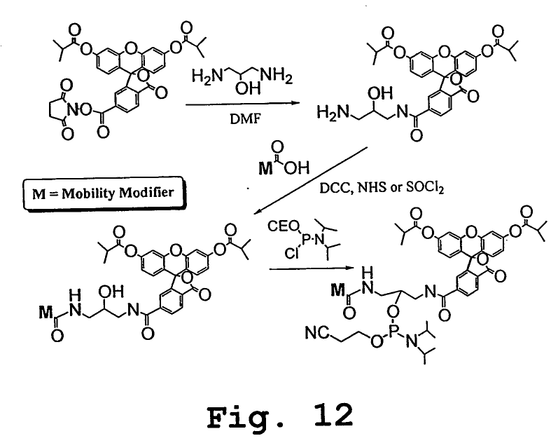

The etags are linked to oligonucleotides in a manner similar to that for labels in general, by means of linkages that are enzymatically cleavable. One exemplary synthetic approach is outlined in FIG. 11. Starting with commercially available 6-carboxy fluorescein, the phenolic hydroxyl groups are protected as esters, generally by reaction with an anhydride. The crude material is then activated in situ via formation of an N-hydroxy succinimide ester (NHS-ester) using DCC as a coupling agent. The DCU byproduct is filtered away and an amino alcohol is added. Many amino alcohols are commercially available, some of which are derived from reduction of amino acids. Only the amine is reactive enough to displace N-hydroxy succinimide. Upon standard extractive workup, a 95% yield of product is obtained. This material is phosphitylated to generate the phosphoramidite monomer (FIG. 11). For the synthesis of additional e-tag probes, a symmetrical bis-amino alcohol linker is used as the amino alcohol (FIG. 12). As such, the second amine can then be coupled with any of a multitude of carboxylic acid derivatives (exemplified by several possible benzoic acid derivatives shown in FIG. 2) prior to the phosphitylation reaction. Using this methodology, hundreds, even thousands of e-tag moieties with varying charge to mass ratios can easily be assembled during probe synthesis on a DNA synthesizer using standard chemistries.

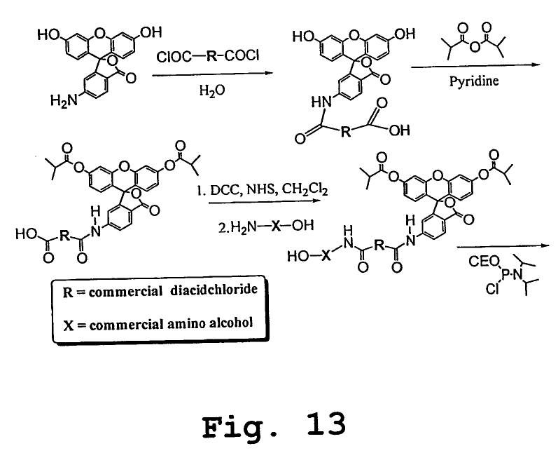

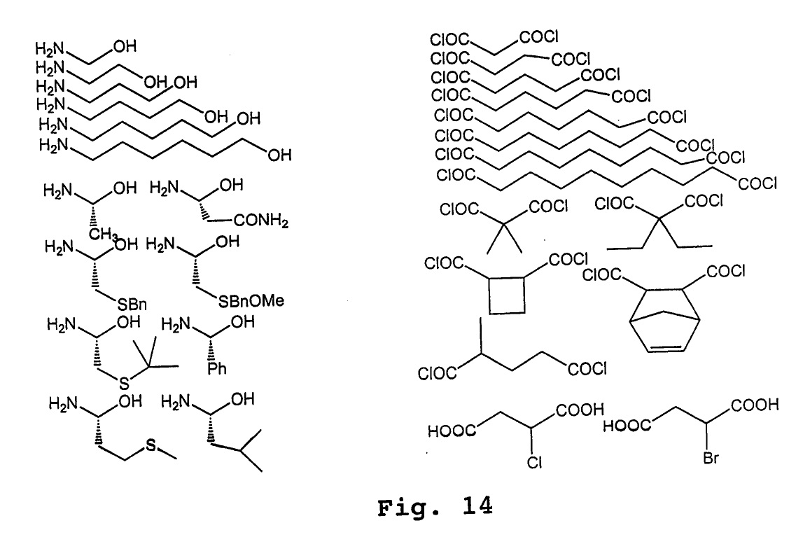

Alternatively, e-tag reagents are accessed via an alternative strategy that uses 5-aminofluorescein as starting material (FIG. 13). Addition of 5-aminofluorescein to a great excess of a diacid dichloride in a large volume of solvent allows for the predominant formation of the monoacylated product over dimer formation. The phenolic groups are not reactive under these conditions. Aqueous workup converts the terminal acid chloride to a carboxylic acid. This product is analogous to 6-carboxyfluorescein, and using the same series of steps is converted to its protected phosphoramidite monomer (FIG. 13). There are many commercially available diacid dichorides and diacids, which can be converted to diacid dichlorides using SOCl₂ or acetyl chloride. This methodology is highly attractive in that a second mobility modifier is used. As such, if one has access to 10 commercial modified phosphoramidites and 10 diacid dichlorides and 10 amino alcohols there is a potential for 1000 different e-tag moieties. There are many commercial diacid dichlorides and amino alcohols (FIG. 14). These synthetic approaches are ideally suited for combinatorial chemistry.

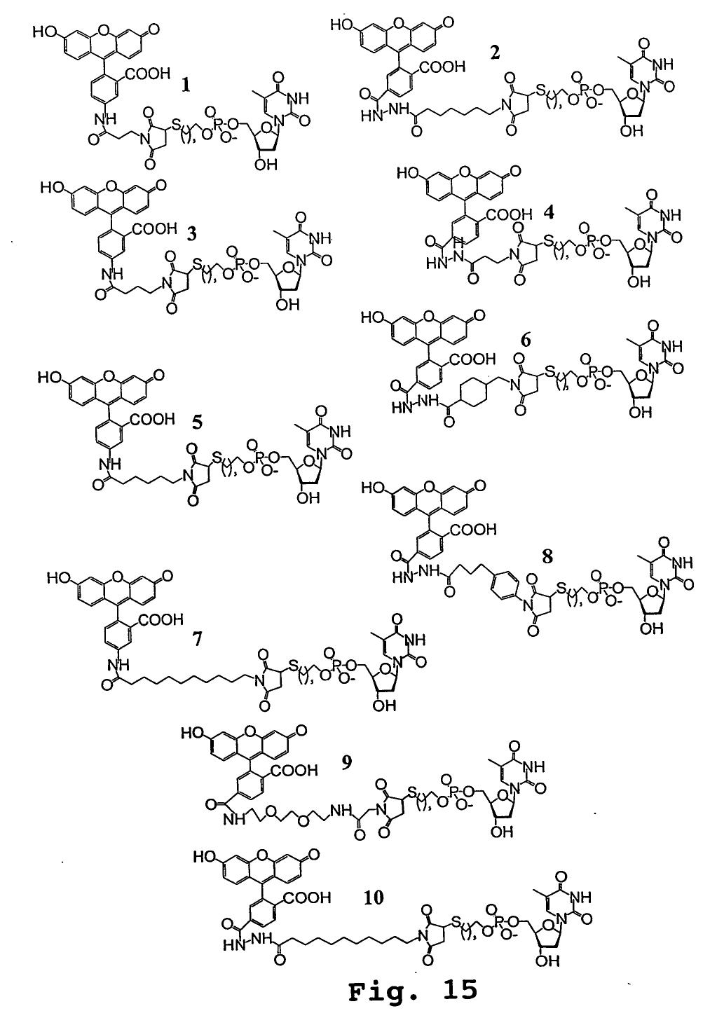

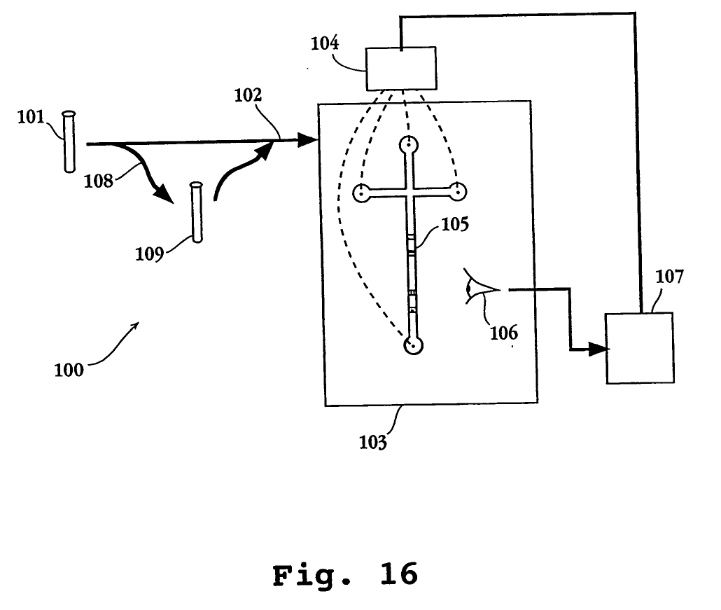

A variety of maleimide-derivatized e-tag groups have also been synthesized. These compounds were subsequently bioconjugated to 5′-thiol derivatized DNA sequences and subjected to the 5′-nuclease assay. The reporter groups formed upon cleavage are depicted in FIG. 15.

For a peptide-binding compound, a variety of functionalities can be employed, much as with the oligonucleotide functionality, although phosphoramidite chemistry may only occasionally be appropriate. Thus, the functionalities normally present in a peptide, such as carboxy, amino, hydroxy and thiol, may be the targets of a reactive functionality for forming a covalent bond. For example, with Fab′ fragments specific for a target compound, a thiol group will be available for using an active olefin, e.g. maleimide, for thioether formation. Where lysines are available, one may use activated esters capable of reacting in water, such as nitrophenyl esters or pentafluorophenyl esters, or mixed anhydrides as with carbodiimide and half-ester carbonic acid.

For separations based on sorption, adsorption and/or absorption, the nature of the e-tag reporters to provide for differentiation can be relatively simple. By using differences in composition, such as aliphatic compounds, aromatic compounds and halo derivatives thereof, the determinations may be made with gas chromatography, with electron capture or negative ion mass spectrometry, when electronegative atoms are present. In this way hydrocarbons or halo-substituted hydrocarbons may be employed as the e-tag reporters bonded to a releasable linker. See, U.S. Pat. Nos. 5,565,324 and 6,001,579, which are specifically incorporated by reference as to the relevant disclosure concerning cleavable groups and detectable groups.

D. Interactive Functionality

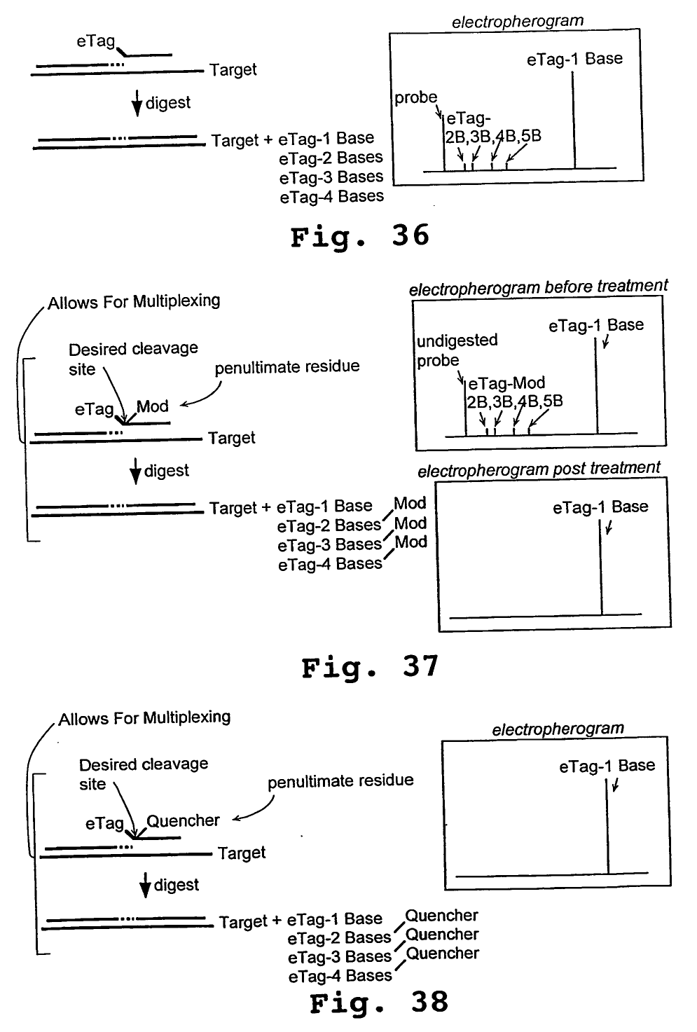

To enhance the multiplexing capabilities of e-tag technology, it is important that only one detectable product be generated per target. Even small amounts of additional signal generating products generated during the assay reduce multiplexing abilities. For example, during 5′-nuclease-based assays (such as TaqMan), one major product is generated by cleavage by polymerase at the predicted terminal phosphate linkage. However, small amounts of additional cleavage products are generated via cleavage by the enzyme at phosphate sites further removed from the major cleavage site. Additionally, a vast majority of the probe containing the e-tag moiety is left intact. Other interfering species may occur as a result of degradation of the starting material, contamination, aberrant cleavage, or other nonspecific degradation products of the target binding sequence. It is desirable for this unreacted material to be removed as well. FIG. 36 depicts typical products generated by a 5′-nuclease assay and the resulting electropherogram generated. This situation allows for only low level of multiplexing.

The methodology described herein permits removal of signal from all unwanted cleavage products, as well as any uncleaved probe. Accordingly, probes are modified as described herein to provide for isolation of released reporter group (or, more specifically, signal from released reporter group) from intact probe and other components. The invention may be understood with reference to introduction of a uniquely functionalized nucleic acid residue at the 5′ penultimate position of a probe sequence. This is by way of illustration and not limitation since the invention has application to electrophoretic probes other than polynucleotide electrophoretic probes.

In one general embodiment of the target-binding moiety for use in detection of nucleic acid targets, Tⱼ is an oligonucleotide target-binding moiety. In such cases, Tⱼ comprises a sequence of nucleotides Uᵢ connected by intersubunit linkages:
5′-U₁—U₂—U₃—U₄—U₅—U₆—Uᵢ 3′
where — corresponds to intersubunit linkages B_{i,i+1}. When cleavage to release the e-tag reporter occurs between the first and second 5′ nucleotides (i.e. between U₁ and U₂ above), U₁ is considered the 5′ nucleotide of the target-binding moiety (as in the representation above), and cleavage occurs within this moiety, that is, at a nuclease-susceptible bond between the first and the second nucleotides of the target moiety (between U₁ and U₂, above). In this representation, the bond between the first and second nucleotides (B_{1,2} in the above nomenclature) is the site of cleavage and all downstream bonds are represented by B_{i,i+1}, where “i” is 2 or greater. The target binding moiety may include one or more nuclease-resistant linkages adjacent to the nuclease-susceptible linkage U₁—U₂. In accordance with the invention, an interactive functionality may be bound to the penultimate nucleotide (U₂).

As described above, an organic moiety comprising an interactive functionality is positioned adjacent the cleavable linkage in the electrophoretic probe, such that the interactive functionality does not form part of the released reporter group upon release from the electrophoretic probe.

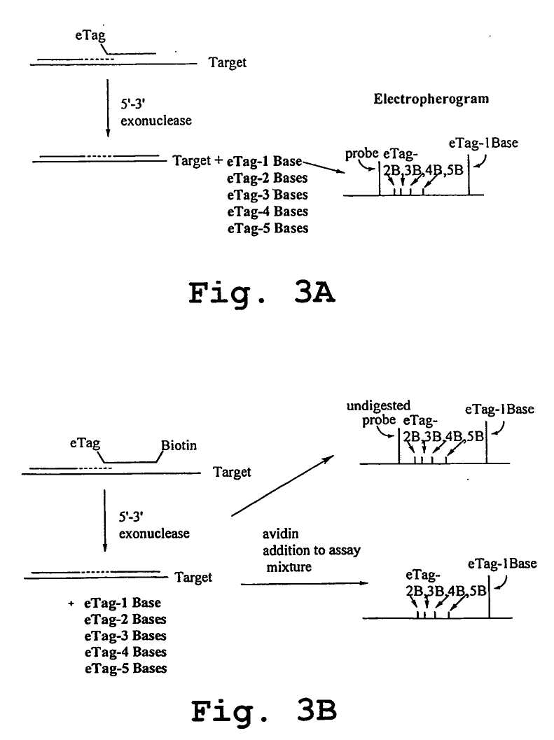

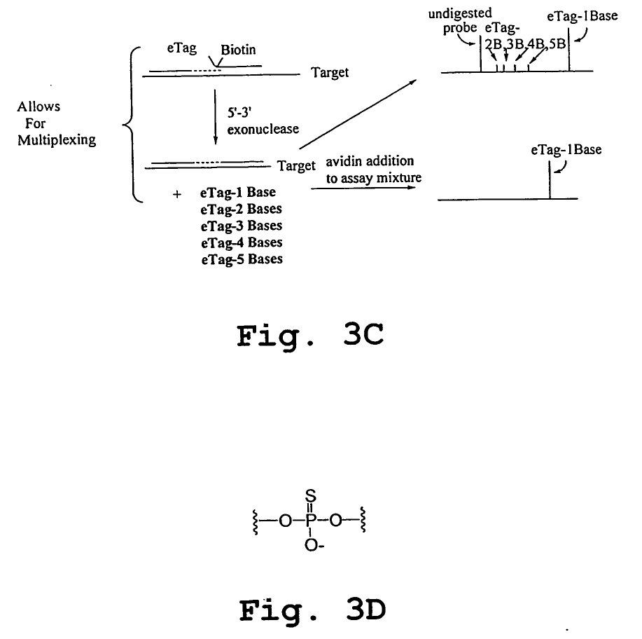

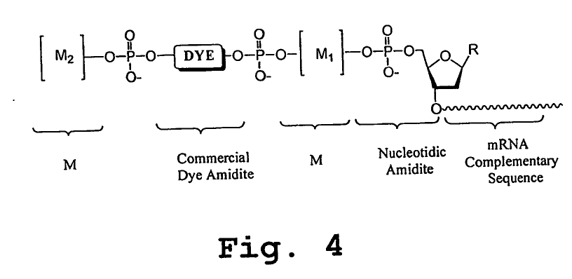

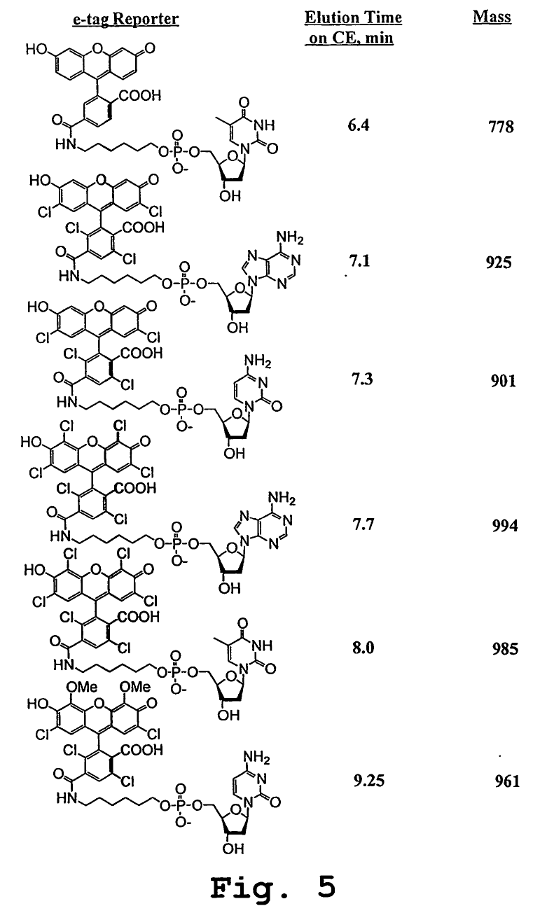

FIGS. 3A-C provide a schematic illustration of the advantages of this method, in an assay employing a nucleotide target and a 5′ exonuclease. In this assay, a cleavage structure is formed comprising a target oligonucleotide, primer sequence, and etag containing probe sequence. In accordance with the assay method, when probe binds specifically to target oligonucleotide to form the cleavage structure, the probe is susceptible to cleavage by a 5′-exonuclease at the nucleotide containing the etag reporter group.

In FIG. 3A, where no capture agent is used, the electropherogram includes signals for uncleaved probe, as well as for several other extraneous cleavage products (cleaved at positions other than that immediately adjacent the etag reporter group).

In FIG. 3B, an interactive functionality (a biotin group) is included at the 3′terminus of the probe. While this approach allows intact probe to be removed from the cleavage mixture by addition of avidin, as shown in the electropherogram, interfering signals from extraneous cleavage products, which do do not contain the biotin group, are still present.

In FIG. 3C, the interactive moiety (biotin) is positioned at the penultimate nucleotide of the probe, immediately adjacent the cleavable reporter group. Accordingly, contacting the cleavage mixture with avidin (capture reagent) removes extraneous cleavage product as well as intact probe, and produces an electropherogram having only the reporter group signal (FIG. 3C).

D1. Quenching Moiety

In one embodiment, the interactive functionality interacts with the detection moiety to prevent the detection moiety from generating a signal in the intact probe. One such moiety is a quencher, which quenches the signal from the detection moiety in a proximity-dependent manner. When the e-tag reporter is cleaved, the quencher is no longer in close proximity to the detection moiety, which thus regains its ability to generate a signal. In this embodiment, an additional step for removal of unwanted species is not necessary. All of the unwanted species, e.g., unreacted starting reagents, cleaved probe, and extraneous cleavage products, generally retain quencher in close proximity to the fluorescent moiety. Accordingly, these unwanted species remain undetected during electroseparation analysis, without the need for a separation step.

A diverse number of quenchers or energy acceptors may be employed. The energy acceptor is able to absorb energy from the first fluorescent molecule in a proximity-dependent mannter. Energy acceptors are chromophores having substantial absorption higher than 310 nm, normally higher than 350 nm, and preferably higher than about 400 nm. Generally, they are fluorescent compounds, but they may have weak or no fluorescence and still be useful. One group includes xanthene dyes, which include the fluoresceins, derived from 3,6-dihydroxyo-phenyl-xanthhydrol, and rhodamines, derived form 3,6-diamino-9-phenylxanthhydrol. Another group includes chelates of fluorescent lanthanides such as Eu and Sm. Another group includes the naphthylamines, such as, e.g., 1-anilino-8-naphthalene sulfonate, 1-dimethylaminonaphthyl-5-sulfonate. Another group includes natural proteinaceous pigments such as the phycobiliproteins and green fluorescent proteins.

Energy acceptors that are non-fluorescent can include any of a wide variety of azo dyes, cyanine dyes, 4,5-dimethoxyfluorescein, formazans, indophenols and the like. A further discussion of fluorescers and quenchers may also be found in U.S. Pat. Nos. 4,261,968, 4,174,384, 4,199,983 and 3,996,345, the relevant disclosures of which are incorporated herein by reference.

In the intact electrophoretic probe, the fluorescer and quencher are in sufficient proximity that the fluorescence of the fluorescer is substantially quenched. The fluorescer and quencher are on opposing sides of the cleavable linkage. Fluorescers and quenchers may be incorporated into the e-tag probe by virtue of a bond or linking group, using method suitable for incorporating a label into a probe. For oligonucleotide probes, the fluorescer or the quencher may be incorporated during synthesis, using nucleoside reagents that comprise the fluorescer or quencher.

A specific embodiment is depicted in FIG. 38. Probes containing a quencher residue at the penultimate position result in a single cleavage product (the desired product) being detected during capillary electrophoretic analysis. All undesired products, as well as intact probe, still contain quencher, which renders them undetected during electrophoresis.

D2. Isolation via Capture Auent

As discussed above, with references to FIGS. 3A-C, the interactive functionality may also act by enabling separation of the released detection group and mobility modifier (i.e. the released etag reporter) from the residue or the electrophoretic probe. In this embodiment, an additional capture reagent is included that binds to the interactive functionality and enables separation of the various components carrying the detection group.

After cleavage of the reporter group from the probe has taken place in an assay, and prior to electrophoretic analysis, a capture agent is added to bind with the interactive functionality on the probe. Preferably, the interactive functionality is at the penultimate residue of a probe, and the cleavable reporter group on the terminal residue, such that all undesired cleavage products, as well as the intact probe, will contain the interactive functionality and will bind to the capture agent. The resulting species, when resulting from chelation, are positively charged and are thus not injected during capillary electrophoretic analysis.

Alternatively, the interactive functionality may be present at a position other than the penultimate position, where the ultimate linkage is modified to be nuclease resistant, so that cleavage is directed to the penultimate linkage.

In any of the interactive functionality—capture agent systems described herein, the components are generally interchangeable; that is, either may be employed as the interactive funcationality on the probe, and the other as the capture agent in the assay mixture. For example, as described below, a boronic acid moiety may be incorporated into the probe, and a diol-containing reagent used as the capture agent, or vice versa.

In one embodiment, the interactive functionality and the capture reagent are a chelator and a chelator substrate, or vice versa In one embodiment, the chelator substrate (also referred to as a “chelating moiety”) comprises a functionality that includes at least two groups such as, for example, hydroxy, thio, amino, substituted (e.g. alkyl or aryl) amino, carbonyl, oxo, carboxyl, carboxamide, N-hydroxy amide, and the like, or combinations thereof. Specific functionalities include, for example, 1,2-diols, 1,3-diols, 1,2-aminoalcohols, 1,3-aminoalcohols, 1,2-hydroxy acids, 1,3-hydroxy acids, 1,2-diketones, 1,3-diketones, 1,2-hydroxy acid amides, 1,3-hydroxy acid amides, dioximes, and so forth.

Preferably, the groups in the interactive functionality are in a cis and/or coplanar relationship. The cis and/or coplanar relationship may result from the groups being disposed on an unsaturated bond, such as a carbon-carbon double bond or a ring having a geometry that results in a cis relationship, such as an aromatic ring or rigid aliphatic ring, including fused systems. The coplanar relationship may be found, for example, in a fused ring system where each functionality is disposed on a different ring in, for example, a 1,8-naphthyl system.

In one exemplary embodiment, the interactive functionality is a boronic acid complexing moiety, and the capture reagent is a boronic acid, or vice versa Preferably, the boronic acid complexing moiety has a pKa in the range of about 3-11, preferably about 4-10. The boronic acid complexing moieties generally have one or more, usually at least 2 or more, groups, as disclosed above, that can complex or react with a boronic acid group to form a boronic acid ester or diester. The functionalities may be part of an organic moiety having about 6 to about 100 atoms, preferably about 5 to about 50 atoms, preferably selected from the group consisting of carbon, oxygen, nitrogen, sulfur. The organic moiety may have from about one to about 10 of the aforementioned groups.

Specific examples of organic moieties having one or more of the aforementioned functionalities are, by way of illustration and not limitation, ortho-hydroxybenzenes (catechols), ortho-hydroxy benzohydroxamic acids, ortho-hydroxybenzoic acids, ortho-hydroxybenzamides, imidazolyl phenols, dihydroxyfumaric acids, hydroxy pyridine aldehydes, and hydroxyanthraquinone dioximes. Examples of particular compounds that may be incorporated into synthetic biopolymers or biopolymer precursors include catechol, salicylamide, N-phenylsalicylamidine, 2-(4′imidazolyl)phenol, 1,8-dihydroxynaphthalene, dihydroxyfumaric acid, salicylaldehyde, 3-hydroxypyridine-4-aldehyde, pyridoxamine, 1-hydroxyanthraquinone dioxime, and disalicylimide.

An appropriatecapture agent for a probe bearing a cis-diol interactive functionality is a boronic acid moiety. The boronic acid moiety is typically a substituted boronic acid, wherein the substituent is an organic moiety, either aliphatic or aromatic, having about 2 to about 100 atoms, selected from the group consisting of carbon, oxygen, nitrogen, and sulfur; usually the organic moiety has from about 2 to about 60 carbon atoms. Such a substituted boronic acid may be represented as R—B(OH)₂, where R is the organic moiety.

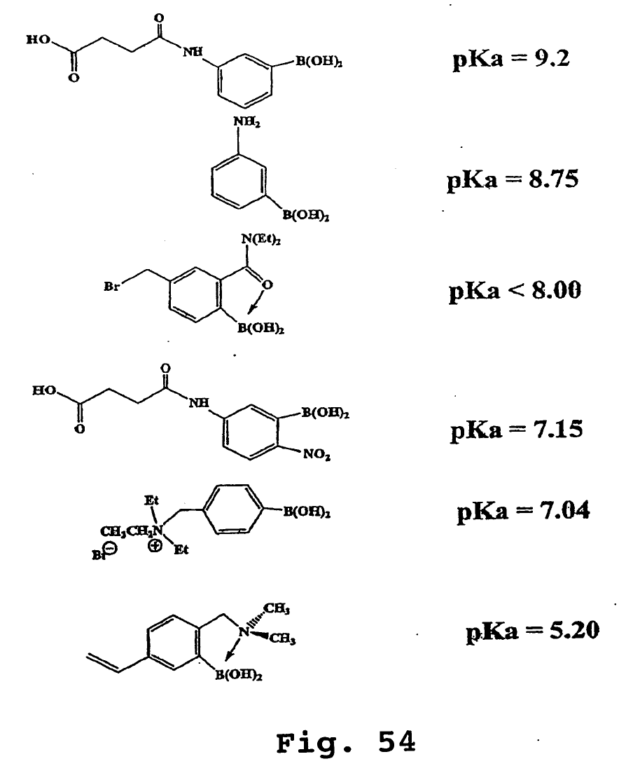

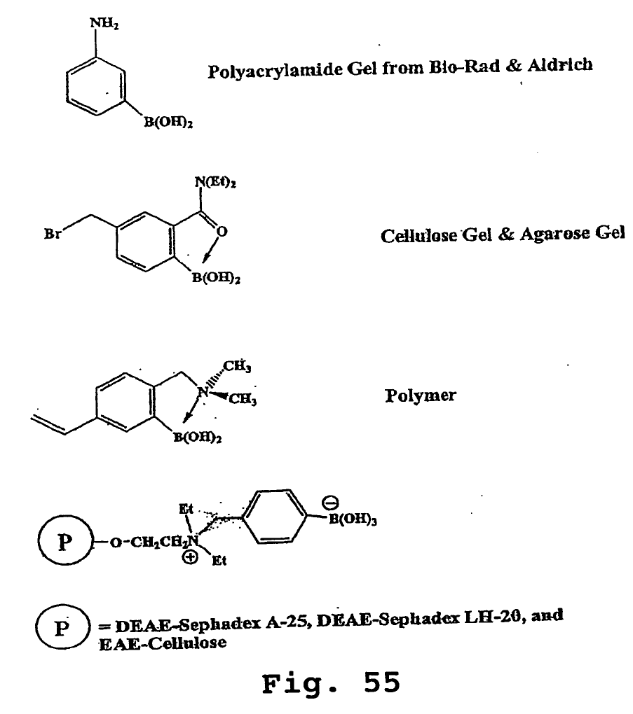

An important consideration regarding the boronic acid moiety is its acidity. In general, the higher the acidity of the boronic acid moiety, the better is the ability to complex with the boronic acid complexing moiety and to to separate biomolecules under physiological conditions, e.g. at a pH of about 7.4. Desirably, the pKa of the boronic acid moiety is <11, preferably <9. Accordingly, substituents on the boron that enhance the acidity over that of boronic acid are preferred. Aromatic substituents on the boron are preferred; to enhance acidity, the aromatic substituent preferably contains one or more electron-withdrawing groups such as nitro, cyano, carboxyl, keto, and the like. Specific boronic acid moieties include, by way of illustration and not limitation, phenyl boronic acid and (3-aminophenyl) boronic acid. Other examples of specific phenylboronic acids are depicted in FIGS. 54-55, and still other examples may be found in U.S. Pat. Nos. 5,623,055, 5,876,938, 6,013,783 and 5,831,045, the relevant disclosures of which are incorporated herein by reference.

In another approach similar to that discussed above, the chelator substrate comprises a dithiol, and the chelator is an arsenic-containing moiety. As above, either may be the capture agent and the other the interactive functionality.

To assist in the separation of unwanted materials from the materials of interest, it is preferable that a support be employed comprising the capture agent. Examples of supports, by way of illustration and not limitation, include polysaccharides, such as dextran, aminodextran, cellulose, agarose; etc., polyacrylamide, polymethylmethacrylate, silica, glass, and the like, or a polymer that includes a capture agent, such as a boronic acid derivative, as an integral part of the polymer backbone. A polysaccharide support such as dextran can itself functions as a complexing moiety for a probe containing, for example, a boronic acid moiety as the interactive functionality.

In various embodiments, the support is a boronate gel, such as, for example, Affi-Gel 601® (BioRad Laboratories, Richmond, Calif.), polyacrylamide bulk gel (Aldrich Chemical Co., Milwaukee, Wis.), agarose bulk gel (Amicon Corp., Danvers, Mass.), polymethylmethacrylate bulk gel (Toyo-Soda Corp., Tokyo, Japan), or agarose or polyacrylamide bulk gel (Pierce Chemical Co., Rockford Ill.). Commercially available boronate supports include a pre-packed SPE silica tube from Analytichem Intl., Harbor City, Calif., a high performance ligand affinity chromatography (HPLAC) column, either agarose or polyacrylamide, from Pierce Chemical Co., or polymethylmethacrylate from Toyo-Soda Corp. Structures of some typical commercially available boronate affinity matrices are shown in FIG. 55. A boronate support can also be prepared by linking a boronic acid moiety to a preformed support. In another approach, a boronic acid moiety may be incorporated into a polymeric support during the polymerization step.

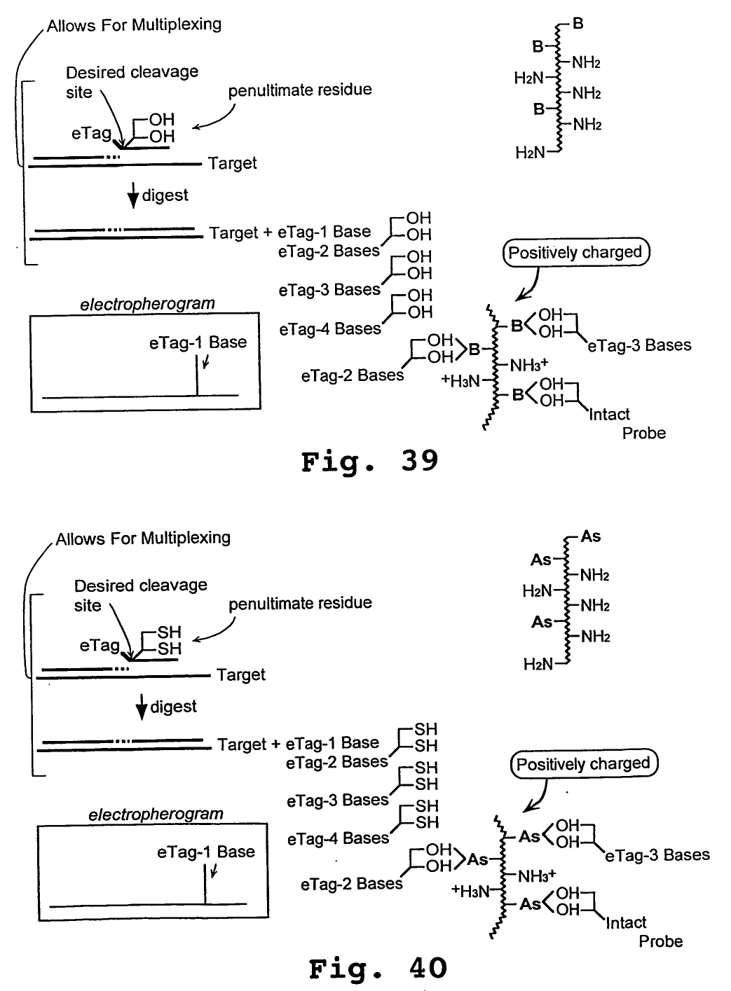

FIG. 39 depicts an embodiment of the assay in which the probes contain a diol-modified residue (interactive functionality) at the penultimate position, allowing all undesired cleavage products, as well as intact probe, to be removed via addition of boronated aminodextran (capture agent). As discussed above, a support is preferably employed comprising the capture agent.

When the capture agent or interactive functionality comprises a boronic acid moiety, complexing is effectively carried out in a buffered medium, usually an aqueous buffered medium, at a pH of about 7.5-10, preferably about 8.5-9.5. Particular buffers that may be employed are those that increase the retention volume of the support, such as HEPES buffer. Additives that increase the retention volume of the support, such as NaCl, MgCl₂, EDTA, piperidine, and the like, may also be included.

FIG. 40 depicts an embodiment of the assay in which the probes contain a dithiol-modified residue (interactive functionality) at the penultimate position, and arsenic-derivatized aminodextran is used as the capture agent.

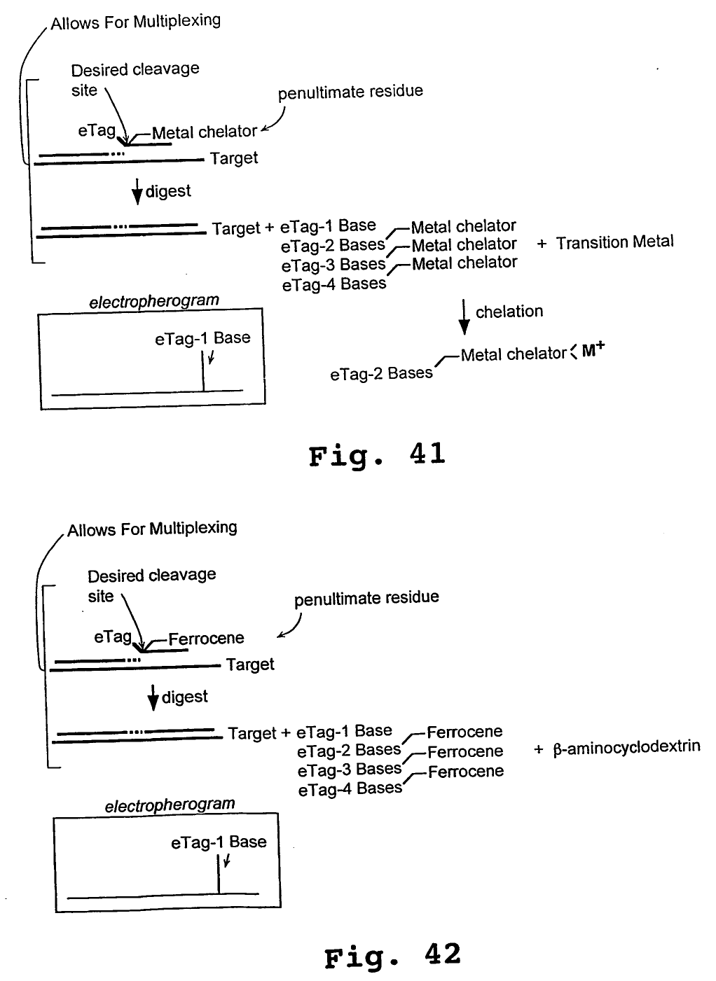

The capture agent or interactive functionality may be a transition metal (e.g. Ni, Fe) chelator. The chelator usually comprises about 2 to about 4 metal coordinating sites, comprising oxygen, nitrogen, sulfur, and the like. Well known examples include bis-imidazoles and acetoacetates. In a particular embodiment, illustrated in FIG. 41, the transition metal chelator is incorporated into the electrophoretic probes, and the support is particulate transition metal, which is contacted with the reaction mixture.

In another embodiment, the interactive functionality is a ferrocene moiety, which may be incorporated into the electrophoretic probe by techniques known in the art. The chelator may be a moiety that comprises an amino group, e.g. a β-aminocyclodextran support. FIG. 42 depicts a ferrocene-based “cleanup” of a 5′-nuclease assay in accordance with this embodiment.

The capture agent-interactive functionality pair may be a ligand and anti-ligand, or receptor pair, or other specific binding pair. Examples include antigen-antibody, biotin-avidin, hormone-hormone receptors, nucleic acid duplexes, IgG-protein A, complementary polynucleotide pairs such as DNA-DNA, DNA-RNA, protein-nucleic acid complexes, and the like. Any of the aforementioned substances may be incorporated into electrophoretic probes and/or made part of a support as discussed herein. Desirably, the capture agent is positively charged, as in the case of avidin, or is modified to have a positive charge by the addition of positively charged groups such as ammonium or basic amino acids.

In one embodiment, an oligonucleotide sequence (which does not hybridize with any assay target) is attached to the electrophoretic probe, and a support gaving bound thereto a hybridizable oligonucleotide sequence is employed as the capture agent. Ligands and receptor pairs that may be used in a similar manner include biotin/streptavidin, digoxin/antidigoxin, etc. For example, after a 5′ nuclease assay employing probes having biotin incorporated as the interactive functionality, avidin is added to the assay mixture (see e.g. Examples 11, 13). Avidin binds to the biotin attached to the detection probe and its degradation products. Avidin is positively charged, while the cleaved electrophoretic tag is negatively charged. Thus the separation of the cleaved electrophoretic tag from uncleaved probe and degradation products is easily achieved by using conventional separation methods. Alternatively, the capture agent receptor may be bound to a solid support as described above, and separated achieved by physical separation or centrifugation, dialysis, etc.

D3. Incorporation of Interactive Functionality into Probes

In general, the interactive functionalities are incorporated into the electrophoretic probe in a manner and at a position such as not to interfere with the binding capabilities and related properties of the probe. The interactive functionalities may be incorporated into the e-tag probe by reaction of one or more functionalities on the e-tag probe backbone with an appropriate functionality on a reagent comprising the interactive functionality. Alternatively, one or more monomers used to prepare an e-tag probe may be treated to incorporate the interactive functionality.

The following discussion is directed to oligonucleotide probes and the nucleoside monomers that form the oligonucleotides. In one embodiment, an interactive functionality, such as a cis-diol functionality, is incorporated at the penultimate nucleotide of an oligonucleotide reagent.

An organic moiety comprising the interactive functionality may be incorporated into e.g. the sugar moiety or the base moiety of a nucleoside. An example of a nucleoside that may be employed is represented by the following formula. In this case, the interactive functionality, represented by the various embodiments of Y, is included in the base moiety (R₄) or the 2′ position of the sugar moiety (R₃).

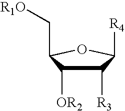

In the above formula, R¹ is H, dimethoxytrityl (DMT), triphosphate ester, diphosphate ester, or monophosphate ester;
R² is H or phosphoramidite;
R³ is H, OH, ODMT or OX(Y)ₚ;
and R⁴ is one of:

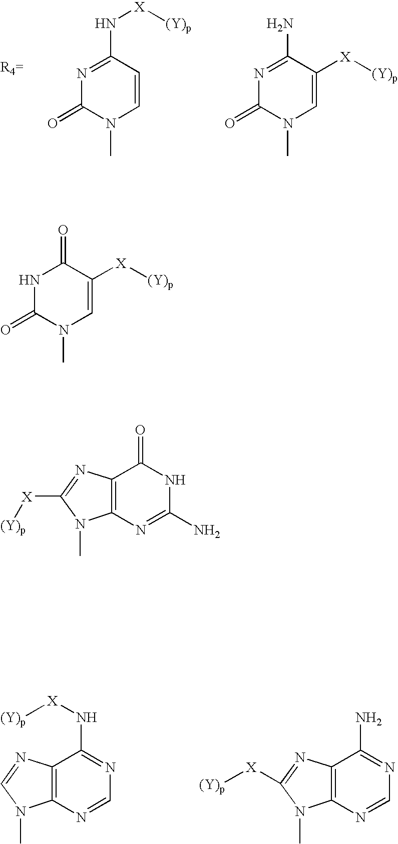

In R⁴, p is 0 to 3, usually 1 to 3, and p in at least one of R³ or R⁴ is 1. X is a bond or a linking group, and Y is Ya or Yb, as shown below:

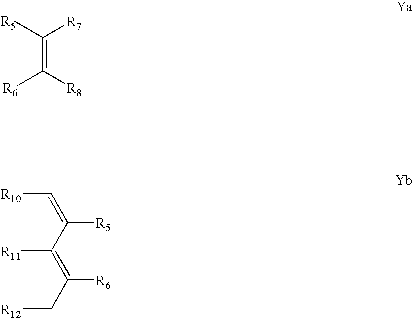

In Ya, R⁵ and R⁶ are independently hydroxy, amino, substituted amino, carbonyl (as in keto, aldehyde and the like), carboxamide, or N-hydroxy amide, where any of these can include a protecting group such as, for example, acetyl, ethyl, isobutyryl, or t-butyryl. R⁷ and R⁸ are independently carboxy or cyano or together form one or more rings, usually 1 to 3 rings, which are usually fused when more than one ring, wherein each of the rings may comprise 5 to 10 atoms, usually 5-6 atoms, selected from carbon, nitrogen, oxygen, and sulfur, where at most two ring atoms are non-carbon. The rings may comprise one or more double bonds in addition to the double bond of the above formula Examples of rings include phenyl, anthracyl, pyridinyl, and the like, wherein the atoms of the rings may be substituted with one or more groups such as, for example, alkyl hydroxy, nitro, oxo, and the like. X is linked to Ya through R⁷ or R⁸.

In Yb, R⁵ and R⁶ are as described above, and R¹⁰ and R¹¹ and R¹² are independently carboxyl or cyano or together form one or more rings, usually 2 to 3 rings, which, when more than one ring, are usually fused, wherein each ring may comprise 5 to 10 atoms, usually 5-6 atoms, selected from carbon, nitrogen, oxygen, and sulfur, where at most two ring atoms are non-carbon. The rings may comprise one or more double bonds in addition to the double bond of the above formula. Examples of rings include naphthyl, anthracyl, phenanthryl, and the like, wherein the atoms of the rings may be substituted with one or more moieties such as, for example, alkyl, hydroxy, nitro, oxo, and the like. X is linked to Y through R¹⁰ or R¹¹ or R¹².

When X is a linking group, it typically comprises a chain of 1-100, usually 1-70, preferably 1-50, and more preferably 2-30 atoms, each independently selected from carbon, oxygen, sulfur, nitrogen, and phosphorous. The number of heteroatoms in the chain is normally 0-20, usually 1-15, preferably 2-6. The atoms in the chain may be substituted with atoms other than hydrogen. As a general rule, the length of a particular linking group X can be selected arbitrarily to provide for convenience of synthesis.

The linking group may comprise functional groups such as, e.g., ether groups, carbonyl functionalities, both oxocarbonyl, e.g., aldehyde, and non-oxocarbonyl (including nitrogen and sulfur analogs) e.g., carboxy, amidine, amidate, thiocarboxy and thionocarboxy. Common functionalities resulting from synthetic formation of a covalent bond between the linking group and the moiety to which it is linked include ether, alkylamine, amidine, thioamide, urea, thiourea, guanidine, azo, thioether, carboxylate, sulfonate, and phosphate esters, amides, and thioesters. The linking group may comprise one or more polyoxyalkylene groups such as, for example, polyoxyethylene groups comprising from about 1 to about 100 oxyalkylene groups.

Y may be attached to the terminus of the linking group or, where more thin one Y is present, each Y may be attached at a point along the chain of the linking group, usually through the intermediacy of a functionality for binding Y to the chain.

Another specific embodiment of nucleosides that may be employed in the synthesis of polynucleotide e-tag probes is represented by the formula: 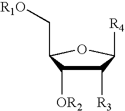
wherein R¹, R², R³ and R⁴, X and p are as described above, and
wherein Y is Ya′ or Yb′ as shown below:

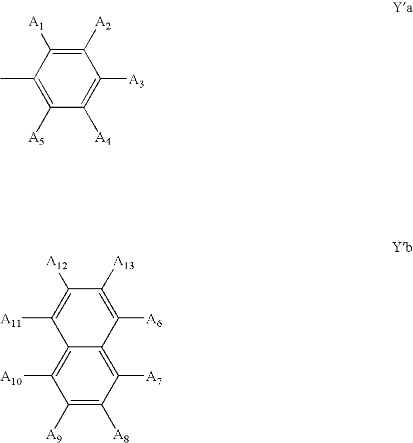

In Y′a, A¹ to A⁵ are independently selected from hydrogen, hydroxy, amino, substituted amino, carbonyl (as in aldehyde, keto and the like), carboxamide, and N-hydroxy amide, any of which may be bound to a protecting group such as, for example, acetyl, ethyl, isobutyryl, or t-butyryl, wherein at least two adjacent members of A¹ to A⁵, preferably at least two adjacent members of A² to A⁴, are not hydrogen. In Y′b, A⁶ and A⁷ are independently selected from hydroxy, amino, substituted amino, carbonyl (as in aldehyde, keto and the like), carboxamide, and N-hydroxy amide, any of which may be protecting group as above. One of A⁸ to A¹³ is linked to X, and the others are independently selected from the groups disclosed for A⁶ and A⁷ above.

Other specific embodiments of reagents that may be used to prepare e-tag probes are set forth in the following formulas, depicting a protected phosphoramidite reagent and derivatized solid support, respectively:

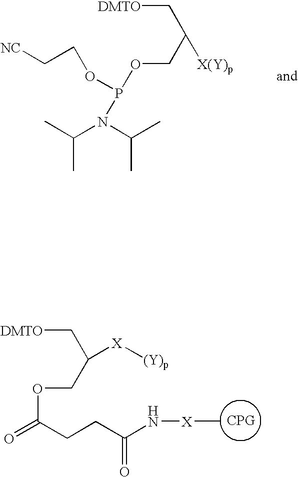

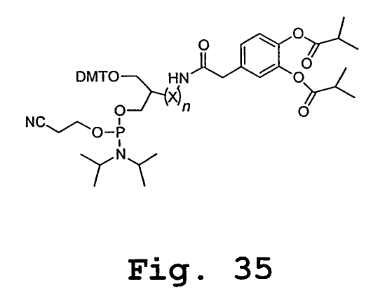

In these structures, X and Y are as described above and CPG represents a solid support, preferably controlled pore glass. Specific examples of compounds of the above formulas are set forth in FIGS. 35 and 49.

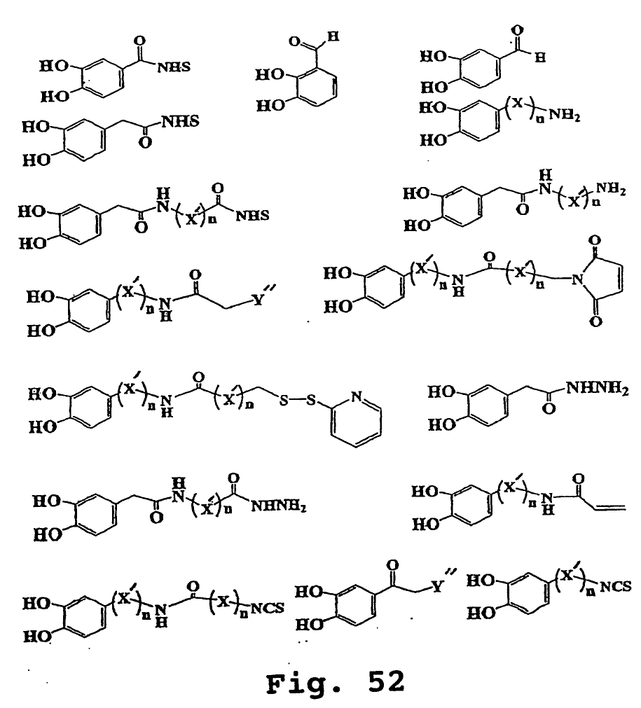

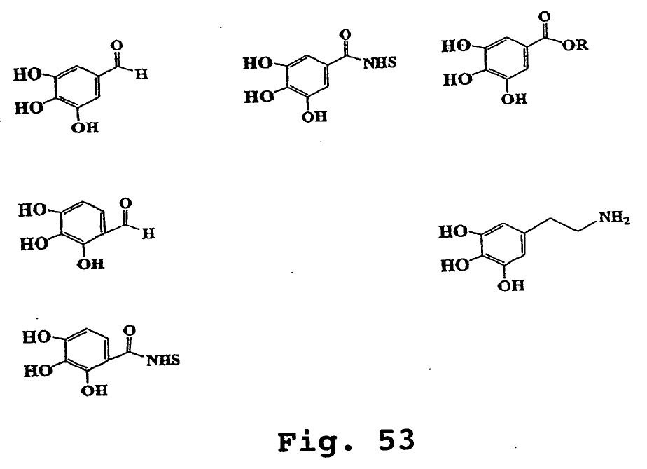

FIGS. 52 and 53 depict various catechol derivatives that comprise reactive functionalities for use in linking the compound with a nucleoside or oligonucleotide, by reaction with a corresponding functionality on the nucleoside or oligonucleotide. In the figures, X′ preferably represents alkylene, e.g. methylene, O, NH, S, or combinations thereof, n is 1 to 12, R is C₁-C₁₂ alkyl, and Y″ is Cl, Br, or I. The catechol ring may be further substituted with one or more moieties such as, for example, alkyl, nitro, hydroxyl, and so forth.

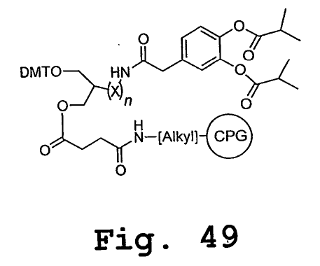

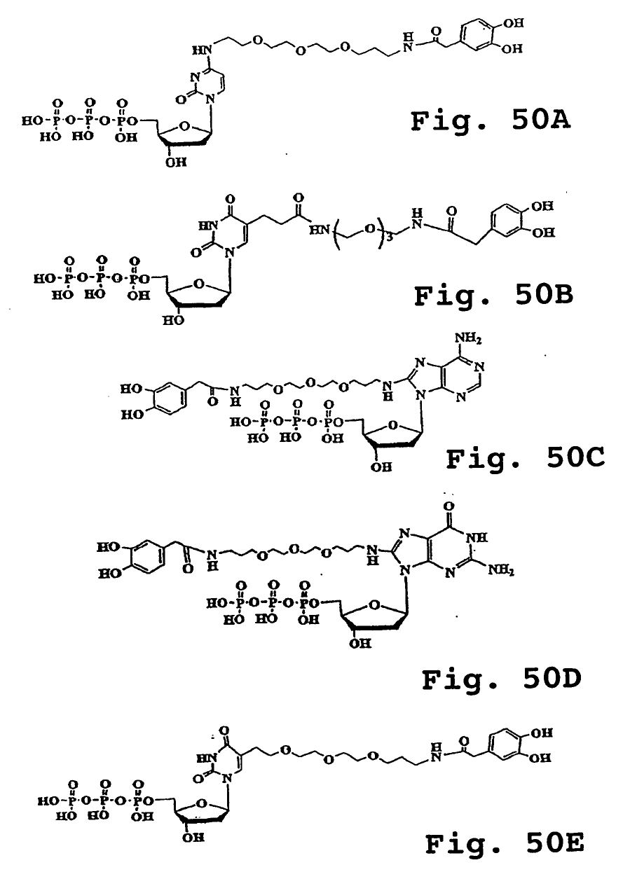

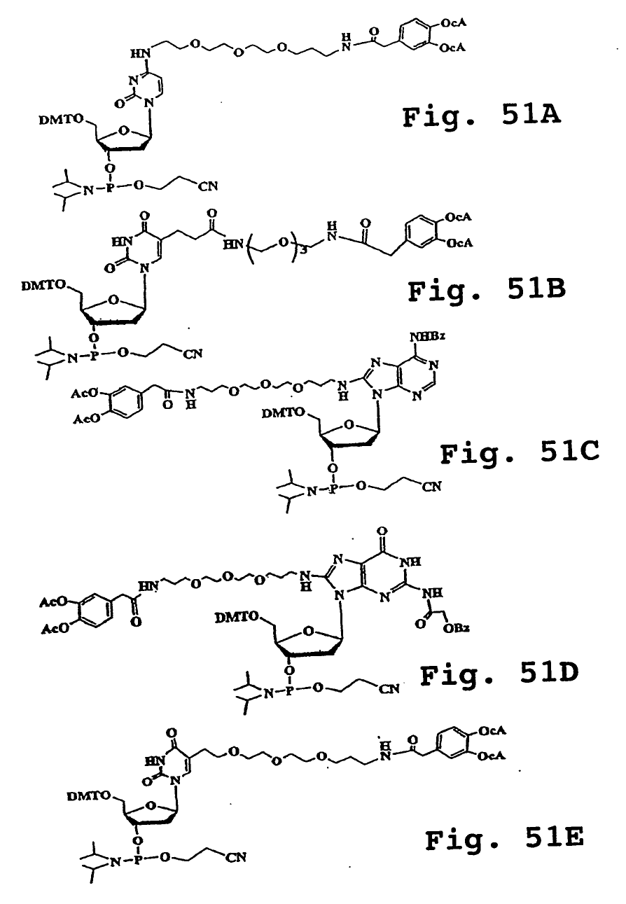

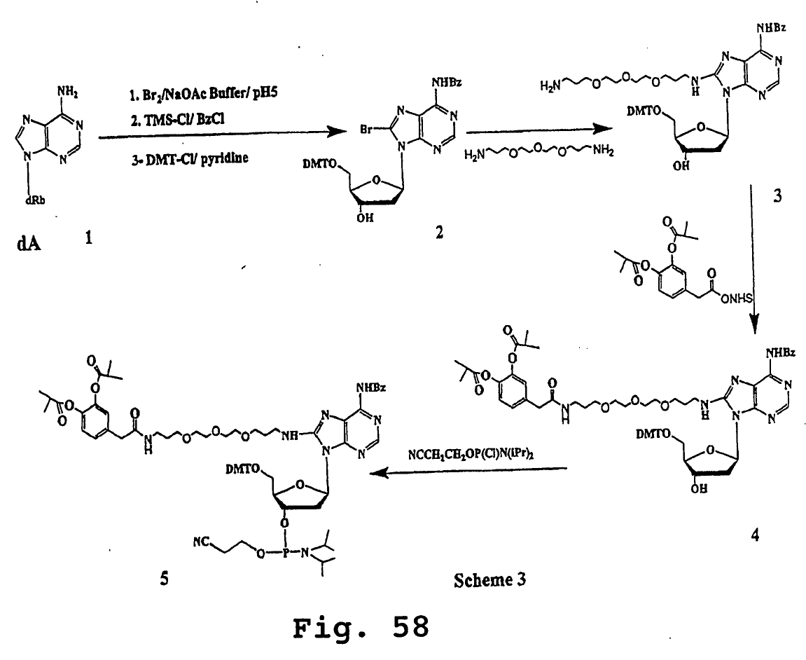

Certain specific embodiments of dNTP's of catechol-derivatized dC, dT, dA, dG and dU are shown in FIGS. 50A-50E. Furthermore, certain specific embodiments of phosphoramidites of catechol-derivatized dC, dT, dA, dG and dU are shown in FIGS. 51A-51E. The dNTP's and the phosphoramidites maybe synthesized according to procedures well known in the art, employing the synthetic techniques described herein for incorporation of the catechol moieties, as illustrated, for example, in FIGS. 58-62.

The derivatized monomers may be employed in the synthesis of polynucleotides and oligonucleotides comprising one or more catechol moieties, using oligonucleotide synthesis protocols known in the art, e.g. phosphate coupling, either direct phosphate coupling or via a phosphate precursor such as a phosphite. Such coupling thus includes the use of amidite (phosphoramidite), phosphodiester, phosphotriester, H-phosphonate, or phosphite halide intermediates. In a preferred approach, standard phosphoramidite chemistry is employed, preferably in the solid phase. In a typical procedure, a support bound monomer with a free 5′-hydroxyl group is reacted with a 5′-protected deoxyribonucleoside-3′-O—(N,N-diisopropylamino)phosphoramidite. The 5′ protecting group is removed after the coupling reaction, and the procedure is repeated with additional protected nucleotides until synthesis of the desired polynucleotide is complete. Oxidation can be effected with iodine to give phosphates or with sulfur to give phosphorothioates.

Incorporation of an organic moiety comprising a cis-diol into an oligonucleotide is also described in Cao et al., Chem. Pharm. Bull. (2000) 48(11) 1745.

The interactive functionality often requires protection during automated DNA synthesis. The protecting group is preferably acid-stable. Examples of protecting groups for diols, by way of illustration and not limitation, are carbonates, esters, and allelic-based protecting groups, which are removed during ammonolysis or via treatment with specialized reagents such as palladium in the case of allyloxy carbonyl groups. A dithiol may be protected as a disulfide or thioester. Other protecting groups for various functionalities are known in the art; see e.g. Kociensid, Protecting Groups, Georg Thieme Verlag, Stuttgart (1994).

In another exemplary embodiment, a boronic acid moiety may be incorporated as the interactive functionality at the penultimate nucleotide of an oligonucleotide reagent. Synthetic approaches include those described in, for example, U.S. Pat. Nos. 6,013,783, 5,876,938 and 6,031,117, which are incorporated herein by reference. One approach employs a modified nucleoside containing a boric or boronic acid moiety attached to a heterocyclic portion of the nucleoside. The attachment is made via a linking group that is typically from 7 to 30 carbon atoms in length and may comprise an aromatic ring. The linking group is of sufficient length that the boronic acid group can form a complex with a boronic acid complexing agent in an assay mixture.

III. Types of Assays

The assays described herein may involve nucleic acid detection, which includes sequence recognition, SNP detection and scoring, transcription analysis, allele determinations, HLA determinations, or other determination associated with variations in sequence. The use of the determination may be forensic, mRNA determinations, mutation determinations, allele determinations, MHC determinations, haplotype determinations, single nucleotide polymorphism determinations, etc. The methodology may include assays dependent on 5′-nuclease activity, as in the use of the polymerase chain reaction or in Invader™ technology, 3′-nuclease activity, restriction enzymes, or ribonuclease H. All of these methods involving catalytic cleavage of a phosphate linkage.

The assays may also be employed for detection of other types of specific binding member pairing. For example, there are a large number of specific binding pairs associated with receptors, such as antibodies, poly- and monoclonal, enzymes, surface membrane receptors, lectins, etc., and ligands for the receptors, which may be naturally occurring or synthetic molecules, protein or non-protein, such as drugs, hormones, enzymes, ligands, etc.

Probes comprising such target binding moieties are generally prepared with a specific cleavable linkage L, which may be cleavable chemically, photochemically, thermally, or enzymatically, as described above, while oligonucleotide based probes are commonly cleaved at a phosphate internucleotide linkage. Non-oligonucleotide probes are preferably designed such that the interactive functionality, as discussed above, is present at a position on the probe adjacent the cleavable linkage, but on the other side of the linkage from the releasable reporter group. As discussed above, in an oligonucleotide probe, the reporter group is typically placed on the terminal nucleotide, with the interactive functionality on the penultimate nucleotide.

Tables 3 and 4 provide a non-exclusive list of the various assays in which the subject compositions and methods may be used.

TABLE 3
Binding and Multiplexed Assays

| | | | e-tag Reporter |
|---|---|---|---|
| Format | Recognition Event | Amplification Mode | Release |
| Multiplexed | Solution hybridization | PCR, Invader | 5′ nuclease |
| assays | followed by enzyme | | 3′ nuclease |
| Sequence | recognition | | Restriction |
| recognition e.g. | | | enzyme |
| multiplexed gene | | | Ribonuclease H |
| expression, SNPs | Solution hybridization | Amplification due to | Singlet oxygen |
| | followed by channeling | turnover of e-tag probe | (¹O₂) |
| | | or release of multiple | Hydrogen |
| | | e-tag reporters (10 to | peroxide (H₂O₂) |
| | | 100,000) per binding | Light, |
| | | event | energy transfer |
| Patches in | Target captured on solid | Amplification from | Light, enzyme, |
| microfluidic | surface; e-tag probe | release of multiple e-tag | ¹O₂, H₂O₂, |
| channels - | mixture hybridized to | reporters (10 to | fluoride, |
| integrated assay | target; unbound probes | 100,000) per probe | reducing agents |
| and separation | removed; e-tag reporter | | |
| device | released, separated and | | |
| | identified | | |

TABLE 4
Immunoassays

| | | | e-tag Reporter |
|---|---|---|---|
| Format | Recognition Event | Amplification Mode | Release |
| Proteomics | Sandwich assays | Amplification from | Singlet oxygen |
| Multiplexed | Competition assays | release of multiple | (¹O₂) |
| Immunoassays | Antibody-1 conjugated | e-tag reporters (10 to | |
| | with sensitizer; antibody-2 | 100,000) per binding | |
| | conjugated with singlet | event | |
| | oxygen-cleavable e-tag | | |
| | groups | | |
| | Sandwich assays | | Hydrogen peroxide |
| | Competition assays | | (H₂O₂) |
| | Antibody-1 conjugated | | |
| | with glucose oxidase; | | |
| | antibody-2 conjugated | | |
| | with hydrogen peroxide- | | |
| | cleavable e-tag groups | | |
| Patches in | Sandwich assays | | Light; enzymes, |
| microfluidic | Competition assays | | singlet oxygen, |
| channels; | Antibody-1 attached to | | hydrogen peroxide, |
| integrated assay | solid surface; antibody-2 | | fluoride, reducing |
| and separation | conjugated with cleavable | | agents |
| device | e-tag groups | | |

The assays may be performed in a competitive mode or a sandwich mode. In the competitive mode, the target competes with a labeled binding member for the reciprocal member. The reciprocal member is bound to the support, either during the complex formation or after, e.g. where an antibody is a specific binding member and anti-immunoglobulin is the reciprocal binding member and is bound to the support. In this mode, the binding sites of the reciprocal binding member become at least partially filled by the target, reducing the number of available binding sites for the labeled reciprocal binding member. Thus, the number of labeled binding members that bind to the reciprocal binding member will be in direct proportion to the number of target molecules present. In the sandwich mode, the target is able to bind at the same time to different binding members; a first support bound member and a second member that binds at a site of the target molecule different from the site at which the support bound member binds. The resulting complex has three components, where the target serves to link the labeled binding member to the support

The subject libraries of e-tag reagents may be used to analyze the effect of an agent on a plurality of different compounds. For example, a plurality of substrates labeled with an e-tag moiety may be prepared, where the enzyme catalyzes a reaction resulting in a change in mobility between the product and the starting material. These assays can find use in determining affinity groups or preferred substrates for hydrolases, oxidoreductases, lyases, etc. For example, with kinases and phosphatases, a charged group is added or removed so as to change the mobility of the product. By preparing a plurality of alcohols or phosphate esters, a determination may be made concerning which of the compounds serves as a substrate. By labeling the substrates with e-tag moieties, the shift from the substrate to the product can be observed as evidence of the activity of a candidate substrate with the enzyme. By preparing compounds as suicide inhibitors, the enzymes may be sequestered and the e-tag reporters released to define those compounds that may serve as suicide inhibitors and, therefore, preferentially bind to the active site of the enzyme.

The subject methods may be used for screening for the activity of one or more candidate compounds, particularly drugs, for their activity against a battery of enzymes. In this situation, active substrates for each of the enzymes to be evaluated may be used, where each of the substrates has its own e-tag probe. For those enzymes for which the drug is an inhibitor, the amount of product is diminished in relation to the amount of product in the absence of the candidate compound. In each case the product has a different mobility from the substrate, so that the substrates and products can be readily distinguished by electrophoresis. By appropriate choice of substrates and detectable labels, electropherograms may be obtained showing the effect of the candidate compound on the activity of the different enzymes.

Assays employing oligonucleotide probes generally involve the formation of a cleavage structure; that is, a complex of target nucleic acid and specifically bound electrophoretic probe that is subject to enzymatic cleavage, to release an etag reporter group from the probe. Particular examples of such assays are described in more detail below.

A. Primer Extension Reaction in Nucleic Acid Analyses

The extension reaction is performed by bringing together the necessary combination of reagents, then subjecting the mixture to conditions for carrying out the desired primer extension. Such conditions depend on the nature of the extension, e.g. PCR, single primer amplification, LCR, NASBA, 3SR and so forth, where the enzyme that is used for the extension has 5′-3′ nuclease activity. The extension reaction may be carried out as to both strands or as to only a single strand. Where pairs of primer and SNP detection sequence are used for both strands, conveniently, the e-tag moiety is the same but the bases are different. In this situation, a cleavable linkage to the base is employed, so that for the same SNP, the same e-tag reporter is obtained. Alternatively, if the number of SNPs to be determined is not too high, different e-tag moieties can be used for each of the strands. Usually, the reaction is carried out by using amplifying conditions, so as to provide an amplified signal for each SNP. Amplification conditions normally employ thermal cycling, where after the primer extension and release of electrophoretic tag reporters associated with SNPs which are present, the mixture is heated to denature the double-stranded DNA, cooled, where the primer and SNP detection sequence can rehybridize and the extension be repeated.

Reagents for conducting the primer extension are substantially the same reaction materials for carrying out an amplification, such as an amplification indicated above. The nature and amounts of these reagents are dependent on the type of amplification conducted. In addition to oligonucleotide primers, the reagents also comprise nucleoside triphosphates and a nucleotide polymerase having 5′-3′ nuclease activity.

The nucleoside triphosphates employed as reagents in an amplification reaction include deoxyribonucleoside triphosphates such as the four common deoxyribonucleoside triphosphates dATP, dCTP, dGTP and dTTP. The term “nucleoside triphosphates” also includes derivatives and analogs thereof, which are exemplified by those derivatives that are recognized and polymerized in a similar manner to the underivatized nucleoside triphosphates.

The nucleotide polymerase employed is a catalyst, usually an enzyme, for forming an extension of an oligonucleotide primer along a polynucleotide such as a DNA template, where the extension is complementary thereto. The nucleotide polymerase is a template dependent polynucleotide polymerase and utilizes nucleoside triphosphates as building blocks for extending the 3′-end of a polynucleotide to provide a sequence complementary with the polynucleotide template. Usually, the catalysts are enzymes, such as DNA polymerases, for example, prokaryotic DNA polymerase (I, II, or III), T4 DNA polymerase, T7 DNA polymerase, Vent DNA polymerase, Pfu DNA polymerase, Taq DNA polymerase, and the like. Polymerase enzymes may be derived from any source, such as eukaryotic or prokaryotic cells, bacteria such as E. coli, plants, animals, virus, thermophilic bacteria, genetically modified enzymes, and so forth.

The conditions for the various amplification procedures are well known to those skilled in the art. In a number of amplification procedures, thermal cycling conditions as discussed above are employed to amplify the polynucleotides. The combination of reagents is subjected to conditions under which the oligonucleotide primer hybridizes to the priming sequence of, and is extended along, the corresponding polynucleotide. The exact temperatures can be varied depending on the salt concentration, pH, solvents used, length of and composition of the target polynucleotide sequence and the oligonucleotide primers.

Thermal cycling conditions are employed for conducting an amplification involving temperature or thermal cycling and primer extension such as in PCR or single primer amplification, and the like. The pH and the temperature are selected so as to cause, either simultaneously or sequentially, dissociation of any internally hybridized sequences, hybridization or annealing of the oligonucleotide primer and SNP detection sequence with the target polynucleotide sequence, extension of the primer, release of the e-tag reporter from SNP detection sequence bound to the target polynucleotide sequence, and dissociation of the extended primer. This usually involves cycling the reaction medium between two or more temperatures. In conducting such a method, the medium is cycled between two to three temperatures. Temperatures for thermal cycling generally range from 50-100° C., more usually from 60-95° C. Relatively low temperatures of 30-65° C. can be employed for the extension steps, while denaturation and hybridization are generally carried out at about 50-105° C. The reaction medium is initially held at about 20-45° C., preferably about 25-35° C. Relatively low temperatures of from about 50-80° C. preferably 50-60° C., are employed for the hybridization or annealing steps, while denaturation is carried out at about 80-100° C., preferably 90-95° C., and extension is carried out at about 70-80° C., usually about 72-74° C. The duration of each cycle is usually about 1-120 seconds, preferably about 5-60 seconds, for the denaturation steps, and usually about 1-15 seconds, preferably about 1-5 seconds, for the extension steps. It is to be understood that the actual temperature and duration of the cycles employed are dependent on the particular amplification conducted and are well within the knowledge, of those skilled in the art.

Generally, an aqueous medium is employed. Other polar co-solvents may also be employed, usually oxygenated organic solvents of from 1-6, more usually from 1-4, carbon atoms, including alcohols, ethers, formamide and the like. Usually, these co-solvents, if used, are present in less than about 70 weight percent, more usually in less than about 30 weight percent.

The pH for the medium is usually in the range of about 4.5 to 9.5, more usually in the range of about 5.5 to 8.5, and preferably in the range of about 6 to 8. Various buffers may be used to achieve the desired pH and maintain the pH during the determination. Illustrative buffers include borate, phosphate, carbonate, Tris, barbital and the like. The particular buffer employed is not critical to this invention but in individual methods one buffer may be preferred over another. The medium may also contain materials required for enzyme activity such as a divalent metal ion (usually magnesium).

Various ancillary materials will frequently be employed in the methods in accordance with the present invention. For example, in addition to buffers and salts, the medium may also comprise stabilizers for the medium and the reaction components. Frequently, the medium may also include proteins such as albumins, quaternary ammonium salts, polycations such as spermine, surfactants, particularly non-ionic surfactants, binding enhancers, e.g., polyalkylene glycols, or the like.

The reaction is conducted for a time sufficient to produce the desired number of copies of each of the polynucleotides suspected of being present as discussed below. Generally, the time period for conducting the entire method will be from about 10 to 200 minutes. As mentioned above, it is usually desirable to minimize the time period.

The concentration of the nucleotide polymerase is usually determined empirically. Preferably, a concentration is used that is sufficient such that the amplification is robust. The primary limiting factor generally is the cost of the reagent. Such enzymes include Pfu DNA polymerase (native and recombinant) from Stratagene, La Jolla, Calif., U1Tma DNA polymerase from Perkin Elmer, Foster City, Calif., rBst DNA polymerase from Epicentre Technologies, Madison, Wis., Vent DNA polymerase from New England Biolabs, Beverly, Mass., Tli DNA polymerase from Promega Corp., Madison, Wis., and Pwo DNA polymerase from Boehringer Mannheim, Indianapolis, Ind., and the like.

The initial concentration of each of the polynucleotides containing the respective target-binding moiety for the target SNPs can be as low as about 50 pg/μL in a sample. After amplification the concentration of each polynucleotide should be at least about 10 pM, generally in the range of about 10 pM to about 10 nM, usually from about 10 to about 10¹⁰, more usually from about 10³ to about 10⁸ molecules in a sample, preferably at least about 10⁻²¹ M in the sample and may be about 10⁻¹⁰ to about 10⁻¹⁹ M, more usually about 10⁻¹⁴ to about 10⁻¹⁹M. In general, the reagents for the reaction are provided in amounts to achieve extension of the oligonucleotide primers.

The concentration of the oligonucleotide primer(s) will be about 1 to about 20 μM and is usually about 1 to about 10 μM, preferably, about 1 to about 4 μM, for a sample size that is about 10 fM. Preferably, the concentration of the oligonucleotide primer(s) is substantially in excess over, preferably at least about 10⁷ to about 10¹⁰ times greater than, more preferably, at least about 10⁹ times greater than, the concentration of the corresponding target polynucleotides.

The amount of the oligonucleotide probes will be 10 to about 500 nM and is usually about 50 to about 200 nM for a sample size that is about 10 fM. Preferably, the concentration of the oligonucleotide probes is substantially in excess over, preferably at least about 10⁷ times greater than, more preferably, at least about 10⁸ times greater than, the concentration of each of the target polynucleotides.

The concentration of the nucleoside triphosphates in the medium can vary widely; preferably, these reagents are present in an excess amount. The nucleoside triphosphates are usually present in about 10 μM to about 1 mM, preferably, about 20 to about 400 μM.

The order of combining of the various reagents to form the combination may vary. Usually, the sample containing the polynucleotides is combined with a pre-prepared combination of nucleoside triphosphates and nucleotide polymerase. The oligonucleotide primers and the SNP detection sequences may be included in the prepared combination or may be added subsequently. However, simultaneous addition of all of the above, as well as other step-wise or sequential orders of addition, may be employed provided that all of the reagents described above are combined prior to the start of the reactions. The oligonucleotide pairs may be added to the combination of the reagents at or prior to initiation of the primer extension reaction and may be replenished from time-to-time during the primer extension reaction.

For quantitation, one may choose to use controls, which provide a signal in relation to the amount of the target that is present or is introduced. Where one is dealing with a mixture of nucleic acid molecules, as in the case of mRNA in a lysate, one may use the known amounts of one or more different mRNA's in the particular cell types as the standards. Desirably, one would have at least two controls, preferably at least 3 controls, where the variation in number between any two controls is at least about 10², and the total range is at least about 10³, usually at least about 10⁴. However, determining the consistent ratio of mynas occurring naturally may result in a large margin of error, so that one would usually rely on synthetic targets as the control. Where a control system is added for quantitation, as compared to relying on the presence of a known amount of a plurality of endogenous nucleic acids, the control system will comprise at least two control sequences, usually at least 3 control sequences and generally not more than about 6 control sequences, where the upper limit is primarily one of convenience and economy, since additional control sequences will usually not add significant additional precision. The control sequences will usually be at least about 50 nucleotides, more usually at least about 100 nucleotides. The control sequences will have a common primer sequence and different control detection sequences, which are intended to parallel the primer sequence and SNP detection sequence in size, spacing and response to the primer extension conditions. In carrying out the primer extension reaction with sample nucleic acid, one would then add different number of molecules of the different control sequences, so that one could graph the result to give a signal/number relationship. This graph could then be used to relate signals observed with target molecules to the number of molecules present.

As exemplary of the subject invention, four target polynucleotides T1, T2, T3 and T4 are employed. Oligonucleotide primers PR1, PR2, PR3 and PR4 are employed, each respectively capable of hybridizing to a sequence in the respective target polynucleotides. Also employed are four oligonucleotide SNP detection sequences, (electrophoretic probes) PB1, PB2, PB3 and PB4. Each of the SNP detection sequences comprises a fluorescent label F1, F2, F3 and F4, respectively. In this example, there is a mismatch between PB2 and T2, which comprises a single nucleotide polymorphism. The reaction medium comprising the above reagents and nucleoside triphosphates and a template dependent polynucleotide polymerase having 5′ to 3′ exonuclease activity is treated under amplification conditions. Primers PR1, PR2, PR3 and PR4 hybridize. to their respective target polynucleotides and are extended to yield extended primers EPR1, EPR2, EPR3 and EPR4. SNP detection sequences PB1, PB3. and PB4, which hybridize with their respective target polynucleotides, are acted upon by the exonuclease to cleave a single nucleotide bearing the respective fluorescent label. PB2, which does not bind to the target polynucleotide, is not cleaved. Cleaved fragments F1, F3 and F4 are injected into a separation channel in a chip for conducting electroseparation. The labels are identified by their specific mobility and fluorescence upon irradiation. The separated labels are related to the presence and amount of the respective target polynucleotide.

The selection of the SNP detection or other target binding sequence will affect the stringency employed during the primer extension, particularly at the stage of hybridization. Since in a substantial number of samples, the DNA will be heterozygous for SNPs, rather than homozygous, one does not wish to have false positives, where the SNP detection sequence may bond to the sequence comprising the prevalent nucleotide, as well as the sequence comprising the SNP. Where the DNA sample is homozygous for the prevalent sequence, it is also important that the target binding sequence does not bind to give a false positive. Therefore, the difference in Tₘ between the target containing sequence and the wild-type sequence will usually be at least about 3° C., more usually at least about 5° C., under the conditions of the primer extension.

In one exemplary protocol, the tagged SNP detection sequence will be chosen to bind to the target sequence comprising the SNP. The length of the SNP detector sequence is in part related to the length and binding affinity of the primer. The two sequences act together to ensure that the pair of reagents bind to the proper target sequence. The greater the fidelity of binding of one member of the pair, the less fidelity that is required for the other member of the pair. Since the observed signal will be dependent upon both members of the pair being present, each member serves as a check on the other member for production of the signal. However, since except for the cost, it is relatively easy to make reasonably long oligonucleotides, usually both members of the pair will uniquely hybridize to their respective target sequences. Therefore, the length of the SNP detector sequence will come within the parameters indicated for the primer, but the total number of bases for the two pair members will usually be at least 36, more usually at least about 40.

Depending on the protocol, an e-tag reporter will be separated from a portion or substantially all of the detection sequence, usually retaining not more than about 3 nucleotides, more usually not more than about 2 nucleotides and preferably from 0 to 1 nucleotide. By having a cleavable linker between the e-tag moiety and the detection sequence, the e-tag reporter may be freed of all the nucleotides. By having a nuclease-resistant penultimate link, a single nucleotide may be bonded to the e-tag reporter.

Each SNP detection sequence will have at least one nucleotide modified with an electrophoretic tag, which is fluorescent or can be subsequently made fluorescent, or can be detected electrochemically or by other convenient detection methodologies. Usually, the modified nucleotide will be at the 5′-end of the sequence, but the modified nucleotide may be anywhere in the sequence, particularly where there is a single nuclease susceptible linkage in the detection sequence. Since the determination is based on at least partial degradation of the SNP detector sequence, having the modified nucleotide at the end ensures that if degradation occurs, the electrophoretic tag will be released.

Since nucleases may cleave at other than the terminal phosphate link, it is desirable to reduce such cleavage by the use of nuclease-resistant linkages, e.g. a thiophosphate, phosphinate, phosphoramidate, or amide, at other than the terminal phosphate link However, such modification is less critical in view of the present invention, in which an interactive functionality is preferably attached at the penultimate nucleotide of the electrophoretic probe, with the etag reporter group at the terminal nucleotide, which is specifically cleaved by the nuclease in accordance with the assay.

The pairs of reagents are DNA sequences that are related to a SNP site. The primer binds to the target DNA upstream from the SNP site in the direction of extension. The labeled detector sequence binds downstream from the primer in the direction of extension and binds to a sequence, which includes the SNP. The primer sequence will usually be at least about 12 bases long, more usually at least 18 bases long and usually fewer than 100 bases, and more usually fewer than 60 bases. The primer will be chosen to bind substantially uniquely to a target sequence under the conditions of primer extension, so that the sequence will normally be one that is conserved or the primer is long enough to bind in the presence of a few mismatches, usually fewer than about 10 number %, mismatches. By knowing the sequence, which is upstream from the SNP of interest, one may select a sequence, which has a high G-C ratio, so as to have a high binding affinity for the target sequence. In addition, the primer should bind reasonably close to the SNP, usually not more than about 200 bases away, more usually not more than about 100 bases away, and preferably within about 50 bases. Since the farther away the primer is from the SNP, the greater amount of dNTPs that will be expended, there will usually be no advantage in having a significant distance between the primer and the SNP detection sequence. Generally, the primer will be at least about 5 bases away from the SNP.

The complementary base to the SNP may be anywhere in the detector sequence, desirably at other than the terminal nucleoside to enhance the fidelity of binding. The SNP detector sequence will be designed to include adjacent nucleotides, which provide the desired affinity for the hybridization conditions. The SNP detection sequence may be synthesized by any convenient means, such as described in Matthews et al., Anal. Biochem. (1988) 169:1-25; Keller et al., “DNA Probes,” ₂nd edition (1993) Stockton Press, New York, N.Y.; and Wetmur, Critical Reviews in Biochemistry and Molecular Biology (1991) 26:227-259.

A plurality of SNPs can be simultaneously determined by combining target DNA with a plurality of reagent pairs (primers and electrophoretic probes) under conditions of primer extension. Each pair of reagents includes a primer which binds to target DNA and a SNP detection sequence (probe) which binds to the site of the SNP and includes an e-tag reporter group, usually at its 5′-end and the base complementary to the SNP. The conditions of primer extension employ a polymerase having 5′-3′ exonuclease activity, dNTP's and auxiliary reagents to permit efficient primer extension. The primer extension is performed, whereby detector sequences bound to the target DNA are degraded with release of the e-tag reporter. By having each probe, and thus each SNP, associated with its own e-tag reporter, one can determine the SNPs which are present in the target DNA. Typically, the total number of different reagent pairs or different target sequences in a single determination will be under 200, more usually under 100, and in many cases will not exceed 50.

B. The Invader™ Reaction in Nucleic Acid Analyses

In one SNP determination protocol, the primer includes the complementary base of the SNP. This protocol is referred to as Invader™ technology, and is described in U.S. Pat. No. 6,001,567. The protocol involves providing: (a) (i) a cleavage means, which is normally an enzyme, referred to as a cleavase, that recognizes a triplex consisting of the target sequence, a primer which binds to the target sequence and terminates at the SNP position and a labeled probe that binds immediately adjacent to the primer and is displaced from the target at the SNP position, when a SNP is present. The cleavase clips the labeled probe at the site of displacement, releasing the label, (ii) a source of target nucleic acid, the target nucleic acid having a first region, a second region and a third region, wherein the first region is downstream from the second region and the second region is contiguous to and downstream from the third region, and (iii) first and second oligonucleotides having 3′ and 5′ portions, wherein the 3′ portion of the first oligonucleotide contains a sequence complementary to the third region of the target nucleic acid and the 5′ portion of the first oligonucleotide and the 3′ portion of the second oligonucleotide each contain sequences usually fully complementary to the second region of the target nucleic acid, and the 5′ portion of the second oligonucleotide contains sequence complementary to the first region of said target nucleic acid; (b) mixing, in any order, the cleavage means, the target nucleic acid, and the first and second oligonucleotides under hybridization conditions that at least the 3′ portion of the first oligonucleotide is annealed to the target nucleic acid and at least the 5′ portion of the second oligonucleotide is annealed to any target nucleic acid to from a cleavage structure, where the combined melting temperature of the complementary regions within the 5′ and 3′ portions of the first oligonucleotide when annealed to the target nucleic acid is greater than the melting temperature of the 3′ portion of the first oligonucleotide and cleavage of the cleavage structure occurs to generate labeled products; and (c) detecting the labeled cleavage products.

Thus, in an Invader assay, attachment of an e-tag moiety to the 5′ end of the detector sequence results in the formation of an e-tag-labeled nucleotide when the target sequence is present. The e-tag-labeled nucleotide is separated and detected. By having a different e-tag reporter for each nucleic acid sequence of interest, each having a different electrophoretic mobility, one can readily determine the SNPs or measure multiple sequences, which are present in a sample. The e-tag groups may require further treatment, depending on the total number of SNPs or target sequences being detected.

III. Exemplary Protocols for Use of etag Reagents

In carrying out the assays, the components are combined, usually with the target composition added first and then the labeled members in the competitive mode and in any order in the sandwich mode. Usually, the labeled member in the competitive mode will be equal to at least about 50% of the highest number of target molecules anticipated, preferably at least equal and may be in about 2 to about 10 fold excess or greater. The particular ratio of target molecules to labeled molecules will depend on the binding affinities, the length of time the mixture is incubated, the off rates for the target molecule with its reciprocal binding member, the size of the sample and the like. In the case of the sandwich assays, one will have at least an equal amount of the labeled binding member to the highest expected amount of the target molecules, usually at least about 1.5 fold excess, more usually at least about 2 fold excess and may have about 10 fold excess or more. The components are combined under binding conditions, usually in an aqueous medium, generally at a pH in the range of about 5—about 10, with buffer at a concentration in the range of about 10 to about 200 mM. These conditions are conventional, where conventional buffers may be used, such as phosphate, carbonate, HEPES, MOPS, Tris, borate, etc., as well as other conventional additives, such as salts, stabilizers, organic solvents, etc.

Generally, the concentrations of the various agents involved with the signal producing system will vary with the concentration range of the individual analytes in the samples to be analyzed, generally being in the range of about 10 nM to about 10 mM. Buffers will ordinarily be employed at a concentration in the range of about 10 to about 200 mM. The concentration of each analyte will generally be in the range of about 1 pM to about 100 μM, more usually in the range of about 100 pM to about 10 μM. In specific situations the concentrations may be higher or lower, depending on the nature of the analyte, the affinity of the reciprocal binding members, the efficiency of release of the e-tag reporters, the sensitivity with which the e-tag reporters are detected, and the number of analytes, as well as other considerations.

Sample preparation may include, for example, lysis of cells, nucleic acid separation from proteins and lipids and vice versa, and enrichment of different fractions. For nucleic acid related determinations, the source of the DNA may be any organism, prokaryotic and eukaryotic cells, tissue, environmental samples, etc. The DNA or RNA may be isolated by conventional means, RNA may be reverse transcribed, DNA may be amplified, as with PCR, primers may be used with capture ligands for use in subsequent processing, the DNA may be fragmented using restriction enzymes, specific sequences may be concentrated or removed using homologous sequences bound to a support, or the like. Proteins may be isolated using precipitation, extraction, and chromatography. The proteins may be present as individual proteins or combined in various aggregations, such as organelles, cells, viruses, etc. Once the target components have been preliminarily treated, the sample may then be combined with the e-tag reporter targeted binding proteins.

For a nucleic acid sample, after processing, the mixture of e-tag probes for the target sequences is combined with the sample under hybridization conditions, in conjunction with other reagents, as necessary. Following incubation and binding of probe to target sequence, enzyme (or other cleavage agent) is added to cleave reporter groups in target-bound probes.

The cleavage of the nucleic acid bound to the template results in a change in the melting temperature of the e-tag residue with release of the e-tag reporter. By appropriate choice of the primer and/or protocol, one can retain the primer bound to the template and the e-tag-containing sequence can be cleaved and released from the template to be replaced by further e-tag-containing probe.

The resulting cleavage mixture is then preferably treated with a capture agent on a solid support, as described above, to sequester species containing the interactive functionality from the cleaved reporter groups. Where particles or beads are employed, these may be separated by filtration, centrifugation, magnetic separation, etc.

Where detectable labels are not present on the e-tag moieties, the moieties may be reacted with detectable labels. In some instances the detectable label may be part of the reagent cleaving the cleavable bond, e.g. a disulfide with a thiol. Where there is a plurality of different functionalities on different binding members for reaction with the label, the different labels will have functionalities that react with one of the functionalities. The different labels may be added together or individually in a sequential manner. For example, where the functionalities involve thiols, carboxyl groups, aldehydes and olefins, the labels could have activated olefins, alcohols, amines and thiol groups, respectively. By having removable protective groups for one or more of the functionalities, the protective groups may be removed stepwise and the labels added stepwise. In this way cross-reactivity may be avoided. Whether one has the detectable label present initially or one adds the detectable label is not critical to this invention and will frequently be governed by the nature of the target composition, the nature of the labeled binding members, and the nature of the detectable labels. For the most part, it will be a matter of convenience as to the particular method one chooses for providing the detectable label on the e-tag reporter.

Once a solution of e-tag reporters is prepared and free of any interfering components, the solution may be analyzed electrophoretically. The analysis may employ capillary electrophoresis devices, microfluidic devices or other devices that can separate a plurality of compounds electrophoretically, providing resolved bands of the individual e-tag reporters.

IV. Analysis of Assay Reaction Products

The separation of the e-tag reporters by electrophoresis can be performed in conventional ways. See, for example, U.S. Pat. Nos. 5,750,015, 5,866,345, 5,935,401, 6,103,199, and 6,110,343, and WO98/5269, and references cited therein. Also, the sample can be prepared for mass spectrometry in conventional ways. See, for example, U.S. Pat. Nos. 5,965,363, 6,043,031, 6,057,543, and 6,111,251.

After completion of the primer extension reaction, either by monitoring the change in fluorescence as described above or taking aliquots and assaying for total free e-tag reporters, the mixture may now be analyzed. Depending on current instrument, from one to four different fluorescers activated by the same light source and emitting at different detectable labels may be used. With improvements, five or more different fluorescers will be available, where an additional light source may be required. Electrochemical detection is described in U.S. Pat. No. 6,045,676.

The presence of each of the cleaved e-tag reporters is determined by the label. The separation of the mixture of labeled e-tag reporters is typically carried out by electroseparation, which involves the separation of components in a liquid by application of an electric field, preferably, by electrokinesis (electrokinetic flow) electrophoretic flow, or electroosmotic flow, or combinations thereof, with the separation of the e-tag reporter mixture into individual fractions or bands. Electroseparation involves the migration and separation of molecules in an electric field based on differences in mobility. Various forms of electroseparation include, by way of example and not limitation, free zone electrophoresis, gel electrophoresis, isoelectric focusing and isotachophoresis. Capillary electroseparation involves electroseparation, preferably by electrokinetic flow, including electrophoretic, dielectrophoretic and/or electroosmotic flow, conducted in a tube or channel of about 1-200 micrometer, usually, about 10-100 micrometers cross-sectional dimensions. The capillary may be a long independent capillary tube or a channel in a wafer or film comprised of silicon, quartz, glass or plastic.

In capillary electroseparation, an aliquot of the reaction mixture containing the e-tag products is subjected to electroseparation by introducing the aliquot into an electroseparation channel that may be part of; or linked to, a capillary device in which the amplification and other reactions are performed. An electric potential is then applied to the electrically conductive medium contained within the channel to effectuate migration of the components within the combination. Generally, the electric potential applied is sufficient to achieve electroseparation of the desired components according to practices well known in the art. One skilled in the art will be capable of determining the suitable electric potentials for a given set of reagents used in the present invention and/or the nature of the cleaved labels, the nature of the reaction medium and so forth. The parameters for the electroseparation including those for the medium and the electric potential are usually optimized to achieve maximum separation of the desired components. This may be achieved empirically and is well within the purview of the skilled artisan.

For a homogeneous assay, the sample, e-tag-labeled probe mixture, and ancillary reagents are combined in a reaction mixture supporting the cleavage of the linking region. The mixture may be processed to separate the e-tag reporters from the other components of the mixture. The mixture, with or without e-tag reporter enrichment, may then be transferred to an electrophoresis device, usually a microfluidic or capillary electrophoresis device and the medium modified as required for the electrophoretic separation. Where it is desired to remove from the separation channel intact e-tag reporter molecules, an interactive functionality may be employed as described above.

Capillary devices are known for carrying out amplification reactions such as PCR. See, for example, Analytical Chemistry (1996) 68:4081-4086. Devices are also known that provide functional integration of PCR amplification and capillary electrophoresis in a microfabricated DNA analysis device. One such device is described by Woolley et al., in Anal. Chem. (1996) 68:4081-4086. The device provides a microfabricated silicon PCR reactor and glass capillary electrophoresis chips. EI the device a PCR chamber and a capillary electrophoresis chip are directly linked through a photolithographically fabricated channel filled with a sieving matrix such as hydroxyethylcellulose. Electrophoretic injection directly from the PCR chamber through the cross injection channel is used as an “electrophoretic valve” to couple the PCR and capillary electrophoresis devices on a chip.

The capillary electrophoresis chip contains a sufficient number of main or secondary electrophoretic channels to receive the desired number of aliquots from the PCR reaction medium or the solutions containing the cleaved labels, etc., at the intervals chosen.

For capillary electrophoresis one or more detection zones may be employed to detect the separated cleaved labels. It is, of course, within the purview of the present invention to utilize several detection zones depending on the nature of the amplification process, the number of cycles for which a measurement is to be made and so forth. There may be any number of detection zones associated with a single channel or with multiple channels. Suitable detectors for use in the detection zones include, by way of example, photomultiplier tubes, photodiodes, photodiode arrays, avalanche photodiodes, linear and array charge coupled device (CCD) chips, CCD camera modules, spectrofluorometers, and the like. Excitation sources include, for example, filtered lamps, LED's, laser diodes, gas, liquid and solid-state lasers, and so forth. The detection may be laser scanned excitation, CCD camera detection, coaxial fiber optics, confocal back or forward fluorescence detection in single or array configurations, and the like.

Detection may be by any of the known methods associated with the analysis of capillary electrophoresis columns including the methods shown in U.S. Pat. Nos. 5,560,811 (column 11, lines 19-30), U.S. Pat. Nos. 4,675,300, 4,274,240 and 5,324,401, the relevant disclosures of which are incorporated herein by reference.

Those skilled in the electrophoresis arts will recognize a wide range of electric potentials or field strengths maybe used, for example, fields of 10 to 1000 V/cm are used with 200-600 V/cm being more typical. The upper voltage limit for commercial systems is 30 kV, with a capillary length of 40-60 cm, giving a maximum field of about 600 V/cm. For DNA, typically the capillary is coated to reduce electroosmotic flow, and the injection end of the capillary is maintained at a negative potential.

For ease of detection, the entire apparatus may be fabricated from a plastic material that is optically transparent, which generally allows light of wavelengths ranging from 180 to 1500 nm, usually 220 to 800 nm, more usually 450 to 700 nm, to have low transmission losses. Suitable materials include fused silica, plastics, quartz, glass, and so forth.

V. Electrophoretic Systems for Use of the e-tag Reagents

One embodiment of a system according to the present invention is presented in FIG. 16. This figure illustrates a system 100 for the simultaneous, multiplexed determination of a plurality of events. Each event is distinguished from the others by electrophoresis. For example, a SNP locus may be characterized using a pair of reagents, each specific for one allele of the locus. Each reagent is bonded to an e-tag moiety with a unique electrophoretic mobility and an associated label. When the reagent is combined with a sample of interest in a reaction vessel 101, the associated e-tag probe is modified in a manner that changes its electrophoretic mobility if its specific target is present. After the reaction, the mixture is moved 102 onto an electrophoretic device 103 for separation of the e-tag reporter products contained in the mixture. A power control box 104 is used in conjunction with the device to control injection of the sample into the separation channel 105. Each e-tag reporter species migrates down the separation channel of the device with a mobility unique to that tag, moving past a detector 106 that monitors its presence by its associated label. The data collected by the detector is sent to a data processor 107, which determines the presence of each SNP allele in the sample based on the mobility of its corresponding e-tag reporter.

In another example, a group of SNP loci or other sequences may be monitored in a multiplexed reaction. In this case, a plurality of pairs of e-tag reagents corresponding to the target sequences are combined with a sample in a single reaction vessel under conditions where the e-tag reporter is released from at least a portion of the target oligonucleotide sequence to which it is bonded when a pair is bonded to its target. The e-tag reporters are either labeled for detection or the label is added by means of a reactive functionality present on the e-tag moiety. The labeled e-tag products of the reaction are resolved from one another on the electrophoretic device, and again are monitored as they move past the detector. The level of multiplexing possible in this system is limited only by the degree of resolution that can be obtained between a designated set of e-tag reporters on the electrophoretic device.

An additional degree of flexibility can be conferred on the assay by the stage at which the e-tag reporters are labeled. As described above, each e-tag moiety may already contain a detectable label when introduced into the reaction. Alternatively, an e-tag moiety may contain a functionality allowing it to bind to a label after reaction with the sample is complete (FIG. 16; 108). In this embodiment, an e-tag probe comprising a functionality for binding to a detectable label is combined with a sample (FIG. 16; 101). After a reaction to modify the mobility of the e-tag probe if its target is present in the sample, additional reagents are combined in a sample vessel (FIG. 16; 109) with the products of the first reaction, which will react with the modified e-tag reporter(s) to add a detectable label.

VI. Compositions Containing Interactive Functionalities

In another aspect, the present invention is directed to biopolymers and biopolymer precursors that comprise an interactive functionality as defined herein. Preferably, the interactive functionality comprises binding groups in a cis and/or coplanar relationship. The cis and/or coplanar relationship may result from the groups being disposed on an unsaturated bond, such as a carbon-carbon double bond or a ring having a geometry that results in a cis relationship, such as an aromatic ring or rigid aliphatic ring, including fused systems. The coplanar relationship may be found, for example, in a fused ring system where each functionality is disposed on a different ring in, for example, a 1,8-naphthyl system.

The interactive functionalities in accordance with the present invention comprise groups that can interact with another moiety by conjugation or complexation. Examples include hydroxy, amino, alkylamino, arylamino, thio, aldehyde, oxo keto), carboxyl, carboxamide, N-alkyl- or N-aryl-substituted carboxamide, and combinations thereof. Such interactive functionalities include, for example, 1,2-diols, 1,3-diols, 1,2-aminoalcohols, 1,3-aminoalcohols, 1,2-hydroxy acids, 1,3-hydroxy acids, 1,2-hydroxy acid amides, 1,3-hydroxy acid amides, dioximes, and so forth.

The interactive functionalities may be part of an organic moiety having about 6 to about 100 atoms selected from carbon, oxygen, nitrogen and sulfur. Usually, the organic moiety has from 2 to about 60 carbon atoms, more usually, from 5 to about 50 carbon atoms, and at least one, up to ten, and typically two ti four of the aforementioned interactive groups. Specific examples of organic moieties having an interactive functionality include ortho-hydroxybenzenes (catechols), ortho-hydroxy benzohydroxamic acids, ortho-hydroxybenzoic acids, ortho-hydroxybenzamides, imidazolyl phenols, dihydroxyfumaric acids, hydroxy pyridine aldehydes, and hydroxyanthraquinone dioximes. Examples of particular compounds that may be incorporated into the biopolymers or biopolymer monomer precursors include catechol, salicylamide, N-phenylsalicylamidine, 2-(4′imidazolyl)phenol, 1,8-dihydroxy naphthalene, dihydroxyfumaric acid, salicylaldehyde, 3-hydroxypyridine-4-aldehyde, pyridoxamine, 1-hydroxyanthraquinone dioxime, disalicylimide, and so forth. Synthetic biopolymers include peptides and polynucleotides, as well as such compounds composed of or containing amino acid analogs or non-amino acid groups, or nucleotide analogs or non-nucleotide groups, as well as polysaccharides, and the like. The term “polynucleotide” also includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in base-pairing interactions. The term peptide includes proteins, polypeptides, and oligopeptides.

Oligonucleotides are recognized as a class of polynucleotides having a relatively short length. Oligonucleotides are at least about 2 nucleotides, usually about 5 to about 100 nucleotides, in length.

The biopolymer precursors are generally monomer units from which the biopolymers are formed; e.g. nucleoside monomers or amino acids. In one embodiment, the biopolymer precursors are synthetic nucleotides or nuclesides.

In general, the interactive functionalities are incorporated into the biopolymer in a manner and at a position that does not interfere with the binding capabilities and related properties of the biopolymer. The biopolymer or biopolymer precursor may comprise 1 to about 10, or, in selected embodiments, 2 to about 8, or 3 to 6, interactive functionalities. They may be incorporated into the biopolymer, or into the biopolymer precursor, by conjugation chemistries known in the art. Typical conjugation functionalities include, for example, amino, thiol, aldehydes, and activated esters such as NHS-esters.

The following discussion is directed to synthetic oligonucleotide and nucleosides of the invention. Those skilled in the art, in view of the disclosure herein, will be able to employ the present technology in the preparation and use of other types of biopolymers and their precursors.

One embodiment of the invention is directed to nucleosides that comprise a ribose sugar, a base, such as a purine or pyrimidine base or modified derivatives thereof, and at least one interactive functionality. Another embodiment of the invention is directed to polynucleotides comprising such nucleosides.

An organic moiety comprising the interactive functionality may be incorporated into e.g. the sugar moiety or the base moiety of a nucleoside. An example of a synthetic nucleoside of the invention is shown below. In this case, the interactive functionality, represented by the various embodiments of Y, is included in the base moiety (R₄) or the 2′ position of the sugar moiety (R₃). 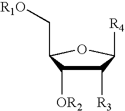
In the above formula, R¹ is H, dimethoxytrityl (DMT), triphosphate ester, diphosphate ester, or monophosphate ester;

R² is H or phosphoramidite;

R³ is H, OH, ODMT or OX(Y)p;

and R⁴ is one of:

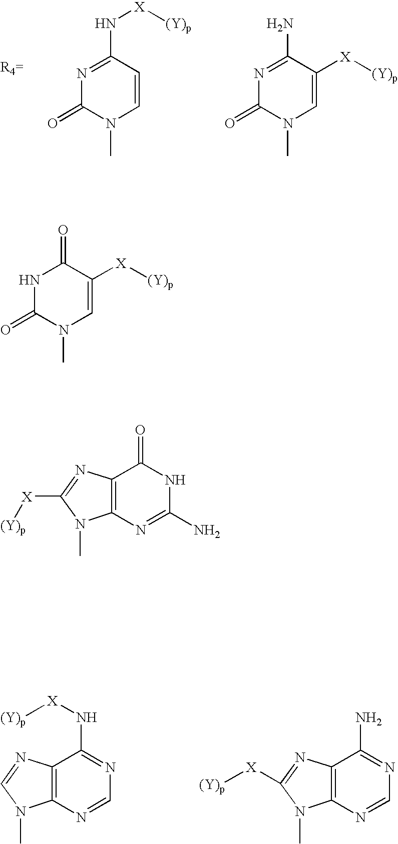

In R⁴, p is 0 to 3, usually I to 3, and p in at least one of R³ or R⁴ is 1. X is a bond or a linking group, and Y is Ya or Yb, as shown below:

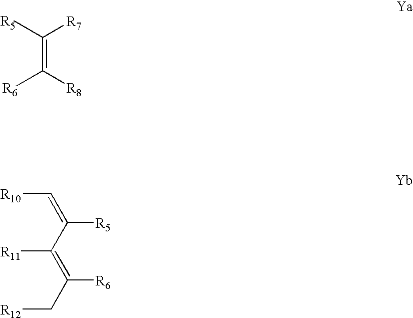

In Ya, R⁵ and R⁶ are independently hydroxy, amino, substituted amino, carbonyl (as in keto, aldehyde and the like), carboxamide, or N-hydroxy amide, where any of these can include a protecting group such as, for example, acetyl, ethyl, isobutyryl, or t-butyryl. R⁷ and R⁸ are independently carboxy or cyano or together form one or more rings, usually 1 to 3 rings, which are usually fused when more than one ring, wherein each of the rings may comprise 5 to 10 atoms, usually 5-6 atoms, selected from carbon, nitrogen, oxygen, and sulfur, where at most two ring atoms are non-carbon. The rings may comprise one or more double bonds in addition to the double bond of the above formula. Examples of rings include phenyl, anthracyl, pyridinyl, and the like, wherein the atoms of the rings may be substituted with one or more groups such as, for example, alkyl, hydroxy, nitro, oxo, and the like. X is linked to Ya through R⁷ or R⁸.

In Yb, R⁵ and R⁶ are as described above, and R¹⁰ and R¹¹ and R¹² are independently carboxyl or cyano or together form one or more rings, usually 2 to 3 rings, which, when more than one ring, are usually fused, wherein each ring may comprise 5. to 10 atoms, usually 5-6 atoms, selected from carbon, nitrogen, oxygen, and sulfur, where at most two ring atoms are non-carbon. The rings may comprise one or more double bonds in addition to the double bond of the above formula. Examples of rings include naphthyl, anthracyl, phenanthryl, and the like, wherein the atoms of the rings may be substituted with one or more moieties such as, for example, alkyl, hydroxy, nitro, oxo, and the like. X is linked to Y through R¹⁰ or R¹¹ or R¹².

When X is a linking group, it typically comprises a chain of 1-100, usually 1-70, preferably 1-50, and more preferably 2-30 atoms, each independently selected from carbon, oxygen, sulfur, nitrogen, and phosphorous. The number of heteroatoms in the chain is normally 0-20, usually 1-15, preferably 2-6. The atoms in the chain maybe substituted with atoms other than hydrogen. As a general rule, the length of a particular lining group X can be selected arbitrarily to provide for convenience of synthesis.

The linking group may comprise functional groups such as, e.g., ether groups, carbonyl functionalities, both oxocarbonyl, e.g., aldehyde, and non-oxocarbonyl (including nitrogen and sulfur analogs) e.g., carboxy, amidine, amidate, thiocarboxy and thionocarboxy. Common functionalities resulting from synthetic formation of a covalent bond between the linking group and the moiety to which it is linked include ether, alkylamine, amidine, thioamide, urea, thiourea, guanidine, azo, thioether, carboxylate, sulfonate, and phosphate esters, amides, and thioesters. The linking group may comprise one or more polyoxyalkylene groups such as, for example, polyoxyethylene groups comprising from about 1 to about 100 oxyalkylene groups.

Y may be attached to the terminus of the linking group or, where more than one Y is present, each Y may be attached at a point along the chain of the linking group, usually through the intermediacy of a functionality for binding Y to the chain.

Another specific embodiment of nucleosides that may be employed in the synthesis of polynucleotide e-tag probes is represented by the formula: 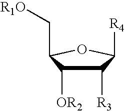
wherein R¹, R², R³ and R⁴, X and p are as described above, and
wherein Y is Ya′ or Yb′ as shown below:

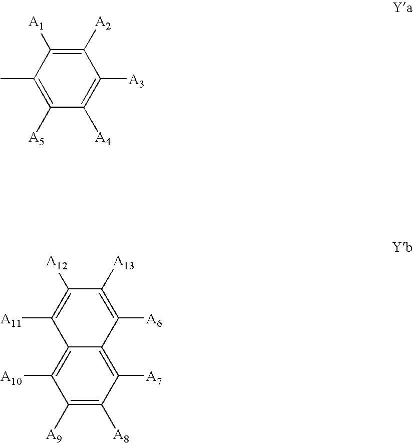

In Y′a, A1 to A⁵ are independently selected from hydrogen, hydroxy, amino, substituted amino, carbonyl (as in aldehyde, keto and the like), carboxamide, and N-hydroxy amide, any of which may be bound to a protecting group such as, for example, acetyl, ethyl, isobutyryl, or t-butyryl, wherein at least two adjacent members of A¹ to A⁵, preferably at least two adjacent members of A² to A⁴, are not hydrogen. In Y′b, A⁶ and A⁷ are independently selected from hydroxy, amino, substituted amino, carbonyl (as in aldehyde, keto and the like), carboxamide, and N-hydroxy amide, any of which may be protecting group as above. One of A⁸ to A¹³ is linked to X, and the others are independently selected from the groups disclosed for A⁶ and A⁷ above.

Other specific embodiments of reagents that may be used to prepare e-tag probes are set forth in the following formulas, depicting a protected phosphoramidite reagent and derivatized solid support, respectively:

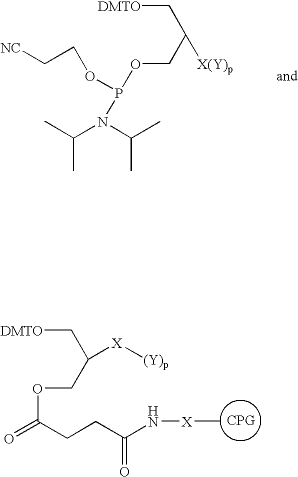

In these structures, X and Y are as described above and CPG represents a solid support, preferably controlled pore glass. Specific examples of compounds of the above formulas are set forth in FIGS. 35 and 49.

FIGS. 52 and 53 depict various catechol derivatives that comprise reactive functionalities for use in linking the compound with a nucleoside or oligonucleotide, by reaction with a corresponding functionality on the nucleoside or oligonucleotide. In the figures, X′ preferably represents alkylene, e.g. methylene, 0, NH, S, or combinations thereof, n is 1 to 12, R is C₁-C₁₂ alkyl, and Y″ is Cl, Br, or I. The catechol ring may be further substituted with one or more moieties such as, for example, alkyl, nitro, hydroxyl, and so forth.

Certain specific embodiments of dNIP's of catechol-derivatized dC, dT, dA, dG and dU are shown in FIGS. 50A-50E. Furthermore, certain specific embodiments of phosphoramidites of catechol-derivatized dC, dT, dA, dG and dU are shown in FIGS. 51A-51E. The dNT's and the phosphoramidites maybe synthesized according to procedures well known in the art, employing the synthetic techniques described herein for incorporation of the catechol moieties, as illustrated, for example, in FIGS. 58-62.

The derivatized monomers may be employed in the synthesis of polynucleotides and oligonucleotides comprising one or more catechol moieties, using oligonucleotide synthesis protocols known in the art, e.g. phosphate coupling, either direct phosphate coupling or via a phosphate precursor such as a phosphite. Such coupling thus includes the use of amidite (phosphoramidite), phosphodiester, phosphotriester, H-phosphonate, or phosphite halide intermediates. In a preferred approach, standard phosphoramidite chemistry is employed, preferably in the solid phase. In a typical procedure, a support bound monomer with a free 5′-hydroxyl group is reacted with a 5′-protected deoxyribonucleoside-3′-O-(N,N-diisopropylamino)phosphoramidite. The 5′ protecting group is removed after the coupling reaction, and the procedure is repeated with additional protected nucleotides until synthesis of the desired polynucleotide is complete. Oxidation can be effected with iodine to give phosphates or with sulfur to give phosphorothioates.

Incorporation of an organic moiety comprising a cis-diol into an oligonucleotide is also described in Cao et al., Chem. Pharm. Bull. (2000) 48(11) 1745.

The interactive functionality often requires protection during automated DNA synthesis. The protecting group is preferably acid-stable. Examples of protecting groups for diols, by way of illustration and not limitation, are carbonates, esters, and allelic-based protecting groups, which are removed during ammonolysis or via treatment with specialized reagents such as palladium in the case of allyloxy carbonyl groups. A dithiol may be protected as a disulfide or thioester. Other protecting groups for various functionalities are known in the art; see e.g. Kocienski, cited above.

In another aspect, the invention provides a method of purifying a synthetic oligonucleotide of predetermined length, synthesized according to the procedures described above, by contacting the product mixture with a solid support comprising the capture agent. The synthetic oligonucleotide can thus be segregated from various reactants and by-products typically present in such a reaction mixture. The support may be a particulate support contained in, for example, a chromatography column. The reaction mixture is passed through the column and the oligonucleotide of interest is retained therein. After suitable washing, the support is contacted with a reagent that releases the oligonucleotide of interest. The nature of the releasing agent depends on the nature of the complexing reagent.

The support is preferably glass whose surface has been chemically activated to support binding or synthesis thereon, e.g. Bioglass™ and the like. The support may alternatively be made from materials such as inorganic powders, e.g., silica, magnesium sulfate, and alumina; natural polymeric materials, particularly cellulosic materials such as fiber-containing papers, e.g., filter paper, chromatographic paper, etc.; synthetic or modified naturally occurring polymers, such as nitrocellulose, cellulose acetate, poly (vinyl chloride), polyacrylamide, crosslinked dextran, agarose, polyacrylate, polyethylene, polypropylene, poly(4-methylbutene), polystyrene, polymethacrylate, poly(ethylene terephthalate), nylon, poly(vinyl butyrate), etc.; ceramics, metals, and the like.

Other preferred support materials include dextran, aminodextran, cellulose, agarose, polyacrylamide, polymethylmethacrylate, silica, glass, and polymers that include a complexing agent as an integral part of a polymer backbone. In one embodiment, the complexing agent is a boronic acid moiety. The boronic acid moiety is typically a substituted boronic acid, wherein the substituent is an organic moiety, either aliphatic or aromatic, having about 2 to about 100 atoms, selected from the group consisting of carbon, oxygen, nitrogen, and sulfur; usually the organic moiety has from about 2 to about 60 carbon atoms. Such a substituted boronic acid may be represented as R-B(OH)₂, where R is the organic moiety.

An important consideration regarding the boronic acid moiety is its acidity. In general, the higher the acidity of the boronic acid moiety, the better is the ability to.: complex with the boronic acid complexing moiety and to to separate biomolecules under physiological conditions, e.g at a pH of about 7A4. Desirably, the pKa of the boronic acid moiety is <11, preferably <9. Accordingly, substituents on the boron that enhance the acidity over that of boronic acid are preferred. Aromatic substituents on the boron are preferred; to enhance acidity, the aromatic substituent preferably contains one or more electron-withdrawing groups such as nitro, cyano, carboxyl, keto, and the like. Specific boronic acid moieties include, by way of illustration and not limitation, phenyl boronic acid and (3-aminophenyl) boronic acid. Other examples of specific phenylboronic acids are depicted in FIGS. 54-55, and still other examples may be found in U.S. Pat. Nos. 5,623,055, 5,876,938, 6,013,783 and 5,831,045, the relevant disclosures of which are incorporated herein by reference.

In various embodiments, the support is a boronate gel, such as, for example, Affi-Gel 601® (BioRad Laboratories, Richmond, Calif.), polyacrylamide bulk gel (Aldrich Chemical Co., Milwaukee, Wis.), agarose bulk gel (Amicon Corp., Danvers, Mass.), polymethylmethacrylate bulk gel (Toyo-Soda Corp., Tokyo, Japan), or agarose or polyacrylamide bulk gel (Pierce Chemical Co., Rockford Ill.). Commercially available boronate supports include a pre-packed SPE silica tube from Analytichem Intl., Harbor City, Calif., a high performance ligand affinity chromatography (HPLAC) column, either agarose or polyacrylamide, from Pierce Chemical Co., or polymethylmethacrylate from Toyo-Soda Corp. Structures of some typical commercially available boronate affinity matrices are shown in FIG. 55. A boronate support can also be prepared by linking a boronic acid moiety to a preformed support. In another approach, a boronic acid moiety may be incorporated into a polymeric support during the polymerization step.

VII. Kits for Use of the E-Tag Reagents

As a matter of convenience, predetermined amounts of reagents employed in the present invention can be provided in a kit in packaged combination. One exemplary kit for SNP detection can comprise in packaged combination: an oligonucleotide primer for each suspected target polynucleotide, wherein each of said primers is hybridizable to a first sequence of the respective polynucleotide if present, a template-dependent polynucleotide polymerase, nucleoside triphosphates, and a set of SNP detection sequences, each having a fluorescent label at its 5′-end and having a sequence at its 3′-end that is hybridizable to a respective target polynucleotide, wherein each of the electrophoretic labels is cleavable from the SNP detection sequence.

The kit may further comprise a device for conducting capillary electrophoresis, as well as a template-dependent polynucleotide polymerase having 5′ to 3′ exonuclease activity. The kit can further include various buffered media, some of which may contain one or more of the above reagents.

The relative amounts of the various reagents in the kits can be varied widely to provide for concentrations of the reagents necessary to achieve the objects of the present invention. Under appropriate circumstances, one or more of the reagents in the kit can be provided as a dry powder, usually lyophilized, including excipients, which on dissolution will provide for a reagent solution having the appropriate concentrations for performing a method or assay in accordance with the present invention. Each reagent can be packaged in a separate container, or some reagents can be combined in one container, where cross-reactivity and shelf life permit. For example, the dNIs, the oligonucleotide pairs, and optionally the polymerase, may be included in a single container, which may also include an appropriate amount of buffer. The kits may also include a written description of a method in accordance with the present invention as described above.

Such kits are useful in detecting the presence and/or amount of each of a plurality of target molecules. In this aspect, the kit comprises in packaged combination a plurality of probes. Each of the probes is capable of target-specific binding to a binding site on a selected target. Each of the probes has a unique cleavable reporter group and an interactive functionality adjacent the reporter group positioned in the probe such that the interactive functionalities do not form part of the reporter group upon release thereof. The cleavable reporter group in each probe includes (i) a cleavable moiety that is susceptible to cleavage, and (ii) an electrophoretic tag having a detectable reporter moiety and a unique electrophoretic mobility, which allows the tag to be uniquely identified among all other tags associated with the plurality of probes. The organic. moiety comprising interactive functionalities is effective in isolating the signal produced by released reporter groups from the signal produced by intact probes and other fragments thereof. The kit may also include (b) one or more binding agents capable of binding to a further binding site on the target molecules, e.g. oligonucleotide primers.

The kit may also comprise building blocks for preparation of e-tag probes in situ or have assembled e-tag moieties for direct bonding to the binding compound. For preparing the e-tag probes in situ during the synthesis of oligonucleotides, phosphoramidites or phosphates are provided, where the esters include alkyl groups, particularly of from 1 to 3 carbon atoms, and cyanoethyl groups, while for the phosphoramidite, dialkylamino, where the alkyl groups are of from 1-4 carbon atoms, while the other group would be a protected hydroxy, where the protecting group is common to oligonucleotide synthesis, e.g. dimethoxytrityl. For large numbers of e-tag probes, that is, 20 or more, one kit would supply at least 3 each of mass-modifying regions and charge-modifying regions, each having at least the phosphate linking group and a protected hydroxyl. The two functional groups may be separated by 2 or more atoms, usually not more than about 60 atoms, and may be vicinal (α,β to α,ω). The nature of the compounds has been discussed previously. In the simplest case, the phosphorous acid derivative would serve as the charge-modifying region, so that the mass-modifying region and the charge-modifying region would be added as a single group. In addition, at least 2 detectable regions may be employed, which would be a fluorescer having the phosphate linker and other functionalities protected for purposes of the synthesis. Alternatively, instead of having the detection region the terminal region, where the detectable region allows for the presence of two functionalities that can be used for linking, one of the other regions may serve as the terminal region. Also, one of the regions may be conveniently linked to a mono- or dinucleotide for direct linking to the oligonucleotide chain, where cleavage will occur at the 3′ site of the nucleotide attached to the e-tag reporter. By using tri- or tetra-substituted groups, a detectable region is employed that provides the pair for energy transfer. Only one or two different energy transfer agents are needed, while having a plurality of emitting agents to greatly expand the number of different e-tag reporters. The kits preferably include components sufficient to prepare at least 10, usually at least 20, and frequently at least 50 different e-tag reporters that can be separated by their mobility.

Other convenient chemistries, particularly chemistries that are automated, may be used in place of or in addition to phosphate chemistry. These include, in place of phosphorous acid and protected alcohol, one can use carboxy and alcohol or amino, activated olefin and thiol, amino and oxo-carbonyl, particularly with reductive amination, an hydroxy with an active halide or another hydroxy to form an ether, and the like.

Another aspect of the present invention relates to kits useful for synthesizing an oligonucleotide having one or more interactive functionalities incorporated therein. The kit may comprise in packaged combination a biopolymer precursor, such as a nucleoside, which comprises one or more interactive functionalities. The nucleoside may be in the form of a phosphoramidite reagent. Other reagents may be included for the synthesis of an oligonucleotide such as, for example, nucleoside reagents that do not comprise interactive functionalities. The reagents can be provided in packaged combination, in the same or separate containers. The reagents may each be in separate containers or various reagents can be combined in one or more containers depending on the cross-reactivity and stability of the reagents. The relative amounts of the various reagents in the kits can be varied widely to provide for concentrations of the reagents that are necessary for their use. The kit can further include a written description of a method employing the packaged reagents or the products of the reaction of the packaged reagents.

### EXAMPLES

The invention is demonstrated further by the following illustrative examples. Parts and percentages are by weight unless otherwise indicated. Temperatures are in degrees Centigrade (° C.) unless otherwise specified. The following preparations and examples illustrate the invention but are not intended to limit its scope. Unless otherwise indicated, oligonucleotides and peptides used in the following examples were prepared by synthesis using an automated synthesizer and were purified by gel electrophoresis or HPLC.

The following abbreviations have the meanings set forth below:
Tris HCl—Tris(hydroxymethyl)aminomethane-HCl (a 10× solution) from BioWhittaker, Walkersville, Md.
BSA—bovine serum albumin from Sigma Chemical Company, St. Louis Mo.
EDTA—ethylene diamine tetra-acetate from Sigma Chemical Company
bp—base pairs
TET—tetrachlorofluorescein
FAM—fluorescein
TAMRA—tetaamethyl rhodamine
EOF—electrbosmotic flow
   Reagents

TET and TAMRA were purchased from Perlin Elmer (Foster City, Calif.) as were conjugates of TET, FAM and TAMRA with oligonucleotides.
Master Mix (2×): 20 mM Tris-HCl, 2.0 mM EDTA, pH 8.0 (8% Glycerol),
10 mM MgCl₂, DATP 400 μM, dCTP 400 μM, dGTP 400 μM,
dUTP 400 μl AmpliTaq Golds 0.1 U/μL (from Perkin Elmer), Amperase UTNO 0.02 U/μL (from Perkin Elmer)

Probes and Primers: (10×)

| | |
|---|---|
| Forward Primer: | |
| 3.5 μM 5′-TCA CCA CAT CCC AGT G-3′ | (SEQ ID NO: 1) |
| Reverse Primer: | |
| 2.0 μM 5′-GAG GGA GGTTTG GCTG-3′ | (SEQ ID NO: 2) |

Plasmid Allele 1 Probe: 2.0 μM (200 nM per reaction)
5′ TET-CCA GCA ACC AAT GAT GCC CGT T-TAMRA-3′ (SEQ ID NO:3)
Plasmid Allele 2 Probe: 2.0 μM (200 nM per reaction)
5′ FAM-CCA GCA AGC ACT GAT GCC TGT T-TAMRA-3′ (SEQ ID NO:4)
Target DNA:
Plasmid Allele-1: 10 fg/μL=approximately 1000 copies/μL
Plasmid Allele-2: 10 fg/μL=approximately 1000 copies/μL

### Example 1

### Synthesis of Elements of E-Tag Probes

A. Synthesis of 6-Carboxyfluorescein Phosphoramidite Derivatives

To a solution of 6-carboxyfluorescein (0.5 g, 1.32 mmol) in dry pyridine (5 mL) was added drop wise, isobutyric anhydride (0.55 mL, 3.3 mmol). The reaction was allowed to stir at room temperature under an atmosphere of nitrogen for 3 h. After removal of pyridine in vacuo the residue was redissolved in ethyl acetate (150 mL) and washed with water (150 mL). The organic layer was separated, dried over Na₂SO₄, filtered, and concentrated in vacuo to yield a brownish residue. This material was dissolved in CH₂Cl₂ (5 mL) after which N-hydroxy succinimide (0.23 g, 2.0 mmol) and dicyclohexylcarbodiimide (0.41 g, 1.32 mmol) were added. The reaction was allowed to stir at room temperature for 3 h and then filtered through a fritted funnel to remove the white solid, which had formed. To the filtrate was added aminoethanol (0.12 mL, 2.0 mmol) dissolved in 1 mL of CH₂Cl₂. After 3 h the reaction was again filtered to remove a solid that had formed, and then diluted with additional CH₂Cl₂ (50 mL). The solution was washed with water (150 mL) and then separated. The organic layer was dried over Na₂SO₄, filtered, and concentrated in vacuo to yield a white foam (0.7 g, 95%, 3 steps). ¹HNMR: (DMSO), 8.68 (t, 1H), 8.21 (d, 1H), 8.14 (d, 1H), 7.83 (s, 1H), 7.31 (s, 2H), 6.95 (s, 4H), 4.69 (t, 1H), 3.45 (q, 2H1), 3.25 (q, 2H), 2.84 (h, 2H), 1.25 (d, 12H). Mass (LR FAB⁺) calculated for C₃₁H₂₉NO₉ (M+H⁺) 559.2, found: 560.

B. Synthesis of Modified Fluorescein Phosphoramidites

B1. Pivaloyl protected carboxvfluorescein: Into a 50 mL round bottom flask was placed 5(6)-carboxyfluorescein (0.94 g, 2.5 mmol), potassium carbonate (1.0 g, 7.5 mmol) and 20 mL of dry DMP. The reaction was stirred under nitrogen for 10 min, after which trimethylacetic anhydride (1.1 mL, 5.5 mmol) was added via syringe. The reaction was stirred at room temperature overnight, and then filtered to remove excess potassium carbonate and finally poured into 50 mL of 10% HCl. A sticky yellow solid precipitated out of solution. The aqueous solution was decanted off and the residual solid was dissolved in 10 mL of methanol. Drop wise addition of this solution to 10% HCl yielded a fine yellow precipitate, which was filtered and air dried to yield an off white solid (0.88 g, 62%). TLC (45:45:10 of Hxn:EtOAc:MeOH).

B2. NHS ester of protected pivaloyl carboxyfluorescein: Into a 200 mL round bottom flask was placed the protected carboxyfluorescein (2.77 g, 5.1 mmol) and 50 mL of dichloromethane. N-hydroxysuccinimide (0.88 g, 7.6 mmol) and dicyclohexylcarbodiimide (1.57 g, 7.6 nmol) were added and the reaction was stirred at room temperature for 3 hours. The reaction was then filtered to remove the precipitated dicyclohexyl urea byproduct and reduced to approx. 10 mL of solvent in vacuo. Drop wise addition of hexanes with cooling produced a yellow-orange colored solid, which was triturated with hexanes, filtered and air-dried to yield 3.17 g (95%) of product. TLC (45:45:10 of Hxn:EtOAc:MeOH)

B3. Alcohol: Into a 100 mL round bottom flask was placed the NHS ester (0.86 g, 1.34 mmol) and 25 mL of dichloromethane. The solution was stirred under nitrogen after which aminoethanol (81 mL, 1 eq) was added via syringe. The reaction was monitored by TLC (45:45:10 Hxn, EtOAc, MeOH) and was found to be complete after 10 min. The dichloromethane was then removed in vacuo and the residue dissolved in EtOAc, filtered and absorbed onto 1 g of silica gel. This was bedded onto a 50 g silica column and eluted with Hxn:EtOAc:MeOH (9:9:1) to give 125 mg (20%) of clean product.

B4. Phosphoramidite: Into a 10 mL round bottom flask containing 125mg of the alcohol was added 5 mL of dichloromethane. Diisopropyl ethylamine (139 μL, 0.8 mmol) was added via syringe. The colorless solution turned bright yellow. 2-cyanoethyl diisopropylchlorophosphoramidite (81 μL, 0.34 mmol) was added via syringe and the solution immediately went colorless. After 1 hour TLC (45:45: 10 of Hxn:EtOAc:TEA) showed the reaction was complete with the formation of two closely eluting isomers. Material was purified on a silica column (45:45:10 of Hxn:EtOAc:TEA) isolating both isomers together and yielding 130 mg (85%).

B5. Carboxylic acid: Into a 4 mL vial was placed 12-aminododecanoic acid (0.1 g, 0.5 mmol) and 2 mL of pyridine. To this suspension was added chlorotrimethyl silane (69 μL, 1.1 eq) via syringe. After all material dissolved (10 min) NHS ester (210 mg, 0.66 eq) was added. The reaction was stirred at room temperature overnight and then poured into water to precipitate a yellow solid, which was filtered, washed with water, and air-dried. TLC (45:45:10 of Hxn:EtOAc:MeOH) shows a mixture of two isomers.

General Procedure for Remaining Syntheses. The carboxylic acid formed as described above is activated by NHS ester formation with 1.5 eq each of N-hydroxysuccinimide and dicyclohexylcarbodimide in dichloromethane. After filtration of the resulting dicyclohexylurea, treatment with 1 eq of varying amino alcohols will effect amide bond formation and result in a terminal alcohol. Phosphitylation using standard conditions described above will provide the phosphoramidite.

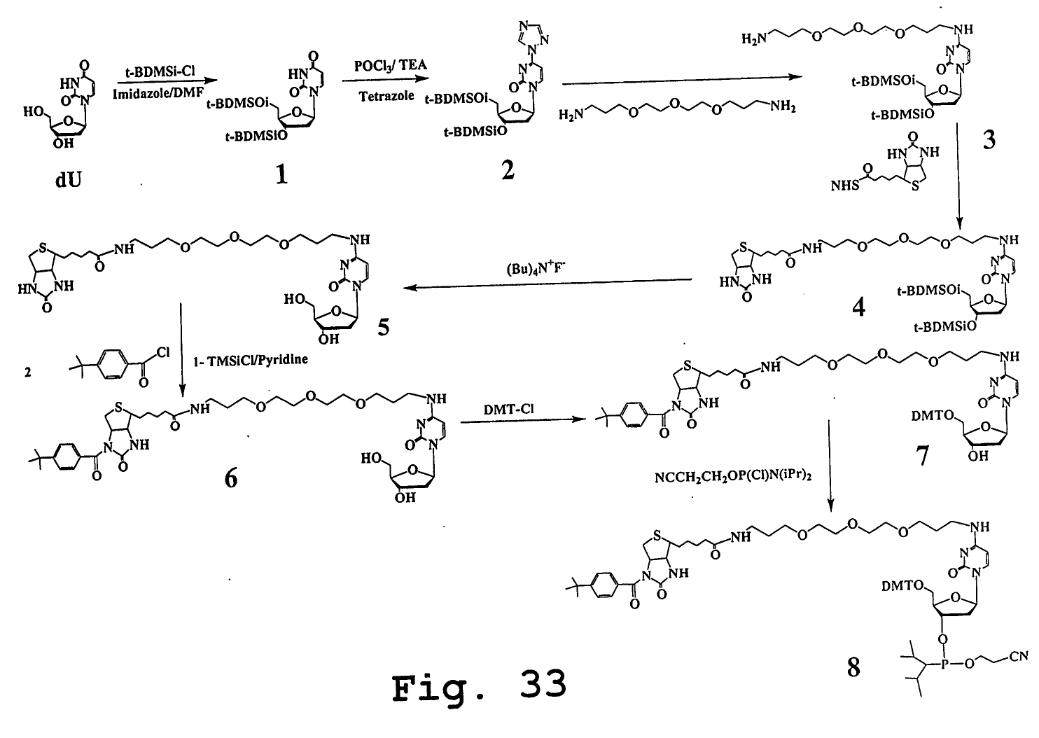

C. Synthesis of Biotinilated 2′-Deoxycvtosine Phosphoramidite (FIG. 33)

C1. Synthesis of 3′,5′-O-di-t-butyldimethylsilyl-2′-Deoxyguridine(l): 2′-deoxyuridine (4 gm, 17.5 mmol) and imidazole (3.47 gm, 52.5 mmol) were dissolved in 30 ml of dry DMF and t-butyldimethyl-silyl chloride (7.87 gm, 52.5 mmol) added to the stirring solution at room temperature. After 3 hrs, TLC on silica gel (10% MeOH+90% CH₂Cl₂) showed that all starting material had been converted to a new compound with higher R_{f}. The solution was concentrated into a small volume; about 200 ml of ether was then added and washed three times with saturated aqueous NaCl solution. The organic layer was dried over anhydrous Na₂SO₄, and the filtrate was evaporated to give a colorless gummy material that converted to a white solid product (eight g, 100%). This product was identified with HNMR and ES-MS.

C2. Synthesis of 3′,5′-O-di-t-butyldimethylsilyl-N⁴-(1,2,4-triazolo)-2′-Deoxycytidine (2): 1,2,4Triazole (19.45 g, 282 mmol) was suspended in 300 ml of anhydrous CH₃CN at 0° C., 8 ml of POCl₃, then 50 ml of triethylamine was added slowly in 5 min. After an hour, 3′,5′-O-di-t-butyldimethylsilyl-2′-deoxyuridine (Compound 1) (9 gm, 19.7 mmol) was dissolved in 200 ml of dry CH₃CN and added to the reaction over 20 min. After string the reaction for 16 hours at RT, TLC (100% ether) showed that all starting material was converted to a new compound with lower Rf. The reaction mixture was filtered, reduced the volume of CH₃CN, diluted with ethyl acetate and washed with saturated aqueous NaHCO₃ then twice with saturated aqueous NaCl. The organic layer was dried over anhydrous Na₂SO₄ and the solvent was evaporated, co-vaporated from toluene to give a yellow solid product (10 g, 100%/o). This product was dentified with III and ES-MS.

C3. Synthesis of 3′,5′-O-di-t-butyldimethylsilyl-N⁴-(4,7,10-trioxa-1-tridecaneamino)-2′-deoxycytidine (3): 4,7,10-Trioxa-1,13-tridecanediamine (10.44 gm, 47.4 mmol) was dissolved in 100 ml dioxane, then 3′,5′-O-di-t-butyldimethylsilyl-4-(1,2,4-triazolo)-2′-deoxycytidine (Compound 2) (8.03 gm, 15.8 mmol) was dissolved in 200 ml of dioxane (heated to about 50 C and cooling it dawn to RT) and added drop wise in 10 min, to the solution of 4,7,10-Trioxa-1,13-tridecanediamine with vigorous stirring at RT. After 5 hrs, TLC on silica gel showed that all starting material was converted to a new product with lower Rf, the resulting mixture was evaporated to dryness. The residue was dissolved in dichloromethane and washed twice with 5% sodium bicarbonate solution and saturated sodium chloride solution. The organic layer was dried over sodium sulphate, filtered and evaporated to dryness to give a yellow gummy product (7.87 g). The product was purified on a silica gel column eluted with a gradient of 0 to 10% methanol in dichloromethane with 1% triethylamine. The product was, obtained as a yellowish gum (5.66 g, 54%). This product was identified with HNMR and ES-MS.

C4. Synthesis of 3′,5′-O-di-t-butyldimethylsilyl-4-N-(4,7,10-trioxa-1-tridecaneaminobiotin)-2′-deoxycytidine (4): 3′,5′-O-Di-t-butyldimethylsilyl-4-N-(4,7,10-trioxa-1-tridecaneamino)-2′-deoxycytidine (Compound 3) (2.657 gm, 4.43 mmol) and Biotin-NHS ester (1.814 gm, 5.316 mmol) were dissolved in 20 mL of dry DMF and about 1 mL of triethylamine was added. After stirring the reaction mixture for 4 hrs at RT, the reaction was stopped by evaporating all DMF to give a yellow gum material (4.36 g). This material was dissolved in dichloromethane and washed three times with saturated solution of NaCl, dried over sodium sulphate and evaporated to dryness. TLC on silica gel (5% MeOH+1% TEA+94% CH₂Cl₂) indicated the formation of a new product that was higher Rf. This product was purified with column chromatography on silica gel using (99% CH₂Cl₂+1% TEA) to (1% MeOH+1% TEA+98% CH₂Cl₂) to yield a yellow foamy product (2.13 g, 60%). This product was identified with HNMR and ES-MS.

C5. Svnthesis of 4-N-(4,7,10-trioxa-1-tridecaneaminobiotin)-2′-deoxycytidine (5): 3′,5 ′-O-Di-t-butyldimethylsilyl-4-N-(4,7,10-trioxa-1-tridecaneaminobiotin)-2′-deoxycytidine (Compound 4) (1.6 gm, 1.8 mmol) was dissolved in 50 mL of dry TBF, then about 5.5 mL of tetrabutylammonium fluoride in TBF was added in 2 min. while stirring at RT. After 3hrs, TLC on silica gel (10% MeOH+1% TEA+89% CH₂Cl₂) showed that a new product with lower R_{f} formed. The solvent was evaporated to give a yellow oily product. Column chromatography on silica gel eluted with (99% CH₂Cl₂+1% TEA) to (7% MeOH+1% TEA+92% CH₂Cl₂) permitted the purification of the product as a gummy colorless product (1.14 g, 97%). This product was identified with HNE and ES-MS.

C6. t-butylbenzovlation of the biotin of 4-N-(4,7,10-trioxa-1-tridecaneaminobiotin)-2′-deoxvcytidine (6): 4-N-(4,7,10-trioxa-1-tridecaneaminobiotin)-2′-deoxycytidine (Compound 5) (14.14 gm, 21.5 mmol) was dissolved in 100 mL of dry pyridine. Chlorotrimethyl silane (11.62 g, 107.6 mmol) was added and the mixture was stirred for 2 hrs at RT. 4-t-butylbenzoyl chloride (5.07 g, 25.8 mmol) was added and the mixture was stirred for another 2 hrs at RT. The reaction mixture was cooled with ice-bath and the reaction stopped by adding 50 ml of water and 50 ml of 28% aqueous ammonia solution. The solution kept stirring at RT for 20 min, then evaporated to dryness in high vacuum and finally co-evaporated twice from toluene. The material was dissolved in dichloromethane and extracted twice with 5% aqueous sodium bicarbonate solution. The organic layer was dried over sodium sulphate, evaporated to dryness, re-dissolved in dichloromethane and applied to a silica gel column. The column was eluted with gradient from 0 to 10% of methanol in dichloromethane and obtained a product as a white foam (9.4 gm, 53.5%). This product was identified with HNMR and ES-MS.

C7. Synthesis of 5′-O-(4,4′-dimethoxytriphenylmethyl-4-N-(4,7,10-trioxa-1-tridecaneaminobiotin)-2′-deoxycytidine (7): Compound 6 (10.82 gm, 13.3 mmol) was co-evaporated twice from dry pyridine, then dissolved in pyridine (100 ml) and 4,4′-dimethoxytritylchloride(DMT-Cl) (6.76 gm, 19.95 mmol) was added and the resulting mixture stirred for 3 hrs. TLC (10% MeOH+1% TEA+89% CH₂Cl₂) showed the formation of new product with higher Rf, and some starting material remained unreacted, then another amount of DMTC1 (2 gm) was added and kept stirring for 2 hrs. The reaction was stopped by adding ethanol and the mixture was stirred for 15 min. After evaporation to dryness and co-evaporation from toluene, the material was dissolved in dichloromethane. The organic layer was washed twice with 5% aqueous sodium bicarbonate solution, dried over sodium sulphate and evaporated to dryness. The product was purified on a silica column using a gradient of methanol from 0 to 5% in dichloromethane/1% TEA. The product was obtained as a white foam (4.55 gm, 31%). This product was identified with HNMR and ES-MS.

C8. Synthesis of 3′-O-[(diisopropylamine (2-cyanoethoxv) phosphino)]-5′-O-(4,4′-dimethoxytriphenylmethyl!-4-N-(4.7,10-trioxa-1-tridecaneaminobiotin)-2′-deoxyctidine (8): The 5′-DMT-Biotin-dC (Compound 7) (507 mg, 0.453 mmol) was dissolved in dry acetonitrile (30 ml) and dichloromethane (5 ml), then diisopropylamine (73 mg, 0.56 mmol), tetrazole (1.15 ml, 0.52 mmol) and 2-cyanoethyl N,N,N′N′-tetraisopropylphosphane 214 mg, 234 μL, 0.7 mmol) were added and the mixture stirred under nitrogen at RT. After 2 hrs, TLC on silica gel (45%:45%:5%:5%: of ethyl acetate:dichloromethme:triethylamine:methanol) showed that only about 30% of product was formed and about 70% of starting material was unreacted. More reagpnts were added until most of starting material was converted, with only about 5% left unreacted. The solvent was evaporated to dryness, dissolved in dry dichloromethane, washed with sodium bicarbonate solution (5%), saturated brine solution, then the organic layer was dried over sodium sulphate, evaporated to dryness. Column chromatography was carried out on silica gel using (48%:48%:4% of ethyl acetate:dichloromethane:triethylamine) to (47%:47%:5%: 1% of ethyl acetate:dichloromethane:triethylamine:methanol). The desired product was obtained as a colorless gummy product (406 mg, 70%). This material was co-evaporated three times from a mixture of dry benzene and dichloromethane, then was kept in desiccated containing P₂O₅ and NaOH pellets under vacuum for 26 hrs before used in DNA synthesis.

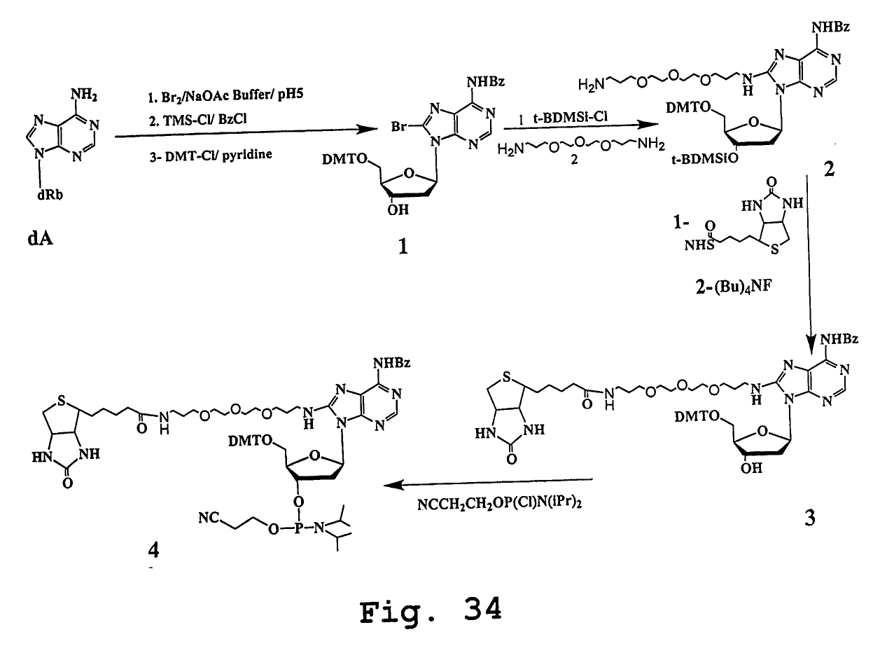

D. Synthesis of Biotinylated 2′-Deoxyadenosine Phosphoramidite (FIG. 34)

D1. Synthesis of 8-bromo-2′-deoxvadenosine (1): 2′-Deoxyadenosine (7 gm, 25.9 mmol) was dissolved in sodium acetate buffer (150 mL, 1 M, pH 5.0) by worming it to about 50° C., then was cooled dawn to 30° C., then 3 mL of bromine in 100 mL of the same buffer was added drop wise at RT for 15 min, to the reaction. After 6 hrs the TLC on silica gel (20% MEOH in CH₂Cl₂) showed that all starting material was converted to a new product. The reaction was discolored by adding some sodium metabisulfite (Na₂S₂O₅) while stirring. The color changed to a white solution, and the pH of the reaction was neutralized by adding IM NaOH. The reaction mixture was kept at 4° C. (refrigerator) for 16 hrs. The solid material was then filtered, washed with cold water, then acetone to give a solid yellow powder product (5.75 gm, 64%/o). The structure of this product was confirmed by H-NMR and ES-MS. D2. Synthesis of N⁶-benzoyl-8-bromo-5′-O-(4,4′-dimethoxytrityl)-2′-deoxyadenosine (2): 8-Bromo-2′-deoxyadenosine (Compound 1) (7.7 gm. 22.17 mmol) was dried by co-evaporation with dry pyridine and the solid was suspended in 200 ml of dry pyridine followed by the addition of 4,4′-dimethoxytriphenylmethyl chloride (DMT-Cl, 9 gm, 26.6 mmol). After stirring for 4 hrs at RT, TLC on a silica gel showed that a new product was formed and some starting material was unreacted. Another amount of, DMT-Cl (3 g) was added and stirred at RT for 2 hrs. When TLC showed that all starting material was converted to new product with a higher Rf, the reaction mixture was cooled to 0° C. and trimethylchlorosilane (12.042 gm, 14 mL, 1:10.85 mmol) was added drop wise while cooling and after 40 min while stirring benzoyl chloride (15.58 gm, 12.88 mL, 110.85 mmol) was similarly added. The reaction was allowed to react at RT over. 2hrs. The reaction was quenched by slow addition of cold water (50 ml), followed by addition of concentrated ammonia (30%, 50 ml). After 30 min, the reaction mixture was evaporated to dryness. The residue was dissolved in water, and the solution was extracted with ethyl acetate three times, the organic layer washed with saturated sodium bicarbonate solution, and then brine. The organic phase was dried over sodium sulphate, then evaporated to dryness. The product was purified by silica column chromatography, to give a yellowish solid product (6.79 g, 41.6%). The structure of this product was confirmed by H NMR and ES-MS.

D3. Synthesis of N⁶-benzoyl-8-bromo-3′-O-t-butyldimethylsilyl-5′-O-(4.4′-dimethoxytrityl)-2′-deoxyadenosine (3): 6N-Benzoyl-8-bromo-5′-O-(4,4′-dimethoxytrityl)-2′-deoxyadenosine (Compound 2) (14 gm, 19 mmol) and imidazole (1.94 gm, 28.5 mmol) were dissolved in 100 mL of dry DMF and t-butyldimethyl-silyl chloride (4.3 gm, 28.5 mmol) added to the stirring solution at room temperature. After 4 hrs, TLC on silica gel (2.5% MeOH in CH₂Cl₂) showed that all startng material had been converted to a new product with higher R_{f}. The solution was concentrated into a small volume, then about 400 mL of ether was added and washed three times with saturated aqueous NaCl solution. The organic layer was dried over anhydrous Na₂SO₄, and the filtrate was evaporated to give an off-white foamy product (16.18 g, 100%). H NMR and ES-MS confirmed the structure.

D4. Synthesis of N⁶-benzoyl-8-(4.7,10-trioxa-1 -tridecaneamino)-3′-O-t-butyldimethylsilyl-5′-O-(4.4′-dimethoxytrityl)-2′-deoxyadenosine (4): N6-benzoyl-8-bromo-3 ′-O-t-butyldimethylsilyl-5 ′-O-(4,4′-dimethoxytrityl)-2′-deoxyadenosine (Compound 3) (8.31 gm, 9.7 mmol) was dissolved in 200 ml of ethanol then 4,7,10-trioxa-1,13-tridecanediamine (6.75 gm, 6.7 ml, 30 mmol) was added at once and kept stirring at 50° C. After 16 hrs TLC showed that all starting material was converted to one major product with lower Rf and other minor products. The solvent was evaporated to dryness, dissolved in dichloromethane, washed three times with a solution of brine, dried over anhydrous Na₂SO₄, then evaporated to give a yellow gummy material. Column chromatography (1% TEA+CH₂CI₂) to (1% TEA+5% MeOH+CH₂Cl₂) permitted the purification of the major product as an off-white gummy material (4.53gm, 47%). This product was identified with HNMR and ES-MS.

D5. Synthesis of N⁶-benzoyl-8-(4.7,10-trioxa-1-tridecaneaminobiotin)-3′-O-t-butyldimethylsilyl-5′-O-(4,4′-di ethoxytrityl-2′-deoxyadenosine(5): N6-benzoyl-8-(4,7,10-trioxa-1-tridecaneamino)-3′-O-t-butyldimethylsilyl-5′-O-(4,4′-dimethoxytrityl)-2′-deoxyadenosine (Compound 4) (4.53 gm. 4.57 mmol) and biotin-NHS ester (3.12 gm, 9.13 mmol) were dissolved in 75 mL of DMF and few drops of TEA were added and the reaction was stirred at RT. After 2 hrs TLC on silica gel (5% MeOH+1% TEA+94% CH₂Cl₂) showed the formation of one major product less polar than starting material and another minor spot has lower Rf. The solvent was evaporated to dryness, then dissolved in CH₂C1₂ and washed three times with a saturated solution of NaCl, dried the organic layer, evaporated to dryness to leave a yellow gummy material. This material was purified with column chromatography on silica gel by using (1% TEA+CH₂Cl₂) to (1% TEA+2.5% MEOH+CH₂Cl₂) as eluent. After evaporating the fractions containing the product, gave a yellowish solid material (3.16 g, 78%). INMR and ES-MS confirmed the structure.

D6. Synthesis of N6-benzoyl-8-(4.7,1 0-trioxa-1-tridecaneaminobiotin)-5 ′-O-(4,4′-dimethoxytrityl)-2′-deoxyadenosine (6): N⁶-benzoyl-8-(4,7,10-trioxa-1-tridecaneaminobiotin)-3′-O-t-butyldimethylsilyl-5′-O-(4,4′-dimethoxytrityl)-2′-deoxyadenosine (Compound 5) (3.16 gm, 2.6 mmol) was dissolved in 100 mL of dry TKF, and then about (3.25 ml, 3.25 mmol) of tetrabutylammonium fluoride in THF was added in 5 min while stirring at RT. After 8 hrs, TLC on silica gel (10% MeOH+1% TEA+89% CH₂Cl₂) showed that a new product with lower R_{f} formed. The solvent was evaporated to give a yellow oily material. Column chromatography on silica gel eluted with (99% CH₂Cl₂+1% TEA) to (5% MeOH+1% TEA+94% CH₂Cl₂) permitted the purification of the product as a white foamy product (2.86 g, 100%). HNMR and ES-MS confirmed the structure.

D7. Synthesis of N6-benzoyl-8-(4.7.10-trioxa-1-tridecaneaminobiotin)-3′-O-[(diisopropylamine)(2-cyanoethoxy) phosphino)]-5′-O-(4,4′-dimethoxytrityl)-2′-deoxyadenosine (7): N⁶-benzoyl-8-(4,7,10-trioxa-1-tridecaneaminobiotin)-5 ′-O-(4,4′-dimethoxytrityl)-2′-deoxyadenosine (Compound 6) (0.959 gm, 0.86 mmol) was dissolved in a mixture of dry acetonitrile (200 mL) and dichloromethane (50 mL), and diisopropylamine (224 μL, 1.29 mmol) followed by the addition of 2-cyanoethyl N,N, N′, N′-tetraisopropylphosphane (404 μL, 1.29 mmol) and tetrazole (2.6 ml, 1.2 mmol, 0.45 M solution in dry acetonitrile). The addition and subsequent reaction are performed under argon while stirring at RT. After 1.5 h, TLC on silica gel (5% MeOH+5% TEA+45% EA+45% CH₂Cl₂) showed that only about 50% of starting material (SM) was converted to a new product. The same above amount of reagents was added to the reaction and kept stirring for another 2 hrs at RT. TLC showed that about 95% of SM was converted to a new product with higher R_{f}. The solvent was evaporated to dryness then was dissolved in dichloromethane, extracted once with 5% solution of bicarbonate, followed by saturated brine solution and then dried over anhydrous sodium sulfate and evaporated to dryness. Column chromatography on silica gel (10% TEA+45% EA+45% CH₂CI₂) first, then (5% TEA+5% MeOH+45% EA+45% CH₂Cl₂). After evaporating the fractions containing the product, gave a yellow gummy material (774 mg). This material was co-evaporated three times from a mixture of dry benzene and dichloromethane, then was kept in desiccant containing P₂O₅ and NaOH pellets under vacuum for 24 hrs before used in DNA synthesis.

E. Synthesis of Biotinylated 2′-Deoxguanosine Phosphoramidite

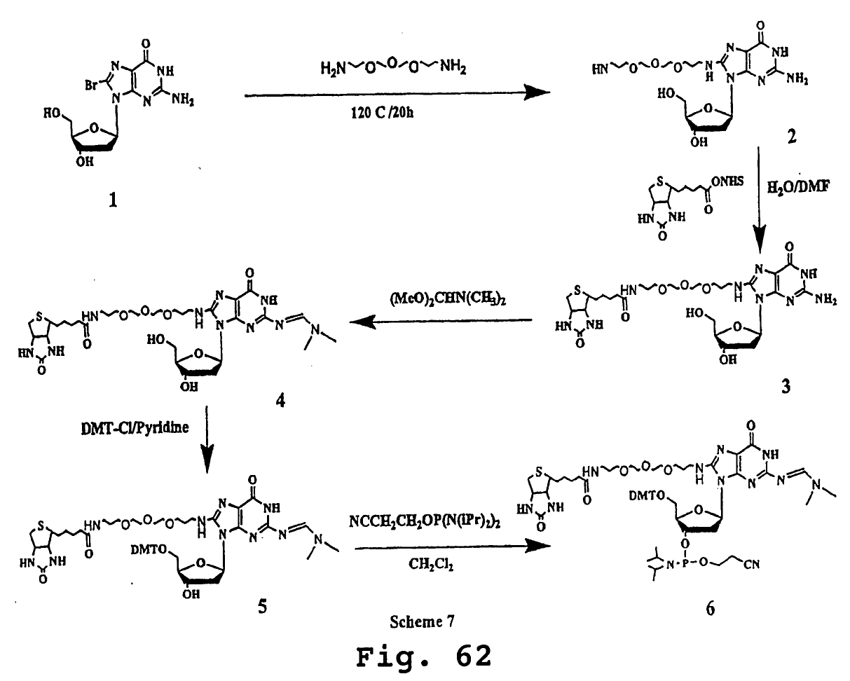

The phosphoramidite of biotin-dG is prepared as set forth in FIG. 62.

E1. Synthesis of 8-(4,7,10-trioxa-1-tridecaneamino)-2′-deoxyguanosine(2): 8-Bromo-2′-Deoxyguanosine (15 g, 43 mmol) and 4,7,10-trioxa-1-tridecanediamine (150 ml) were heated together at 120° C. for 18 h while stirring. TLC on silica gel (20% Methanol (NeOH)+1% triethyl amine (TEA)+79% CH₂Cl₂) showed that all starting material was converted to a very polar product, which gave a positive test with the ninhydrin test. The reaction was cooled down; dichloromethane was added and the mixture was stirred and decanted. This procedure was repeated three times to remove all the diamine. The product, a brown gummy material, was re-dissolved in a mixture of methanol and dichloromethane and precipitated from ether to give a brown gummy product. Flash column chromatography on silica gel (10% MeOH+1% TEA+89% CH₂Cl₂ to 70% MeOH+1% TEA+29% CH₂Cl₂) permitted the purification of the product as a light brown foamy material (18.92 g, 90%). This product was identified with HNMR and ES-MS.

E2. Synthesis of 8-(4,7,10-trioxa-1-tridecaneamidobiotin)-2′-deoxyguanosine (3): Compound (2) (7.72g, 15.89 mmol) was dissolved in 150 ml of water, and then biotin-NHS (8.137 g, 23.83 mmol) was dissolved in DMF and added to the reaction while stirring at RT. After 30 min. TLC on silica gel showed that all starting material was converted to a new product with higher Rf. The reaction was stopped by evaporating all solvent under high vacuum to leave a brown gummy material. Flash column chromatography on silica gel (10% MeOH+1% TEA+89% CH₂Cl₂ to 20% MeOH+1% TEA+79% CH₂Cl₂) permitted the purification of the product as light brown foamy product (10.02 g, 89%). This product was identified with HNMR and ES-MS.

E3. Synthesis ofN²-(N-dimethylformamide)-8-(4,7,10-trioxa-1-tridecaneamidobiotin)-2′-deoxyguanosine (4): Compound (3) (10 g, 14.05 mmol) was dissolved in 300 ml of dry methanol by heating it to about 80° C. and the reaction mixture was allowed to cool down to RT. Then, N,N′-dimethylformamide dimethylacetal (5.97 ml, 44.96 mmol) was added dropwise in 5 min. After the reaction was stirred at RT for 18 h, TLC on silica gel showed that all starting material was converted to a higher Rf product. The reaction was stopped by evaporating all solvent and co-evaporated once from methanol under high vacuum to give a light brown foamy product (10 g, 92%). This product was identified with HNMR and ES-MS.

E4. Synthesis of 5′-O-(4,4′-dimethoxytrityl)-N²-(N,N′-dimethylformamide)-8-(4,7,10-trioxa-1-tridecaneamidobiotin)-2′-deoxyguanosine (5): Compound (4) (7.4 g, 9.65 mmol) was co-evaporated twice under high vacuum from dry pyridine and then was dissolved in dry pyridine (300 ml). DMT-Cl (4.9 g, 14.47 mmol) was added at once and the mixture was kept stirring at RT. After 3h, TLC showed that about 70% of the starting material was converted to a new product. Another 1.3 g of DMT-Cl was added and the mixture was stirred overnight. TLC showed that all starting material was converted to a new product with higher Rf. The reaction was stopped by adding 2 ml of dry methanol and stirring for 15 min. The mixture was evaporated to dryness. This material was co-evaporated twice from a mixture of dichloromethane and toluene and then was purified by flash chromatography on silica gel. After evaporating the fractions containing the product yielded only (2.5 g) of pure product. The remainder of the material appeared to have hydrolyzed to starting material due to the TEA in the column. The product was identified with HNMR and ES-MS.

E5. Synthesis of 3′-O-[(diisopropylamine)(2-cyanoethoxy) phosphino)]-5′-O-(4,4′-dimethoxytrityl-N²-(N,N′-dimethylformamide)-8-(47.710-trioxa-1-tridecaneamidobiotin)-2′-deoxyguanosine (60: Compound (5)(0.962 gm, 0.89 mmol) was dissolved in dry dichloromethane (30 ml) and diisopropylamine (0.186 ml, 1.07 mmol) followed by the addition of 2-cyanoethyl N,N,N′,N′-tetraisopropylphosphane (0.440 ml, 1.33 mmol) and tetrazole (2.2 ml, 0.979 mmol, 0.45 M solution in dry acetonitrile). The addition and subsequent reaction were performed under nitrogen while stirring at RT. After 4 h, TLC on silica gel (10% MEOH+1% TEA+89% CH₂Cl₂) showed that all starting material was converted to a new product with higher Rf The reaction was stopped by the addition of 0.2 ml of dry methanol and the reaction mixture was stirred for 15 min., at RT. The solvent was evaporated to dryness and the residue was dissolved in dichloromethane. The mixture was extracted once with 5% solution of bicarbonate, followed by saturated brine solution and then was dried over anhydrous sodium sulfate and evaporated to dryness. Flash column chromatography on silica gel (5% MeOH+5% TEA+45% ethyl acetate (EA)+45% CH₂Cl₂) first then (7.5% MeOH+5% TEA+42.5% EA+42.5% CH₂Cl₂). Evaporating the fractions containing the pure product gave a foamy light brown product (0.965 g). This material was co-evaporated three times from a mixture of dry pyridine and dry dichloromethane and then was kept in desiccator containing P₂O₅ and NaOH pellets under vacuum for 24 h before used in DNA synthesis.

F. Synthesis of Oligonucleotides Containing Biotin-dC. Biotin-dA and Biotin-dG

The syntheses of oligonucleotides containing biotin-dC, biotin-dA and biotin-dG, site-specifically located, were performed on a CPG support using a fully automated DNA synthesizer and the commercially available fully protected deoxynucleosides phosphoramidites. Syntheses of all these oligonucleotides were carried out at 1.0 and 0.4 μmol scale. The coupling time for the biotin-dC, -dA and -dG were extended to 900 seconds. The coupling efficiency of the biotin-dC, -dA, -dG phosphoramidites was found greater than 96%. After coupling ofthe biotinylated phosphoramidites, the remaining residues comprising the e-tag reporter of interest were added. Upon completion of the synthesis of the oligonucleotides, they were deprotected with concentrated ammonia at 65° C. for 1 hour. These oligonucleotides were purified by reverse-phase “PLC and desalted by OPC column, then used as such.

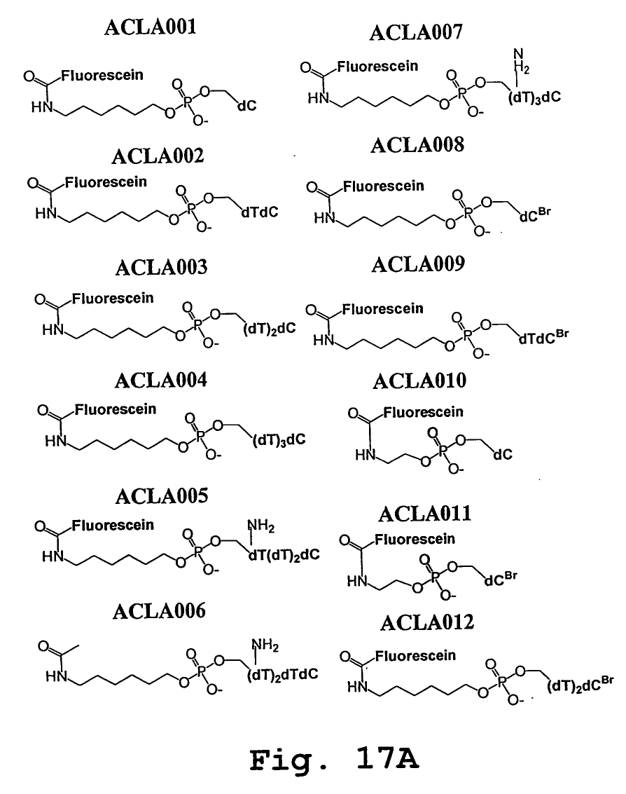

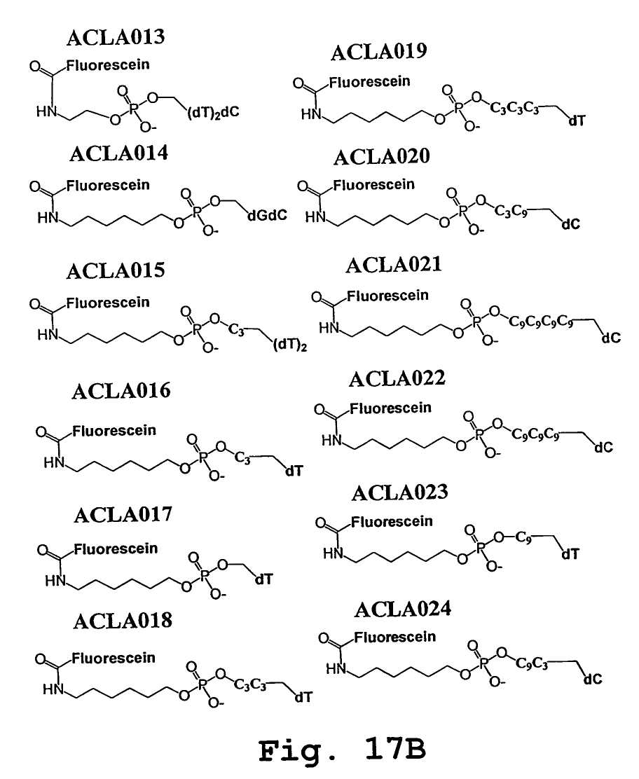

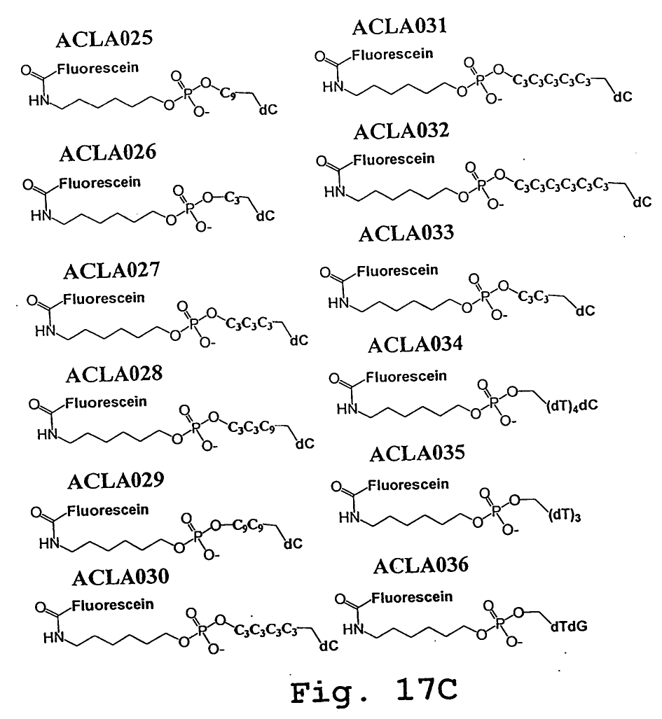

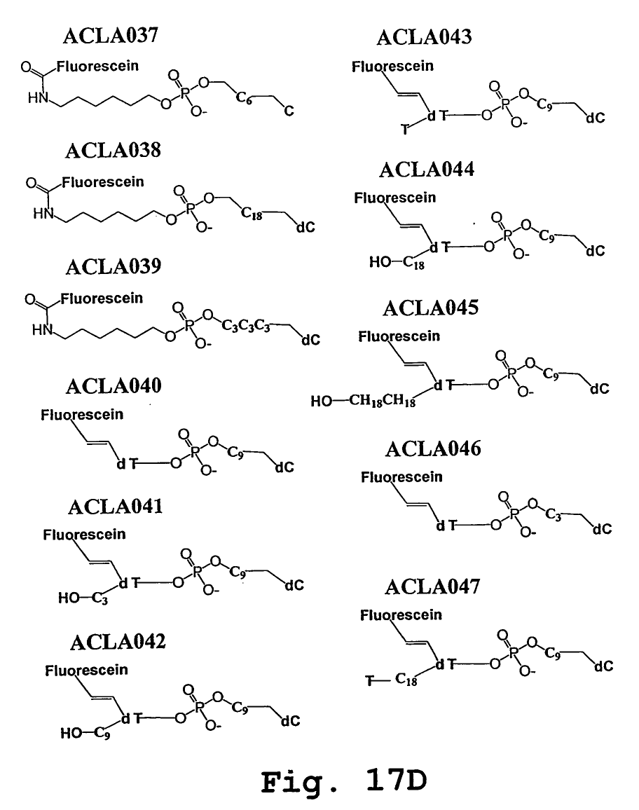

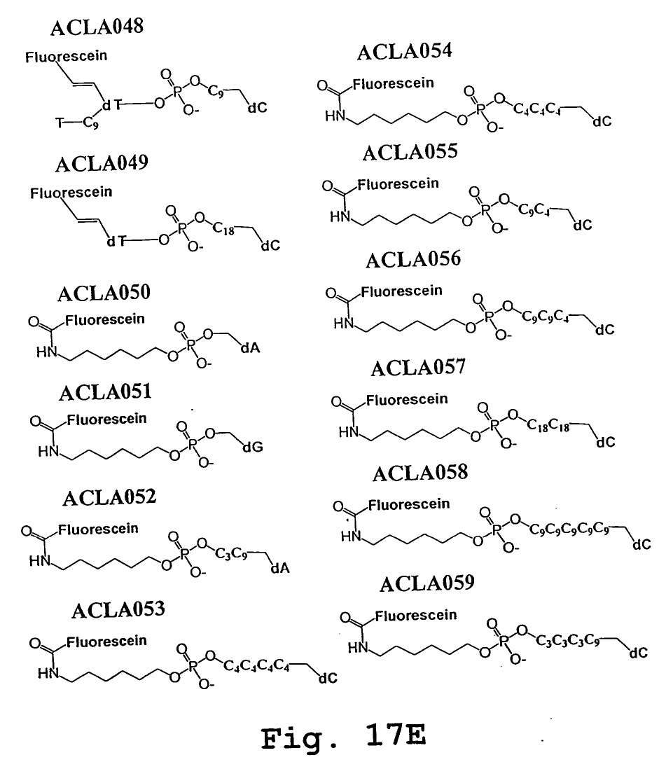

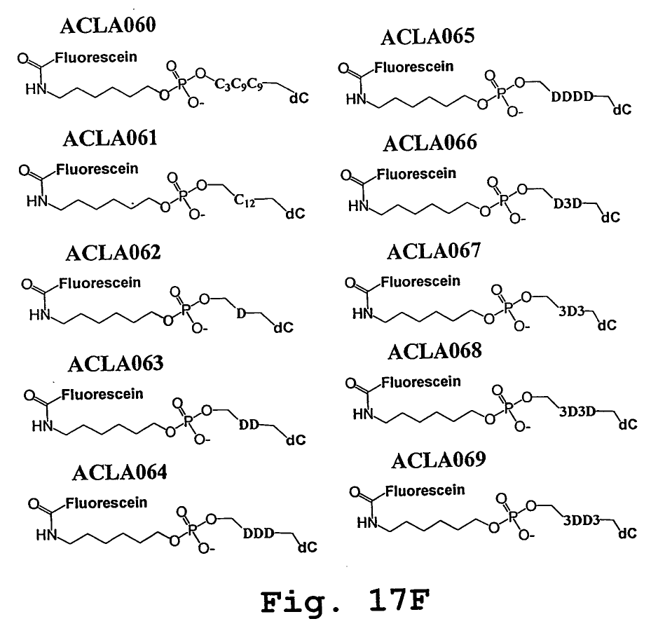

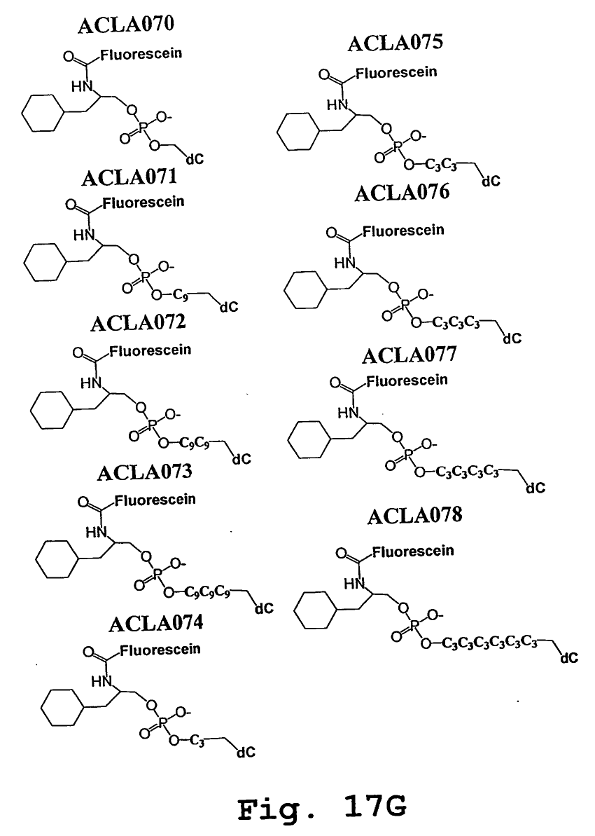

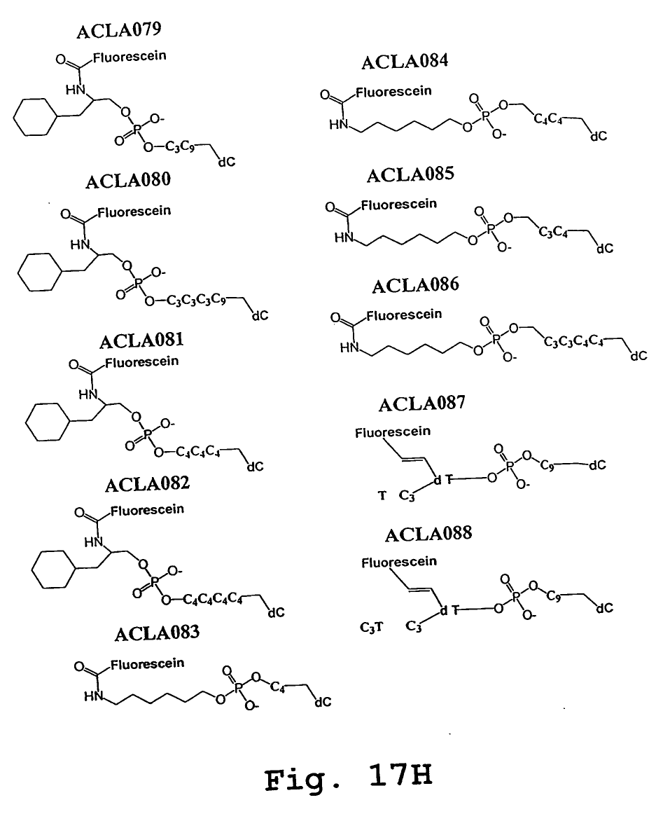

G. Synthesis of ACLAOO1 FIG. 17) on an ABI 394 DNA Synthesizer

6-Carboxyfluorescein (6-FAM) phosphoramidite is prepared by the addition of 2.96ml of anhydrous acetonitrile to a 0.25 gram bottle of the fluorescein phosphoramidite, to give a 0.1 M solution. The bottle is then loaded onto the ABI 394 DNA synthesizer at position 8 using the standard bottle change protocol. The other natural [dA^{bz} (0.1 M: 0.25 g/2.91 mL anhydrous acetonitrile), dC^{Ac}(0.1 M: 0.25g/3.24 mL anhydrous acetonitrile), dT(0.1 M: 0.25 g/3.36 mL anhydrous acetonitrile), dG^{dmf} (0.1 M: 0.25 g/2.81 mL anhydrous acetonitrile)] phosphoramidite monomers are loaded in a similar fashion to ports 14. Acetonitrile is loaded onto side port 18, standard tetrazole activator is loaded onto port 9, CAP A is loaded onto port 11, CAP B is loaded onto port 12, oxidant is loaded onto port 15, and deblock solution is loaded onto port 14 all using standard bottle change protocols.

Standard Reagents Employed for DNA Synthesis:

Oxidizer: 0.02 M Iodine (0.015 M for MGB Probes)

DeBlock: 3% trichloracetic acid in dichloromethane

Activator: 1H-Tetrazole in anhydrous acetonitrile

HPLC Grade Acetonitrile (0.002% water)

Cap A: acetic anhydride

Cap B: N-methyl imidazole

The target sequence of interest is then input with a terminal coupling from port 8 to attach ACLA001 to the 5′-end of the sequence. A modified cycle is then chosen such that the desired scale (0.2 μmol, 1.0 μmol, etc.) of DNA is synthesized. The modified cycle contains an additional wait step of 800 seconds after any addition of 6-FAM. A standard DNA synthesis column containing the support upon which the DNA will be assembled is then loaded onto one of four positions of the DNA synthesizer. DNA containing e-tag reporters have been synthesized on various standard 500 A CPG supports (Pac-dA-CPG, dmf-dG-CPG, Ac-dC-CPG, dT-CPG) as well as specialty supports containing 3′-biotin, 3′-amino linker, and minor grove binding species.

Upon completion of the synthesis, the column is removed from the synthesizer and either dried under vacuum or by blowing air or nitrogen through the column to remove residual acetonitrile. The column is then opened and the CPG is removed and placed in a 1-dram vial. Concentrated ammonia is added (2.0 mL) and the vial is sealed and placed into a heat block set at 65° C. for a minimum of two hours. After two hours the vial is allowed to cool to room temperature after which the ammonia solution is removed using a Pasteur pipette and placed into a 1.5 mL Eppendorf tube. The solution is concentrated in vacuo and submitted for “PLC purification.

H. Synthesis of ACLA002 (FIG. 12 on an ABI 394 DNA Synthesizer

6-Carboxyfluorescein (6-FAM) phosphoramidite is prepared by the addition of 2.96 mL of anhydrous acetonitrile to a 0.25 gram bottle of the fluorescein phosphoramidite, to give a 0.1 M solution. The bottle is then loaded onto the ABI 394 DNA synthesizer at position 8 using the standard bottle change protocol. The other natural [dA^{bz} (0.1 M: 0.25 g/2.91 mL anhydrous acetonitrile), dC^{Ac} (0.1 M: 0.25 g/3.24 mL anhydrous acetonitrile), dT (0.1 M: 0.25 g/3.36 mL anhydrous acetonitrile), dG^{dmf} (0.1 M: 0.25 g/2.81 mL anhydrous acetonitrile)] phosphoramidite monomers are loaded in a similar fashion to ports 1-4. Acetonitrile is loaded onto side port 18, standard tetrazole activator is loaded onto port 9, CAP A is loaded onto port 11, CAP B is loaded onto port 12, oxidant is loaded onto port 15, and deblock solution is loaded onto port 14 all using standard bottle change protocols. The target sequence of interest is then input with a terminal coupling from port 8 and a penultimate coupling of thymidine to the 5′-end of the sequence to assemble ACLA002. A modified cycle is then chosen such that the desired scale (0.2 μmol, 1.0 μmol, etc.) of DNA is synthesized. The modified cycle contains an additional wait step of 800 seconds after any addition of 6-FAM. A standard DNA synthesis column containing the support upon which the DNA will be assembled is then loaded onto one of four positions of the DNA synthesizer. DNA containing e-tag reporters have been synthesized on various standard 500 Å CPG supports (Pac-dA-CPG, dmf-dG-CPG, Ac-dC-CPG, dT-CPG) as well as specialty supports containing 3′-biotin, 3′-amino linker, and minor grove binding species.

Upon completion of the synthesis the column is removed from the synthesizer and either dried under vacuum or by blowing air or nitrogen through the column to remove residual acetonitrile. The column is then opened and the CPG is removed and placed in a 1-dram vial. Concentrated ammonia is added (2.0 mL) and the vial is sealed and placed into a heat block set at 65° C. for a minimum of two hours. After two hours the vial is allowed to cool to room temperature after which the ammonia solution is removed using a Pasteur pipette and placed into a 1.5 mL Eppendorf tube. The solution is concentrated in vacuo and submitted for HPLC purification.

I. Synthesis of ACLA003 (FIG. 17) on an ABI 394 DNA Synthesizer

6-Carboxyfluorescein (6-FAM) phosphoramidite is prepared by the addition of 2.96 mL of anhydrous acetonitrile to a 0.25 gram bottle of the fluorescein phosphoramidite, to give a 0.1 M solution. The bottle is then loaded onto the ABI 394 DNA synthesizer at position 8 using the standard bottle change protocol. The other natural [dbz (0.1 M: 0.25 g/2.91 mL anhydrous acetonitrile), dCAr (0.1 M: 0.25 g/3.24 mL anhydrous acetonitrile), dT (0.1 M: 0.25 g/3.36 mL anhydrous acetonitrile), dGd (0.1 M: 0.25 g/2.81 mL anhydrous acetonitrile)] phosphoramidite monomers are loaded in a similar fashion to ports 1-4. Acetonitrile is loaded onto side port 18, standard tetrazole activator is loaded onto port 9, CAP A is loaded onto port 11, CAP B is loaded onto port 12, oxidant is loaded onto port 15, and deblock solution is loaded onto port 14 all using standard bottle change protocols. The target sequence of interest is then input with a terminal coupling from port 8 and two penultimate couplings of thymidine to the 5′-end of the sequence to assemble ACLA003. A modified cycle is then chosen such that the desired scale (0.2 μmol, 1.0 μmol, etc.) of DNA is synthesized. The modified cycle contains an additional wait step of 800 seconds after any addition of 6-FAM. A standard DNA synthesis column containing the support upon which the DNA will be assembled is then loaded onto one of four positions of the DNA synthesizer. DNA containing e-tag moieites have been synthesized on various standard 500 Å CPG supports (Pac-dA-CPG, dmf-dG-CPG, Ac-dC-CPG, dT-CPG) as well as specialty supports containing 3′-biotin, 3′-amino linker, and minor grove binding species.

Upon completion of the synthesis, the column is removed from the synthesizer and either dried under vacuum or by blowing air or nitrogen through the column to remove residual acetonitrile. The column is then opened and the CPG is removed and placed in a 1-dram vial. Concentrated ammonia is added (2.0 mL) and the vial is sealed and placed into a heat block set at 65° C. for a minimum of two hours. After two hours the vial is allowed to cool to room temperature after which the ammonia solution is removed using a Pasteur pipette and placed into a 1.5 mL Eppendorf tube. The solution is concentrated in vacuo and submitted for HPLC purification.

J. Synthesis of ACLA016 (FIG. 17) on an ABI 394 DNA Synthesizer 6-Carboxyfluorescein (6-FAM) phosphoramidite is prepared by the addition of 2.96 mL of anhydrous acetonitrile to a 0.25 gram bottle of the fluorescein phosphoramidite, to give a 0.1 M solution. The bottle is then loaded onto the ABI 394 DNA synthesizer at position 8 using the standard bottle change protocol. Spacer phosphoramidite C3 (0.25 g) is dissolved in 5.0 mL of anhydrous acetonitrile and loaded onto position 5 of the synthesizer. The other natural [dAbz (0.1 M: 0.25 g/2.91 mL anhydrous acetonitrile), d&C (0.1 M: 0.25 g/3.24 mL anhydrous acetonitrile), dT (0.1 M: 0.25. g/3.36 mL anhydrous acetonitrile), dGW (0.1 M: 0.25 g/2.81 mL anhydrous acetonitrile)] phosphoramidite monomers are loaded in a similar fashion to ports 14. Acetonitrile is loaded onto side port 18, standard tetrazole activator is loaded onto port 9, CAP A is loaded onto port 11, CAP B is loaded onto port 12, oxidant is loaded onto port 15, and deblock solution is loaded onto port 14 all using standard bottle change protocols. The target sequence of interest is then input with a terminal coupling from port 8 and a penultimate coupling of the C3 spacer from port 5 to assemble ACLAO16. A modified cycle is then chosen such that the desired scale (0.2 μmol, 1.0 μmol, etc.) of DNA is synthesized. The modified cycle contains an additional wait step of 800 seconds after any addition of 6-FAM. A standard DNA synthesis column containing the support upon which the DNA will be assembled is then loaded onto one of four positions of the DNA synthesizer. DNA containing e-tag moieties have been synthesized on various standard 500 Å CPG supports (Pac-dA-CPG, dmf-dG-CPG, Ac-dC-CPG, dT-CPG) as well as specialty supports containing 3′-biotin, 3′-amino linker, and minor grove binding species.

Upon completion of the synthesis the column is removed from the synthesizer and either dried under vacuum or by blowing air or nitrogen through the column to remove residual acetonitrile. The column is then opened and the CPG is removed and placed in a 1-dram vial. Concentrated ammonia is added (2.0 mL) and the vial is sealed and placed into a heat block set at 65° C. for a minimum of two hours. After two hours the vial is allowed to cool to room temperature after which the ammonia solution is removed using a Pasteur pipette and placed into a 1.5 mL Eppendorf tube. The solution is concentrated in vacuo and submitted for HPLC purification.

All other e-tag probes are synthesized in a similar manner to that described above.

FIG. 17 provides a list of different e-tag probes with their structures. FIG. 6 provides a list of elution times of some of these e-tag molecules on an ABI 3100 using POP4 as the separation matrix. C₃, C₆, C₉ and C₁₈ are commercially available phosphoramidite spacers from Glen Research, Sterling, Va. The units are derivatives of N,N-diisopropyl, O-cyanoethyl phosphoramidite, which is indicated by Q. The subscripts indicate the number of atoms in the chain, which comprises units of ethyleneoxy terminating in Q with the other terminus protected with DMT. The letters without subscripts A, T, C and G indicate the conventional nucleotides, while _{T}NH₂ intends amino thymidine and C^{Br} intends bromocytidine. In FIG. 8, the numbers indicate the e-tag reporter as indicated in FIG. 17.

Example 2. Synthesis of Catechol PhosPhoramidites

A. Synthesis of Catechol-dA nhosphoramidite (Scheme 3)

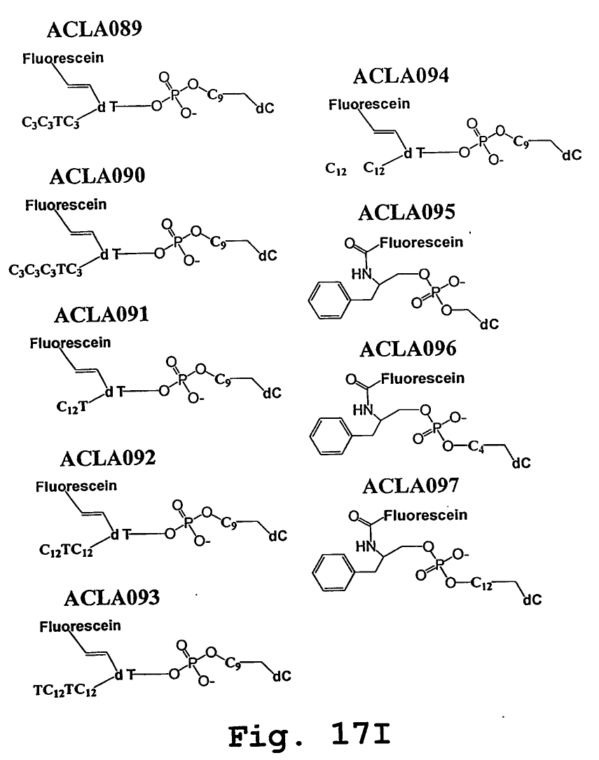

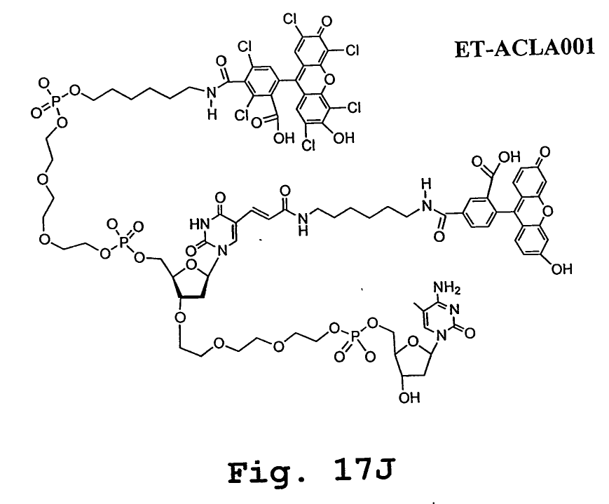

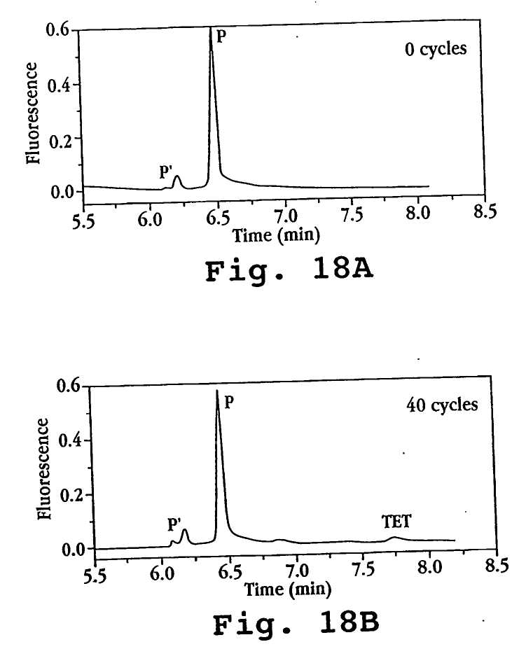

The phosphoramidite of catechol-dA is prepared as set forth in FIG. 18 wherein OAc is acetate, TMS is trimethylsilyl, Bz is benzene, DMT is dimethoxytrityl and Pr is propyl.

A1. Synthesis of 8-Bromo-2′-Deoxyadenosine: 2′-Deoxyadenosine (1) (7g. 25.9 mmol) was dissolved in sodium acetate buffer (150, 1 M, pH 5.0) by warming it to about 50° C. The reaction mixture was cooled down to 30° C. and, then 3 ml of bromine in 100 ml of the same buffer was added dropwise at RT for 15 min. After, 6 hrs. TLC on silica gel (20% MeOH in CH₂Cl₂) showed that all starting material was converted to a new product. The reaction was discolored by adding some sodium metabisulfite (Na₂S₂O₅) while it was stirring, the color changed to a white solution; the pH of the reaction was neutralized by adding NaOH (1M solution). The reaction mixture was kept at 4° C. (refrigerator) for 16 hrs. Next day, the solid material was filtered, washed with cold water and then acetone was added to give a solid yellow powder product (5.75 gm. 64%). The structure of this product was confirmed by ¹H NMR and ES-MS.

A2. Synthesis of N-Benzoyl-8-bromo-5′-0O(4,4′-dimethoxytrityl)-2′-Deoxyadenosine (2Q: 8-Bromo-2′-Deoxyadenosine (7.7 gm. 22.17 mmol) was dried by co-evaporation with dry pyridine and the solid was suspended in 200 ml of dry pyridine followed by the addition of 4,4′-dimethoxytriphenylmethyl chloride (DMT-Cl) (9 gm, 26.6 mmol). After stirring for 4 hrs at RT, TLC on silica gel showed that a new product was formed and some starting material was unreacted. Another amount of DMT-Cl (3 g) was added and stirred at RT for 2 hrs. When TLC showed that all starting material was converted to new product with higher Rf, the reaction mixture was cooled to 0° C. and trimethylchlorosilane (12.042 gm., 14 ml, 110.85 mmol) was added dropwise; and after 40 min., while stirring, benzoyl chloride (15.58 gm, 12.88 ml, 110.85 mmol) was similarly added. The reaction was allowed to react at RT over 2 hrs. The reaction was quenched by slow addition of cold water (50 ml), followed by addition of concentrated ammonia (30%, 50 ml). After 30 min. the reaction mixture was evaporated to dryness. The residue was dissolved in water, and the solution was extracted with ethyl acetate three times, the organic layer washed with saturated sodium bicarbonate solution and then with brine. The organic phase was dried over sodium sulphate and evaporated to dryness. The product was purified by silica column chromatography to give a yellowish solid product (6.79 g, 41.6%). The structure of this product was confirmed by H NMR and ES-MS.

A3. Synthesis of N⁶-benzoyl-8-(4.7.10-trioxa-1-tridecaneamino)-5′-O-(4,4′-dimethoxytrityl-2′-deoxyadenosine (3): N⁶-Benzoyl-8-bromo -5′-O-(4,4′-dimethoxytrityl)-2′-deoxyadenosine (2) (10 gm. 13.57 mmol) was dissolved in 600 ml of dry ethanol then 4,7,10-trioxa-1,13-tridecanediamine (12 ml.) was added at once and stirring was continued at RT. After 16 hrs., TLC on silica gel (5% MeOH+1% TEA+94% CH₂Cl₂) showed that all starting material was converted to one major product with lower Rf and other minor products. The solvent was evaporated to dryness under vacuum without any heat and the residue was re-dissolved in dichloromethane, washed three times with brine, dried over anhydrous Na₂SO₄, and evaporated to give a yellow gummy material. Flash column chromatography on silica gel (1% TEA+CH₂Cl₂) to (1% TEA+5% MeOH+CH₂Cl₂) permitted the purification of the major product as a yellow foamy material (9.43g. 80%). This product was identified with HNMR and ES-MS.

A4. Synthesis of N⁶-benzoyl-8-[13-(N-(3,4-iisobutvrvloxyphenyl acetate) (4,7,10-trioxa-1-tridecaneamino)1-5′-O-(4,4′-dimethoxytrityl)-2′-deoxyadenosine (4): Compound (3) (4 g, 4.56 mmol) was dissolved in 45 ml of dry DMF and N-succinimidyl-(3,4-diisobutyryloxyphenyl) acetate (2.2g, 5.47 mmol) was added after was dissolved in 20 ml of dry DMF. After the reaction was stirred at RT for 30 min, TLC on silica gel (5% methanol+1% TEA+CH₂Cl₂) showed that all starting material was converted to a new product less polar than starting material. The reaction was stopped by evaporating all the solvent to dryness under vacuum. The residue was re-dissolved in dichloromethane, washed five times with brine, dried over Na₂SO₄, filtered, and evaporated to give a yellow gummy material. TLC (2.5% methanol+1% TEA+CH₂Cl₂) showed that the material contained one major product and one minor product. Flash column chromatography on silica gel (1% TEA+99% CH₂Cl₂ to 1% MEOH+1% TEA+98% CH₂Cl₂) permitted the purification of the product. The fractions containing the product were evaporated to give a yellowish foamy product (3.33 g, 62%). IHNMR & MS confirmed the structure of the product.

A5. Synthesis of N⁶-benzoyl-8-(13-(N-(3,4-diisobutyryloxyihenyl)acetate) (4,7,10-trioxa-1-tridecaneamino))-3′-O-[(diisopropylamine)(2-cyanoethoxy) phosphino)]-5′-O-(4,4′-dimethoxytrityl)-2′-deoxyadenosine (5): Compound (4) (0.5 g, 0.424 mmol) was dried by co-evaporation twice from a mixture of dry pyridine and dichloromethane, then was dissolved in 20 ml of dry dichloromethane and cooled down to 0° C. in an ice bath under nitrogen. Diisopropylamine (0.370 ml, 2.12 mmol) was added and the mixture was stirred for 5 min.; and 2′-cyanoethyl N,N-diisopropylchlorophosphoramidite (0.200 ml, 0.88 mmol) was added in 2 min., while the reaction was stirred vigorously. The reaction was stirred for 30 min. at 0° C. TLC on silica gel (5% TEA+47.5% ethyl acetate+47.5% CH₂Cl₂) showed that all starting material was converted to a new product with higher Rf. The reaction was stopped by adding about 0.2 ml of dry methanol and stirring for 10 min. The reaction mixture then was washed three times with brine, dried over Na₂SO₄, filtered, and evaporated to dryness. The product was purified by flash column chromatography on silica gel (5% TEA+47.5% hexane+47.5% ethyl acetate to +5% TEA+47.5% ethyl acetate+47.5% CH₂Cl₂). The fractions containing the product were evaporated to give a yellow gummy product (0.420g, 72%). This material was co-evaporated three times from a mixture of dry dichloromethane and dry benzene under high vacuum and was kept under vacuum for 24 h in a desiccator over P₂O₅ and NaOH pellets before it was used for the synthesis of oligonucleotides.

B. Synthesis of Catechol-dG Phosphoramidite (Scheme 6)

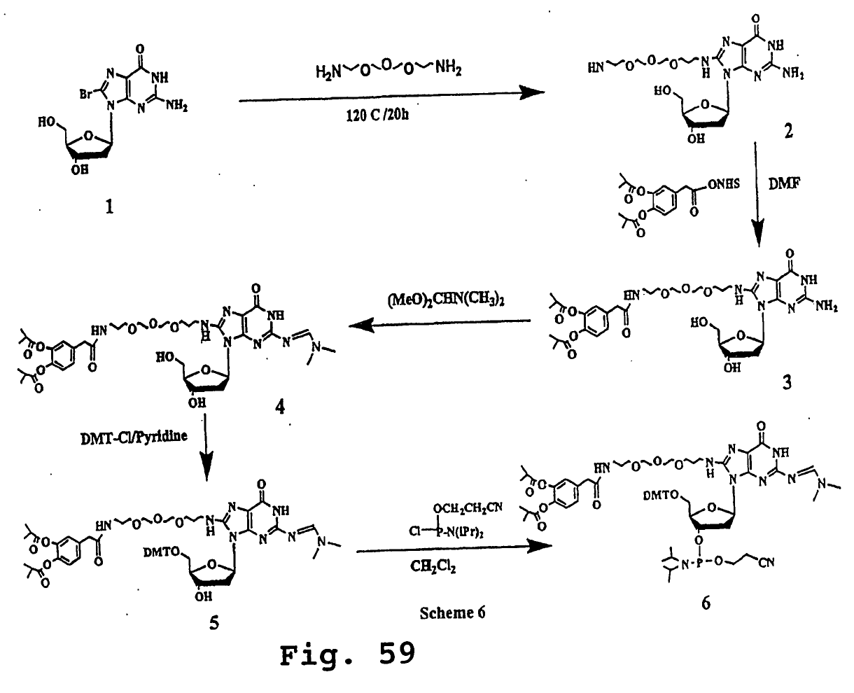

The phosphoramidite of catechol-dG was prepared as set forth in FIG. 59 wherein Pyr is pyridine, MeO is methoxy and iPr is isopropyl.

B1. Synthesis of 8-(4,7,10-trioxa-1-tridecaneamino)-2′-deoxyguanosine (2): 2′-Deoxyguanosine (15 g, 43 mmol) and 4,7,10-trioxa-1-tridecanediamine (150 ml) were heated together at 120° C. for 18 h while stirring. TLC on silica gel (20% MeOH+1% TEA+79% CH₂Cl₂) showed that all starting material was converted to a very polar product, which give a positive test with the ninhydrin test. The reaction was cooled down; dichloromethane was added. The reaction mixture was stirred and decanted. This procedure was repeated three times to remove all the diamine. The product was a brown gummy material and was re-dissolved in a mixture of methanol and dichloromethane and precipitated from ether to give a gummy product. Flash column chromatography on silica gel (10% MeOH+1% TEA+89% CH₂Cl₂ to 70% MEOH+1% TEA+29% CH₂Cl₂) permitted the purification of the product as a light brown foamy material (18.92 g, 90%). This product was identified with HNMR and ES-MS.

B2. Synthesis of 8-13-(N-(3,4-diisobutrrloxyphenyl) acetate) (4,7,10-trioxa-1-tridecaneamido)]-2′-deoxyguanosine (3): Compound (3) was prepared by reacting compound (2) with N-Succinimidyl- (3, 4-diisobutyryloxyphenyl) acetate as in the preparation of catechol-dA (compound 4, scheme 3).

B3. Synthesis of N²-(N,N′-dimethylformamide)-8-[13-(N-(3,4-diisobutyryloxyohenyl) acetate) (4,7,10-trioxa-1-tridecaneamido)]-2′-deoxyguanosine (4): Compound (4) was prepared by reacting compound (3) with N,N′-dimethylformamide dimethylacetal in dry methanol at RT for 18h as described by Sproat et al., Nucleic Acids Research 18, (1990), 41-49.

B4. Synthesis of N²-(N,N′-dimethylformamide)-8-[13-(N-(3,4-diisobutyryloxyphenyl) acetate) (4,7,10-trioxa-1-tridecaneamido)1-5′-O-(4,4′-dimethoxytrityl)-2′-deoxyguanosine (5): Tritylation of compound (4) was achieved by reacting compound (4) with DMT-Cl in dry pyridine as described for catechol-dC (compound 4, scheme 4).

B5. Synthesis of N²-(N,N′-dimethylformamide)-8-[13-(N-(3,4-diisobutyryloxyphenyl) acetate) (4,7,10-trioxa-1-tridecaneamido)]-3′-O-[(diisopropylamine)(2-cyanoethoxy) phosphino)1-5′-O-(4,4′-dimethoxytrityl)-2′-deoxyguanosine (6): Phosphytilation of compound (5) was achieved as described for catechol-dA & catechol-dC.

C. Synthesis of Catechol-dC phosphoramidite (Scheme 4)

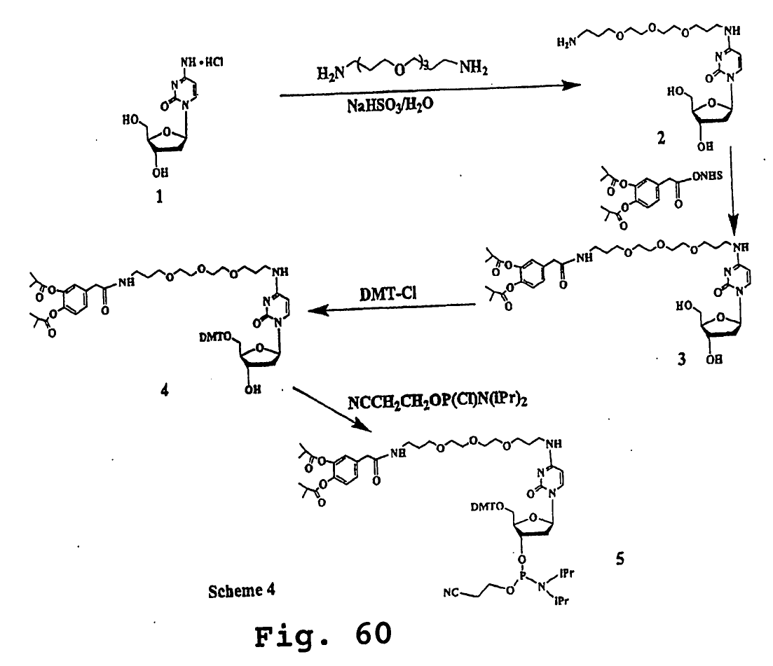

The phosphoramidite of catechol-dC was prepared as set forth in FIG. 60.

C1. Synthesis of N4-(4,7,10-trioxa-1-tridecaneamino)-2′-Deoxycytidine(2): This product was prepared according to a procedure published in J. Am. Chem. Soc. (1989), 111, p6966-6976, with the exception that 4,7,10-trioxa-1,13-tridecanediamine was used instead of 1,3-diaminopropane. ¹HNMR & MS confirmed the structure of the product.

C2. Synthesis of N4-[13-(N-(3,4-diisobutyryloxyphenyl)acetate) (4,7,10-trioxa-1-tridecaneamino)]-2′-Deoxycytidine (3): Compound (2) (2.23g, 5.17 mmol) was dissolved in 25 ml of dry DMF and N-succinimidyl-(3,4-diisobutyryloxyphenyl) acetate (2.5g, 6.2 mmol) was added after was dissolved in 10 ml of dry DMF???. After the reaction was stirred at RT for 2 h, TLC on silica gel (20% methanol in dichloromethane) showed that all starting material was converted to a new product less polar than starting material. The reaction was stopped by evaporating all the solvent to dryness under vacuum. The residue was re-dissolved in dichloromethane, washed five times with water, dried over Na₂SO₄, filtered, and evaporated to give a yellow oily material. Flash column chromatography on silica gel (100% dichloromethane to 10% methanol in dichloromethane). The fractions containing the product were evaporated to give a yellowish foamy product (2g, 54%). 1HN & MS confirmed the structure of the product.

C3. Synthesis of N⁴-[13-(N-(3,4-diisobutyloxyphenyl)acetate) (4,,7,10-trioxa-1-tridecaneamino)]-5′-0-(4.4′-dimethoxytriphenylmethyl )-2′-deoxyctidine(4): Compound (3) (1.82g, 2.52 mmol) was co-evaporated twice from dry pyridine under high vacuum and then was dissolved in 20 ml of dry pyridine; 4,4′-dimethoxytrityl chloride (1.28 g, 3.78 mmol) was added and stirred at RT. After 4 h, TLC on silica gel (10% MeOH+1% TEA+CH₂Cl₂) showed that all staring material was converted to new less polar product. The reaction was stopped by evaporating all pyridine. The residue was re-dissolved in CH₂Cl₂, washed twice with brine, dried over Na₂SO₄, filtered, and evaporated to give a yellow oily material. The product was purified by flash column chromatography on silica gel (1% TEA+CH₂Cl₂ to 1% MeOH+1% TEA+CH₂Cl₂). The fractions. containing the product were evaporated to give an off-white foamy product (2g, 77%). ¹HNMR & MS confirmed the structure of the product.

C4. Synthesis of N⁴-[13-N-(3,4-diisobutyryloxyphenyl) acetate) (4,7,10-trioxa-1-tridecaneaaminol-3′-O-[(diisopropylamine)(2-cyanoethoxyphoshino)]1-5′-0-(4,4′-dimethoxytriphenylmethyl)-2′-deoxycytidine (5): Compound (4) (1 g, 0.97 mmol) was dried by co-evaporation twice from a mixture of dry pyridine and dichloromethane and then was dissolved in 20 ml of dry dichloromethane and cooled down to 0° C. in an ice bath under nitrogen. Diisopropylamine (0.838 mL 4.8 mmol) was added and stirred for 5 min., and 2′-cyanoethyl N,N-diisopropylchlorophosphoramidite (0.325 ml, 1.45 mmol) was added in 5 min. while the reaction mixture was stirred vigorously. The reaction was stirred for 30 min. at 0° C. TLC on silica gel (10% TEA+45% ethyl acetate+45% CH₂Cl₂) showed that all starting material was converted to a new less polar product. The reaction was stopped by adding about 0.2 ml of dry methanol. The reaction mixture was stirred for 5 min. and then was washed three times with brine, dried over Na₂SO₄, filtered, and evaporated to give a yellow oily material. This product was purified by flash column chromatography on silica gel (5% TEA+20% hexane+75% ethyl acetate to +10% TEA+45% ethyl acetate+45% CH₂Cl₂). The fractions containing the product were evaporated to give a yellowish foamy product (0.510 g, 43%). This material was co-evaporated twice from a mixture of dry dichloromethane and benzene under high vacuum. The material was kept under vacuum for 24h in a desiccator over P₂O₅ and NaOH pellets before it was used for the synthesis of oligonucleotides.

D. Synthesis of Catechol-dT Phosphoramidite (Scheme 5)

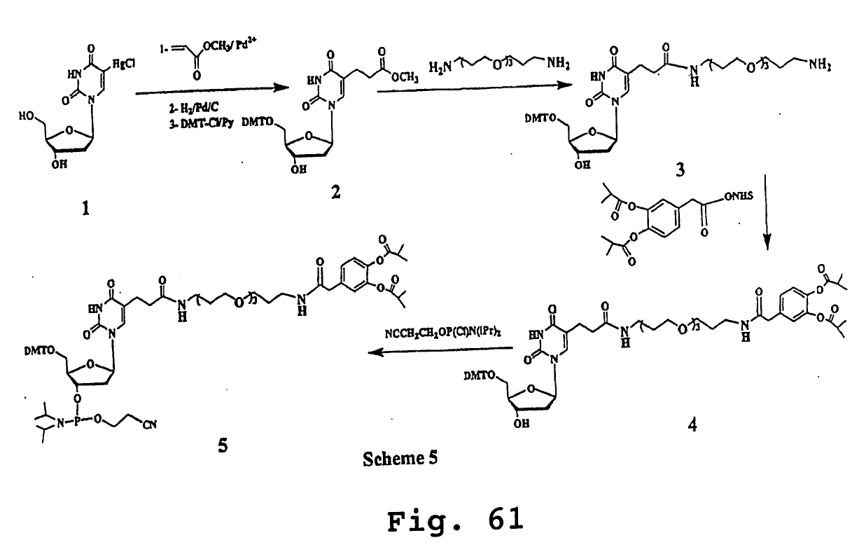

The phosphoramidite of catechol-dT was prepared as set forth in FIG. 61.

D1. Synthesis of 5′-O-(4,4′-dimethoxytrityl)-2′-deoxyuridine-5-(propionic acid methyl ester) (2): This compound was synthesized as described in J. Am. Chem. Soc. (1989), 111, p 6966-6976.

D2. Synthesis of 5′-O-(4,4′-dimethoxytrityl)-2′-deoxyuridine-5-[N-(13-(4,7,10-trioxa-1-tridecaneamino))] propionamide (3): Compound (3) was prepared by reacting compound (2) with 4,7,10-trioxa-1,13-tridecanediamine instead of ethylenediamine, as described by Dreyer et al., Proc. Natl. Acad. Sci. U.S.A. (1985), 82, 968.

D3. Synthesis of 5′-O-(4,4′-dimethoxytrityl)-2′-deoxyrridine-5-[13-(N-(3,4-diisobutyloxyphenyl) acetate) (4.7, 10-trioxa-1-tridecaneamino)]propionamide (4): Compound (4) was prepared by reacting compound (3) with N-Succinimidyl-(3,4-diisobutyryloxyphenyl) acetate in dry DMF as described in the preparation of catechol-dA and catechol-dC.

D4. Synthesis of 3′-O-[(diisopropylamine (2-cyanoethoxy) phosphino)1-5′-O-(4,4′-dimethoxytrityl)-2′-deoxyuridine-5-[13-(N-(3,4-diisobutyryloxyohenyl)acetate) (4,7,10-trioxa-1-tridecaneamino)propionamide (5): Compound (5) was prepared by phosphitylation of compound (4) as described in the preparation of catechol-dA phosphoramidite.

Synthesis of Oligomers Containing Catechol

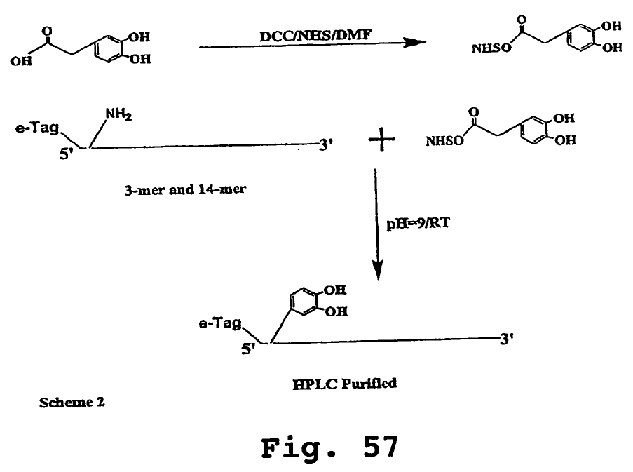

Oligonucleotides containing a catechol moiety were prepared as set forth in FIG. 57. Synthesis of oligonucleotides containing catechol are carried out either by fully automated DNA synthesizer or by post-DNA synthesis.

A—Synthesis of Oligonucleotides Containing Catechol-dA or Catechol-dC on a Solid Support using DNA Synthesizer:

Syntheses of oligonucleotides containing Catechol-dA or Catechol-dC site-specifically located were performed on a CPG support using a fully automated DNA synthesizer and commercially available, protected deoxynucleoside 2-cyanoethyl phosphoramidites for ultramild synthesis. Syntheses of all oligonucleotides were carried out at 0.2 μmol scale. The DNA synthetic cycle consisted essentially of ditritylation, condensation, capping, and oxidation stages. Coupling time was increase to 15 min. when DMT-phosphoramidite of catechol-da 6r catechol-dC incorporated. Coupling efficiency of the modified DMT phosphoramidite of catechol-da or catechol-dC in the syntheses of oligonucleotides was found to be about 98%. Upon completion of the synthesis, all base-labile protecting groups on the oligonucleotides were removed by treatment with concentrated ammonia in the presence of either mercaptoethanol (0.25 M), or 5 mg of ascorbic acid at RT. for 2 h. The oligomer was purified by HPLC on a reverse-phase column, then was desalted and used as such.

B—Synthesis of Oligonucleotides Containing Catechol by Post-Synthesis of DNA (Scheme 2):

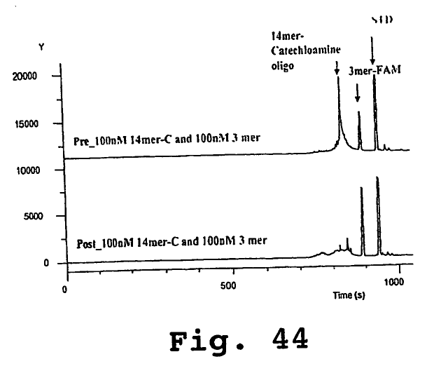

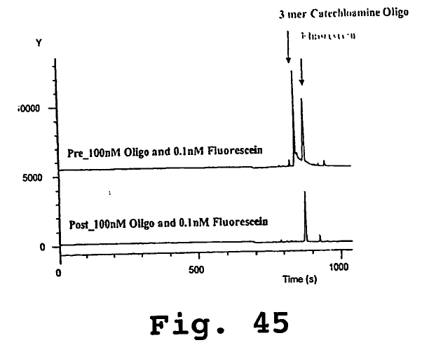

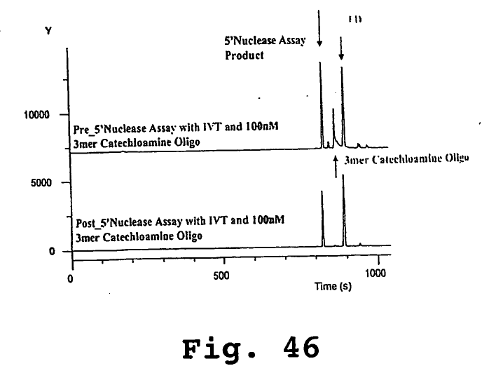

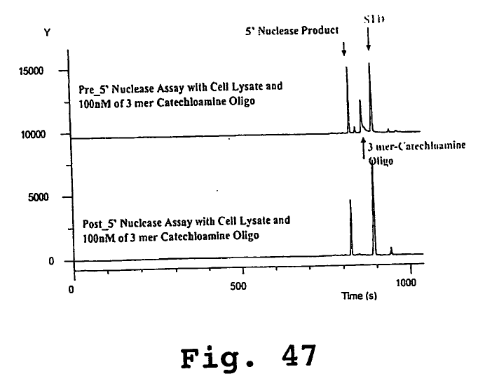

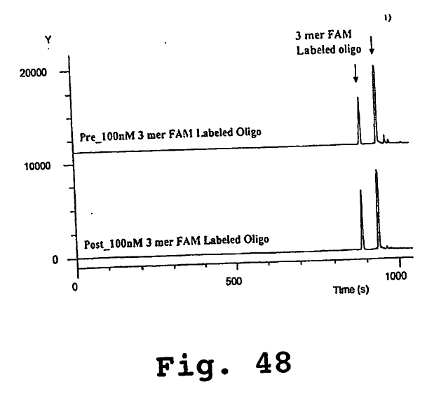

The first step was the synthesis of oligonucleotides, 3-mer and 1 mer, containing an amino group in any desired location followed by purification. These oligomers were reacted individually with 3, 4-dihydroxyphenyl acetic acid-NHS in a buffer at pH 9 overnight at RT. The oligomers were re-purified by BPLC on reverse phase column and desalted and used as such in the experiments shown in the FIGS. 44-48.

### Example 3

### Complexins of Catechol-Oligos with a Boronate Gel

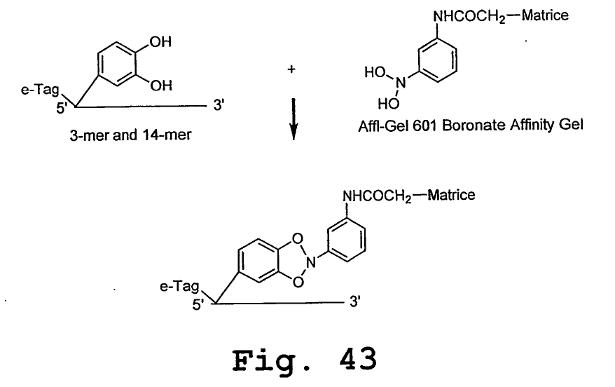

Two oligonucleotides (3 and 14 mers) containing an amino group at the penultimate base were synthesized by standard procedures, purified by HPLC and then reacted with 3,4-dihydroxyphenyl acetic acid-NHS (4 mg) prepared as described above and set forth in FIG. 57, then re-purified with IIPLC. In this manner, a catechol group was introduced into the oligonucleotides. This material was added to a pre-treated Boronate Affinity Gel (Affi-Gel 601, BioRad) at pH 8 to 9 and incubated for 30 min. (see FIG. 43). The supernatant was centrifuiged and was analyzed by capillary electrophoresis using an ABI 3100 machine (Applied BioSystems, Inc., Foster City Calif.). Fluorescein was used as an internal standard. Electropherograms showing the results are depicted in FIGS. 44-48.

### Example 4

### Singleplex Amplifications of Allele 1 and Allele 2

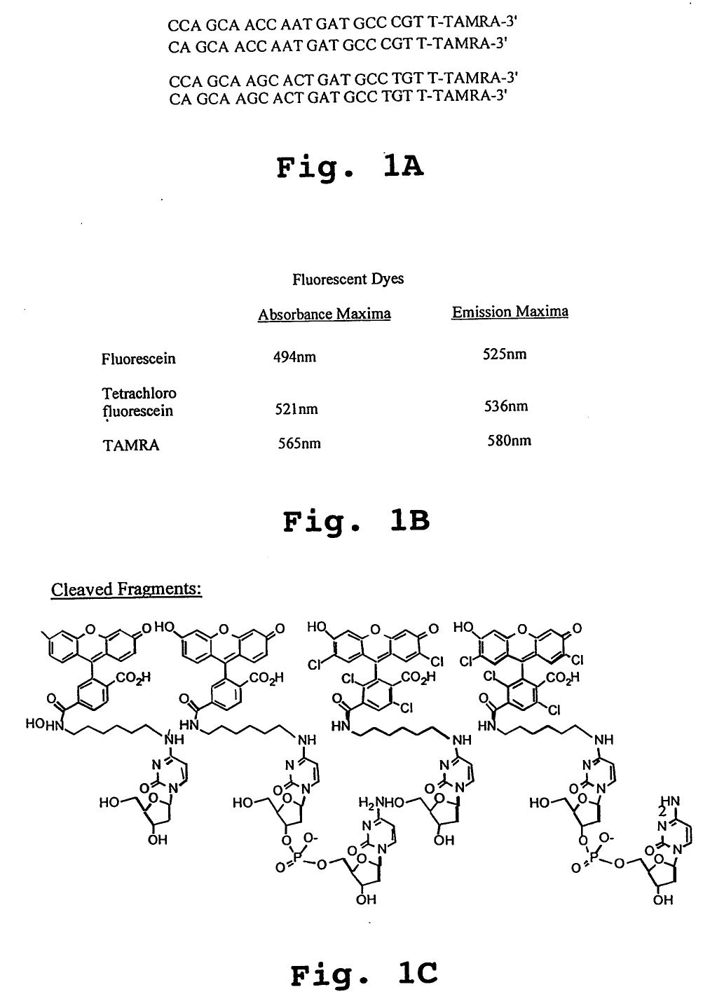

The experiment was set up to run in the following fashion (6 samples, a triplicate for Allele-1 and another triplicate for Allele-2):
22 μL of Mastermix
13 μL of probes and primers (both the probes are present)
4.0 μL of Allele-1 or Allele-2
11 μL of buffer (10 mM Tris-HCl, 1 mM EDTA, pH8.0)
Allele 1 was labeled with tetrachloro fluorescein (TET), and Allele 2 was labeled with fluorescein (FAM), each having characteristics as set forth in FIG. 1B.

The above volumes were added to a PCR tubes and the reaction mixtures were cycled on a Gene Amp® system 9600 thermal cycler (Perkin Elmer) as follows:
50° C.; 2 MIN (for optimal AmpErase UNG activity)
96° C.; 10 MIN (required to activate AmpliTaq Gold DNA Polymerase)
40 cycles of:
95° C.; 15 SEC
60° C.; 60 SEC
70° C.; 10 NM
4° C.; storage

Results from experiments with Allele-1 are shown in FIGS. 18A and B: CE separation of the reaction products of Allele 1 after 0 and 40 cycles. CE instrument was Beckman P/ACE 5000 with LIF detection. BGE: 2.5% LDD30, 7 M urea, 1× TBE. Capillary: 100 μm i.d., 375 μm o.d., Lce=27 cm, Ld=6.9 cm. Detection: λₑₓ=488 nm, λₑₘ=520 nm. Injection: 5 s at 2.0 kV. Field strength: 100 V/cm at room temperature. Peaks: P=unreacted primer; P′=primer product; TET=tetrachlorofluorescell

Results from experiments with Allele-2 are shown in FIGS. 19A and B: CE separation of the reaction products of Allele 2 after 0 and 40 cycles. Experimental conditions were as given above for the FIG. 18 experiment except for the BGE composition: 2.0% LDD30, 1×TBE.

### Example 5A

### Multiplexed Reaction with Allele 1 and Allele 2 in a 1:1 Ratio

The experiment was set up in the following fashion (3 reaction tubes, a triplicate):
22 μL of Mastermix
13 μL of probes and primers (both of the probes were present)
4.0 μL of Allele-1
4.0 μL of Allele-2
7 μL of buffer (10 mM Tris-HCl, 1 mM EDTA, pH 8.0)

The above volumes were added to a PCR tubes and the reaction mixtures were cycled on a Gene Amp® system 9600 thermal cycler (Perkin Elmer) as follows:
50° C.; 2 MIN (for optimal AmpErase UNG activity)
96° C.; 10 MIN (required to activate AmpliTaq Gold DNA Polymerase)
40 cycles of:
95° C.; 15 SEC
60° C.; 60 SEC
70° C.; 10 MIN
4° C.; storage

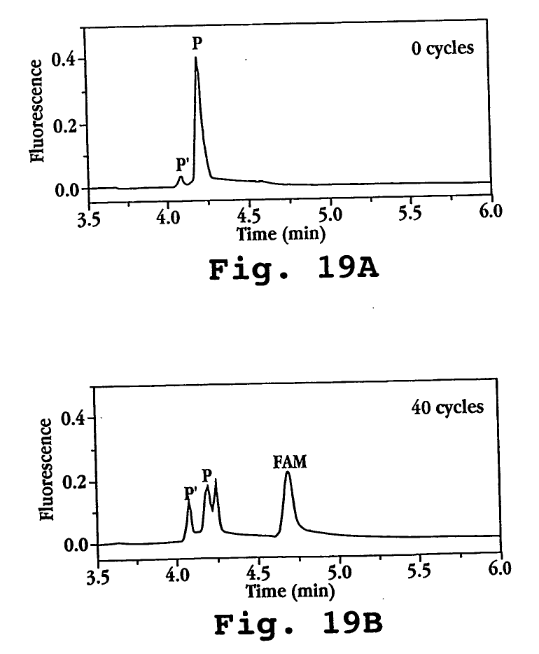

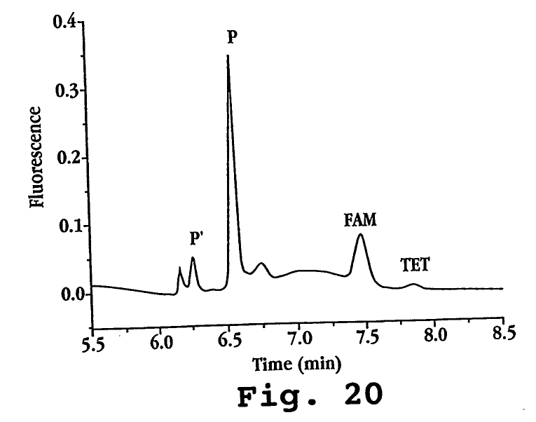

The results are shown in FIG. 20: CE separation of a 1:1 mixture of the 40 cycles products of Alleles 1 and 2. Experimental conditions were as given above for the experiments of FIG. 18.

### Example 5B

### Multiplexed reaction with Allele 1 and Allele 2 in a 10:1 Ratio

The experiment was set up in the following fashion (3 reaction tubes, a triplicate):
22 μL of Mastermix
13 μL of probes and primers (both the probes were present)
5.0 μL of Allele 1
0.5 μL of Allele 2
9.5 μL of buffer (10 mM Tris-HCl, 11 mM EDTA, pH 8.0)

The above volumes were added to a PCR tubes and the reaction mixtures were cycled on a Gene Amp® system 9600 thermal cycler (Perkin Elmer) as follows:
50° C.; 2 M1N (for optimal AmpErase UNG activity)
96 C; 10 MIN (required to activate AmpliTaq Gold DNA Polymerase)
40 cycles of:
95° C.; 15 SEC
60° C.; 60 SEC
70 C; 10 MN
4 C; storage

The results are shown in FIG. 21: CE separation of a 1:10 mixture of the 40 cycles products of Alleles 1 and 2. Experimental conditions were as given for the experiments of FIG. 18.

### Example 6

### Electrophoretic Separation of Reporter Groups on Microfluidic Chip

Label conjugates comprising fluorescein linked to three different peptides, namely, KKAA (SEQ ID NO:5), KKKA (SEQ ID NO:6) and KKKK (SEQ ID NO:7) were prepared as follows: The protected tetrapeptide was prepared on resin using Merrifield reagents. The N-terminus of the last amino acid was reacted with fluorescein N-hydroxysuccinimide (Molecular Probes). The peptides were cleaved from the resin and purified by high performance liquid chromatography (HPLC).

The label conjugates, prepared as described above, and fluorescein were combined in an aqueous buffered solution and were separated and detected in an electrophoresis chip. Detection was 0.5 cm for the injection point on the anodal side of an electrophoresis channel. FITC-KKKK exhibited a negative charge and FITC-KKKA and FITC-KKKK exhibited a positive charge as determined by the migration time relative to EOF. The net charge of FITC-KKKK was greater than +1 and FITC-KKKA and FITC-KKKK migrated electrophoretically against the EOF. The results are shown in FIG. 22.

### Example 7

### Multiplexed Analysis of CFTR SNP Loci with E-Tag Probes

A. Capillary Electrophoresis of CFTR PCR Products with E-Tag Probes on ABI 310

The following example demonstrates separation in a gel based capillary electrophoresis of cleavage of a probe. The conditions employed were: Gel: 2.5% LDD30 in 1×TBE with 7 M urea; CE: PE ABI 310; Capillary: 47 cm long; 36 cm to window; 75 μm ID; Running Buffer: 1×TBE. (LDD30 is a linear copolymer of N,N-diethyl acrylamide and N,Ndimethylacrylamide, 70:30).

The ABI310 was set up in accordance with the directions of the manufacturer. The parameters used were: injection: 5 sec, 2.0 kV; run: 9.4 kV, 45° C., 10 min. To determine the relative mobilities of the digested probes, a spike in system was used. First one digested probe was separated and its peak site determined, then a second probe was spiked into the first probe and the two separated. Then, a third probe was spiked in and separated, and so on until the sites of all six probes was determined. The singleplex PCR runs were first separated followed by separation of the multiplex PCR, which was compared to the S1 digested separation.

B. Multiplexed Amplification of CFTR Fragments with E-Tag Probes

In this study, reactions involved a plurality of probes in the same PCR reaction mixture for different SNPs in the gene for the Cystic Fibrosis transmembrane conductance regulator (CFTR). Taq DNA Polymerase exhibits 5′ to 3′ exonuclease activity, causing degradation of an e-tag probe hybridized to template DNA at the 3′ end of a PCR primer. In the subject example, sequence-specific e-tag probes with a fluorescent dye attached to the 5′ terminus of the probe were employed. PCR was performed with these probes, followed by separation by gel-based capillary electrophoresis to determine cleavage of the e-tag probe. Table 5 indicates the mutation name, exon location, and the nucleotide change and position of the SNP in the CFIR sequence. The name of the oligonucleotide reagents, including e-tag probes and PCR primers, are indicated for each SNP locus. Two PCR primers were generated to amplify each SNP locus, where F indicates the primer in the forward direction, and R indicates the primer in the reverse direction. Two e-tag probes were generated for each SNP locus one hybridizing in the sense direction and one in the antisense direction, indicated as “s” or “as,” respectively. The sequence ID numbers of each of these primers and probes are given in Table 6.

TABLE 5
CFTR SNPs, e-tag Probes, and PCR Primers

| | | | | | Predicted |
|---|---|---|---|---|---|
| Mutation | Exon | Nucleotide | PCR | e-tag | PCR |
| Name | Location | Change | Primers | Probe | Product Size |
| R560T | Exon 11 | G1811C | CF10P (F/R) | CF10s | 108 |
| R560T | Exon 11 | G1811C | CF10P (F/R) | CF10as | 108 |
| D1152H | Exon 18 | G3586C | CF11P (F/R) | CF11s | 188 |
| D1152H | Exon 18 | G3586C | CF11P (F/R) | CF11as | 188 |
| G1349D | Exon 22 | G4178A | CF13P (F/R) | CF13as | 138 |

TABLE 6

Sequence ID Numbers

| Oligonucleotide | SEQ ID NO. |
|---|---|
| CF10P F | SEQ ID NO: 8 |
| CF11P F | SEQ ID NO: 9 |
| CF13P F | SEQ ID NO: 10 |
| CF10P R | SEQ ID NO: 11 |
| CF11P R | SEQ ID NO: 12 |
| CF13P R | SEQ ID NO: 13 |
| CF10s | SEQ ID NO: 14 |
| CF10as | SEQ ID NO: 15 |
| CF11s | SEQ ID NO: 16 |
| CF11as | SEQ ID NO: 17 |
| CF13as | SEQ ID NO: 18 |

The procedure employed in carrying out the singleplex PCR reaction was as follows:

1. Make up Master Mix

| 1x | Component |
|---|---|
| 8 μL | 25 mM MgCl₂ |
| 2.5 μL | 10x PCR Buffer |
| 8 μL | 10 ng/μL DNA template |
| 0.2 μL | 25 mM dNTPs |
| 1 μL | 5 U/μL Taq Gold (added just prior to start of reaction) |

Combine 0.8 μL of 5 μM probe and 1 μL of 10 μM primers to PCR tubes, as indicated below.

| Primer | Probe |
|---|---|
| CF10P | CF10s |
| CF10P | CF10as |
| CF11P | CF11s |
| CF11P | CF11as |
| CF13P | CF13as |

2. Aliquot 20.2 μL of the Master Mix to each tube.

3. In a PE2400 thermalcycler:
96° C.; 10 M
40 cycles of:
95° C.; 10 SEC
55° C.; 30 SEC
65° C.; 1 MIN
70° C.; 10 MIN
4° C.; storage

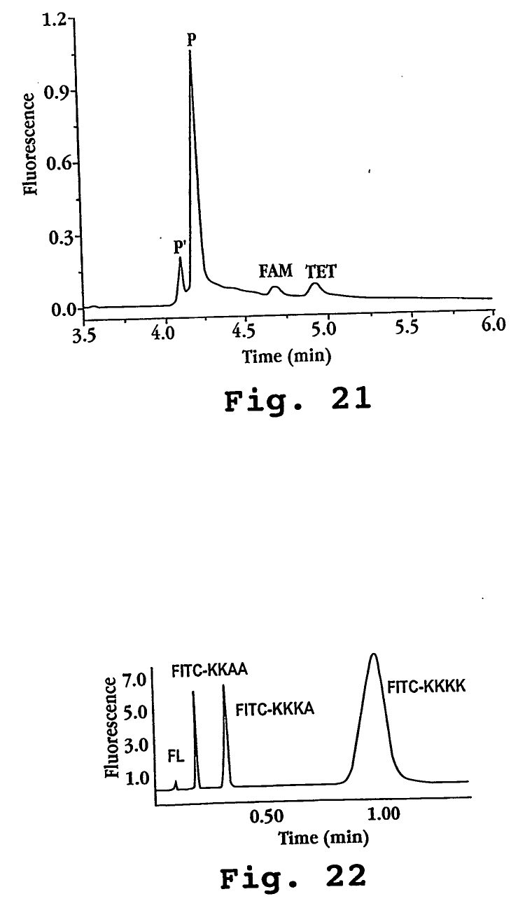

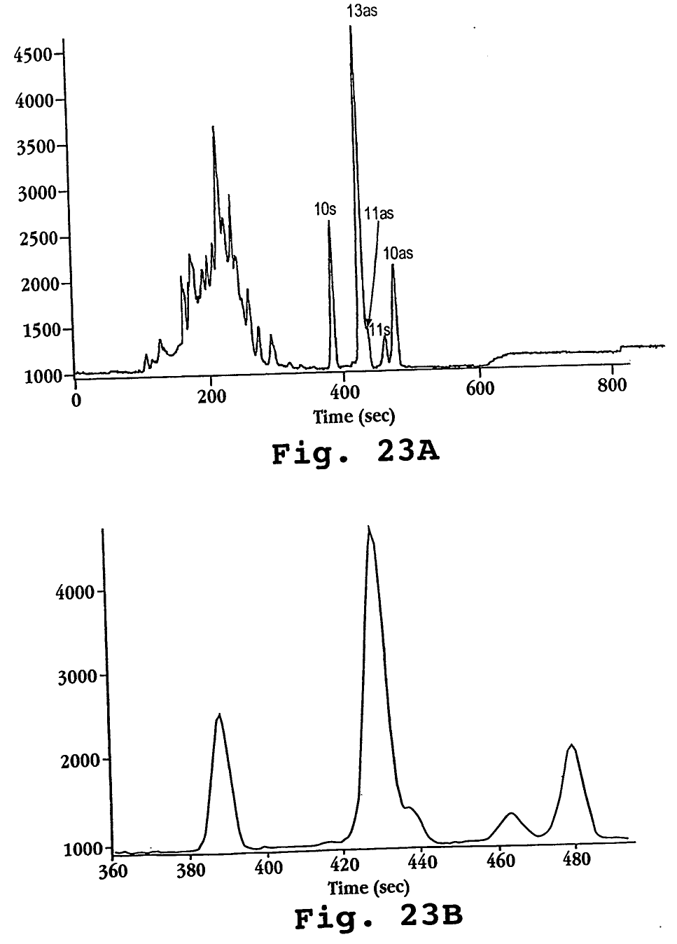

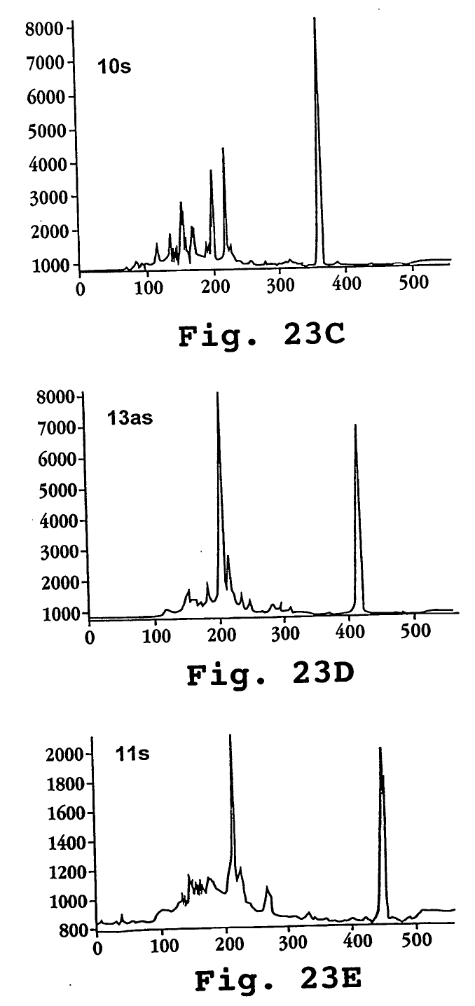

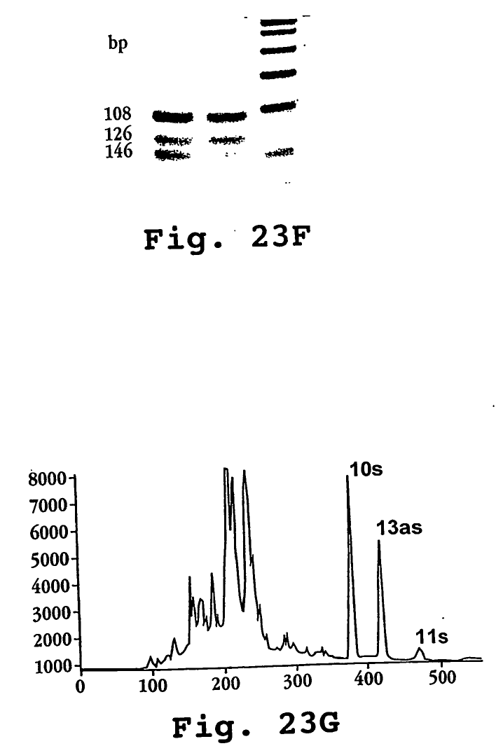

The results are shown in FIG. 23. Results clearly demonstrate formation of a unique electrophoretic tag with a distinct mobility for each amplified sequence. Even in the multiplexed amplification each detection probe gave rise to a unique e-tag reporter with a distinct mobility.

### Example 8

### Electroseparation of Nine E-Tag Reporters on Microfluidic Chip

Label conjugates comprising 9 different fluorescein derivatives linked to thymine: poly deoxythymidine (20-mer; with a 5′ thiol group) is reacted with different maleimide-functionalized fluoresceins after which the product is ethanol precipitated. In a reaction of 12 μL in volume, 10 ∥L of 25 μM oligo, 1 μL 10×S1 nuclease reaction buffer, 1 μL of S1 nuclease incubated at 37° C. for 30 min followed by 96° C. for 25 min. The digested fragments are purified by HPLC.

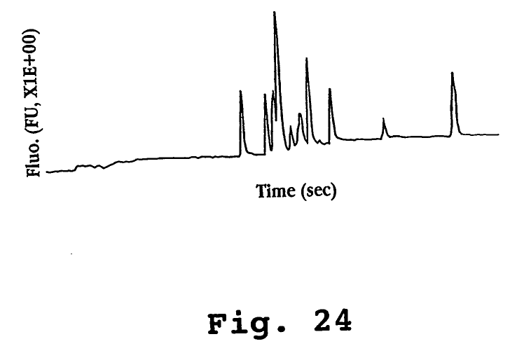

The nine different e-tag reporters prepared as described above and fluorescein were combined in an aqueous buffered and were separated and detected in an electrophoresis chip. Detection was 0.5 cm for the injection point on the anodal side of an electrophoresis channel. The results are shown in FIG. 24.

### Example 9

### Effect of Thiophosphate on 5′-3′ Cleavage

RT-PCR Conditions:

10 μL from a total volume of 25 μL of each mRNA was analyzed in a total volume of 50 μL containing 0.5 μM of each of the oligonucleotide primers, 0.2 mM of each dNTP, 100 nM of each e-tag labeled oligonucleotide probe, 1×RT PCR buffer, 2.5 mM MgCl₂, 0.1U/μL Tfl DNA polymerase and 0.1 U/μL AMV Reverse Transcriptase Promega Access, RT-PCR system).

Reverse Transcription was performed for 45 minutes at 48 IC followed by PCR. (40 thermal cycles of 30 s at 94° C., 1 min at 60° C. and 2 min at 69° C.). MnRNA was obtained from M. Williams, Genentech, Inc. Probe and primer design was performed as described in Analytical Biochemistry, 270, 41-49 (1999). Phosphorothioates were attached to the 2, 3, 4 and 5 phosphate moieties from the 5′ end. Separation was performed as described in the previous section.

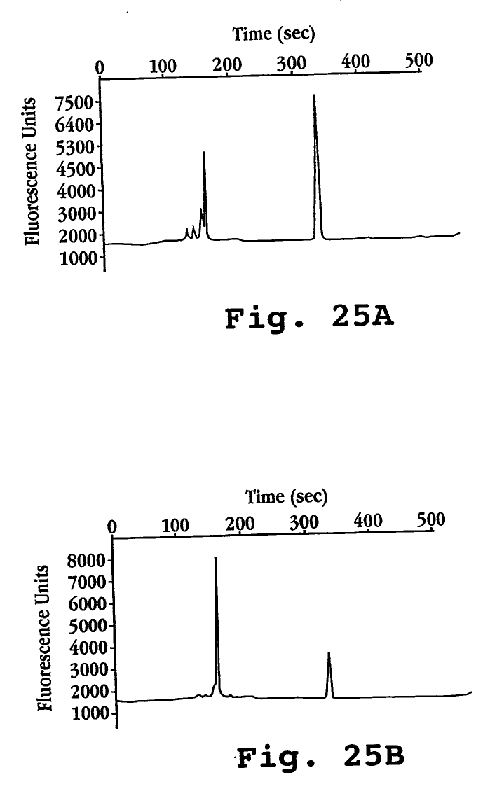

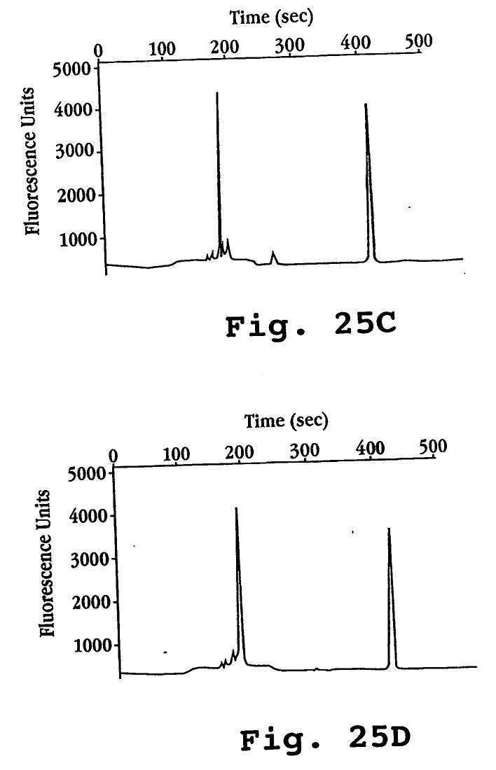

FIG. 25A demonstrates the formation of 5 different cleavage products in the PCR amplification of ANF (anti-nuclear factor) with an e-tag moiety at the 5′ end of the sequence detection probe. In the second experiment, phosphate groups at the 2, 3, 4 and 5 positions are converted into thiophosphate groups. PCR amplification of ANF using a thiophosphate-modified sequence detection probe yielded only one cleavage product (FIG. 25B).

FIG. 25C demonstrates the formation of 3 different cleavage products in the PCR amplification of GAPDH with an e-tag moiety attached to the 5′ end of the sequence detection probe. In a second experiment, phosphate groups at positions 2 and 3 are converted into thiophosphate groups. PCR amplification of GAPDH using the thiophosphate-modified sequence detection probe yielded one predominant cleavage product (FIG. 25D).

The results clearly demonstrate for two different genes that thiophosphate linkages prevent cleavage at multiple sites of a detection probe. A single detectable entity (a single e-tag reporter, FIGS. 25B and D) is generated as a consequence of the amplification reaction.

### Example 10

### S1 Nuclease Digestion of e-tag Probes

In a 1.5 ml tube, 10 μL of e-tag probe was added at a concentration of 10 μM, followed by addition of 1.5 μL of lox S1 nuclease reaction buffer, 0.5 μL of S1 nuclease (Promega, Cat. # M5761, 20-100 unit/μL), and 3 μL of Tris-EDTA buffer to bring the final volume to 15 μL. The reaction was incubated at 37° C. for 20 min followed by 25 min at 96° C. to inactivate the nuclease.

### Example 11

### 5′ Nuclease assays for monitoring specific mRNA expression in cell tysates

THP-1 cells (American Type Culture Collection, Manassas, Va.) were cultured in the presence or absence of 10 nM phorbol 12-myristate 13-acetate (Sigma-Aldrich, St. Louis, Mo.) in RPMI 1640 medium with 10% fetal bovine serum (v/v), 2 mM L-glutamine, 10 mM HEPES, 0.05 mM 2-mercaptoethanol. Twenty-four hours after the induction, cells were harvested and washed twice with PBS before lysed with lysis buffer (20 mM Tris pH 7.5, 0.5% Nonidet P-40, 5 mM MgCl₂, 20 ng/μL tRNA) at 25° C., for 5 min. The lysate was heated at 75° C. for 15 min before testing in a 5′ nuclease assay.

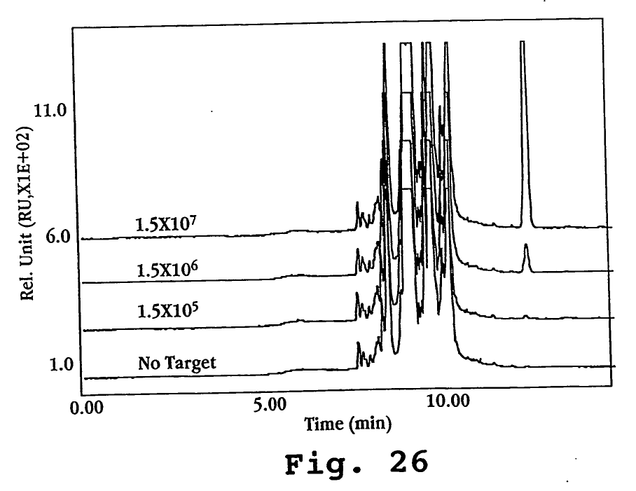

Ten microliters of a cell lysate was combined with a single stranded upstream invader DNA oligo, (5′ CTC-TCA-GTT-CT), a single stranded downstream biotinylated signal DNA oligo (e-tag-labeled), and 2 ng/μL 5′ nuclease (Cleavase IX) in 20 μL of buffer (10 mM MOPS pH 7.5, 0.05% Tween-20 and 0.05% Nonidet P-40, 12.5 mM MgCl₂, 100 μM ATP, 2 U/EL RNase inhibitor). The reactions were carried out at 60° C. for 4 hours before analysis by capillary electrophoresis. To eliminate background signal, due to the non-specific activity of the enzyme, 1 μL of 1 mg/mL avidin was added to the reactions to remove all the e-tag-labeled uncleaved oligo, or e-tag-labeled non-specifically cleaved oligonucleotides. FIGS. 26 and 27, respectively, show separations that were conducted both with and without the addition of avidin.

### Example 12

### PCR Amplification with 5′ Nuclease Activity Using E-Tag Reporters

Exemplary e-tag reporters are shown in FIG. 17. Elution times for some of these reporters on an ABI 3100 using POP4 as the separation matrix are provided in FIG. 6. The e-tag reporters that were prepared were screened to provide 20 candidates that provided sharp separations. 31 e-tag reporters were generated with synthetic targets using the TaqMan (reagents under conditions as shown in the following tabular format. There were 62 reactions with the synthetic targets (one reaction and one negative control for e-tag reporter). Each 25 μL reaction contained 200 nM probe, 500 nM primer, and 5 fM template in 0.5× TaqMan master mix.

All the individual reactions were then run on an ABI 3100 using POP4 as the separation matrix. The samples were diluted 1:20 in 0.5× TaqMan buffer and 1 μL of avidin (10 mg/mL) was added to bind to any intact probe. The sample was further diluted 1:2 with formamide before injecting the sample into the ABI 3100 capillaries. The following are the conditions used with the ABI 3100 for the separation:

| | |
|---|---|
| Temperature | 60° C. |
| Pre-run voltage | 15 kV |
| Pre-run time | 180 sec |
| Matrix | POP4 |
| Injection voltage | 3 kV |
| Injection time | 10 sec |
| Run voltage | 15 kV |
| Run time | 900 sec |
| Run module | e-tag reporter POP4 |
| Dye set | D |

Subsequent separation of multiple e-tag reporters in a single run was accomplished as shown in FIG. 8, the structures of which are identified in FIG. 17.

### Example 13

### E-Tag Reporter Assay for Protein Analysis

A. Labeling of Aminodextran (MW ˜500,000) with E-Tag Reporter and Biotin

Aminodextran was used as a model for demonstrating e-tag reporter release in relation to a high molecular weight molecule, which also serves as a model for proteins. The number of amino groups for 10 mg aminodextran was calculated as 2×10⁻⁸ moles. For a ratio of 1:4 biotin to e-tag reporter, the number of moles of biotin NHS ester employed was 1.85×10, and the number of moles of maleimide NHS ester was 7.4×10⁻⁶. 10.9 mg of aminodextran was dissolved in 6 mL of 0.1% PBS buffer. 10 mg of Biotin-x-x NHS ester and 23.7 mg of EMCS were dissolved together in 1 mL of DMF, and added in 50 μL portions at 30 min intervals to the aminodextran solution while it was stirring and keeping away from the light. After the final addition of the DMF solution, the mixture was kept overnight (while stirring and away from the light). Then, the mixture was dialyzed using a membrane with a molecular weight cut-off of 10,000 Daltons. The membrane was immersed in a beaker containing 2 L of water while stirring. The water was changed four times in a 2 h interval. The membrane was kept in the water overnight (while stirring and keeping away from the light). Then the solution was lyophilized and the lyophilized powder was used for e-tag reporter labeling.

B. Reaction of Biotin and Maleimide Labeled Aminodextran with the E-Tag Reporter, SAMSA.

SAMSA [5-(((2-(and-3)-S-acetylmercapto)succinoyl)amino)fluorescein]was employed as an e-tag reporter to react with maleimide in the aminodextran molecule. For this purpose 0.3 mg (˜5.3×10⁻⁹ moles) of biotin and EMCS labeled with aminodextran were dissolved in 10 μl of water and then reacted with 10 times the mol ratio of SAMSA, for the complete conversion of the maleimide to the e-tag reporter. Therefore, 1.1 mg of SAMSA (1.2×10⁻⁶ moles) is dissolved in 120 μL of 0.1 M NaOH and incubated at room temperature for 15 min (for the activation of the thiol group). Then the excess of NaOH was neutralized by the addition of 2 μL of 6M HCl, and the pH of the solution was adjusted to 7.0 by the addition of 30 μL of phosphate buffer (200 mM, pH 7.0). The activated SAMSA solution was added to the 10 μL solution of the labeled aminodextran and incubated for 1 h. The e-tag reporter labeled aminodextran was purified with gel filtration using Sephadex G-25 (Amersham), and purified samples were collected.

C. The Release of E-Tag Reporter from Labeled Aminodextran

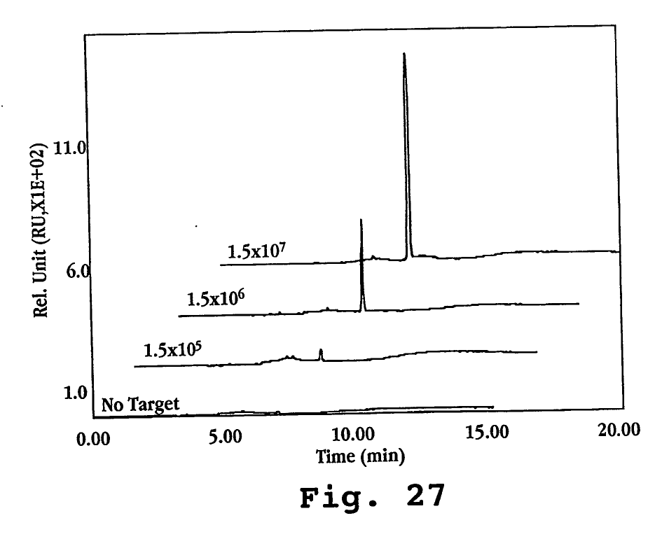

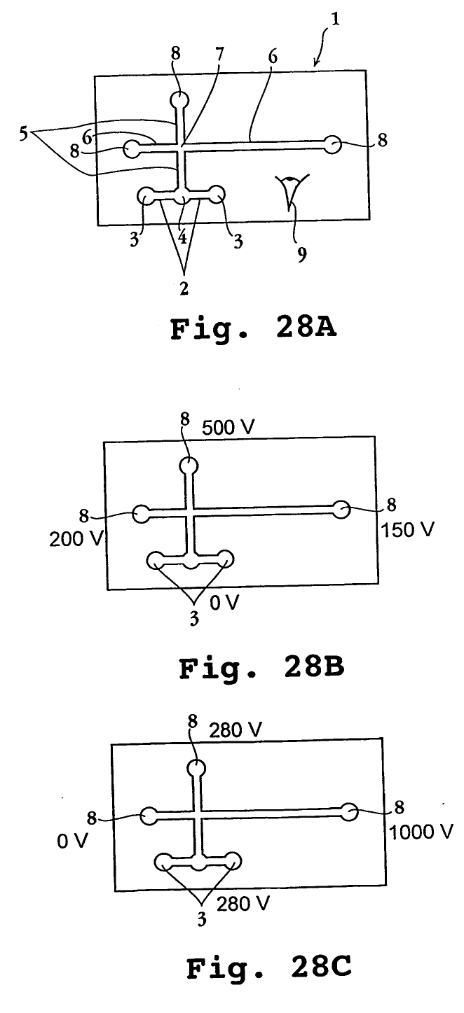

2 μL of streptavidin coated sensitizer beads (100 μg/mL) were added carefully in the dark to the 5 μL of purified labeled aminodextran and incubated in the dark for 15 min. Then the solution was irradiated for 1 min at 680 nm. The release of the e-tag reporter was examined be CE using CE² LabCard™ device. As shown in FIG. 28A, the CE² LabCard™ 1 consists of two parts; evaporation control and injection/separation. The evaporation control incorporates an evaporation control channel 2 (450 μm wide and 50 μm deep) with two replenishment buffer reservoirs 3 (2 mm in diameter) and the evaporation-controlled sample well 4 (1 mm diameter) in the middle of the evaporation control channel. The volume of the replenishment buffer reservoirs are 4.7 μL while the volume of the sample well is only 1.2 μL, and the volume of the channel 2 beneath the middle sample well is about 40 nL. The second part of the CE² device, which is used for injection and separation, consists of an injection microchannel 5 and a separation microchannel 6, intersecting at a junction 7, and having dimensions of 120 μm wide and 50 μm deep. Both ends of the separation channel and one end or the injection channel connect with buffer reservoirs 8, while the second end of the injection channel connects directly to the evaporation-controlled sample well 4. The channels are enclosed by laminating a film (MT40) to the LabCard™. A detector 9 is positioned 10 mm from the junction. After filling the CE² LabCard™ device with separation buffer (20 mM HEPES, pH 7.4 and 0.5% PEO), 300 nL of the assay mixture is added to the sample well 4. The sample was injected into the microchannel junction 7 by applying voltages to the buffer reservoirs as indicated in FIG. 28B. The sample was then separated as is shown in FIG. 28C.

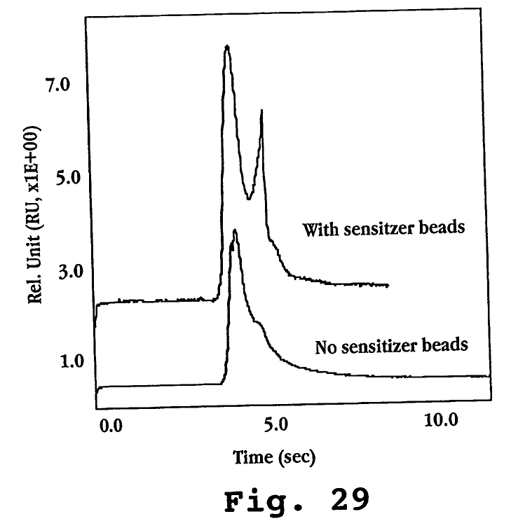

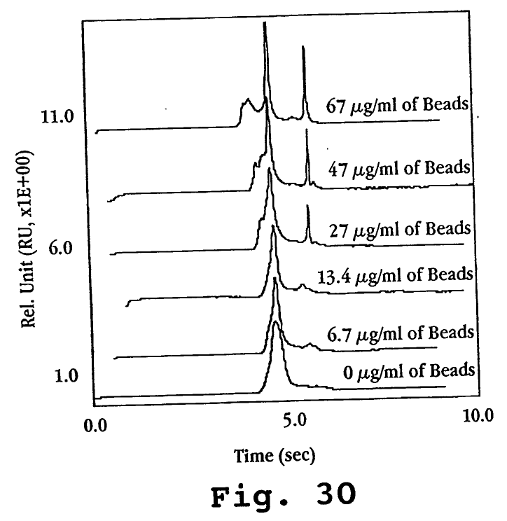

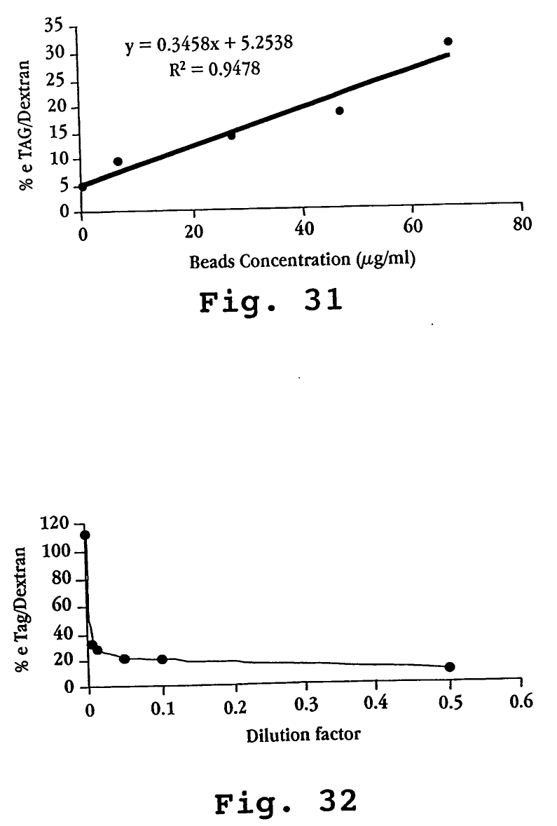

FIG. 29 shows the electropherograms of purified labeled aminodextran with and without sensitizer beads. As shown, the addition of the sensitizer beads leads to the release of the e-tag reporter from the aminodextran using singlet oxygen produced by the sensitizer upon irradiation at 680 nm. In order to optimize the irradiation time, different tubes containing the same mixture of beads and sensitizer were irradiated for different lengths of time ranging from 1 to 10 min. There is no significant increase in the e-tag reporter release for irradiation times longer than 1 min. FIG. 30 shows the effect of sensitizer bead concentration on e-tag reporter release. As depicted in FIG. 30, a higher concentration of sensitizer beads leads to greater release of e-tag reporters from the labeled aminodextran. FIG. 31 depicts a linear calibration curve for the release of e-tag reporters as a function of sensitizer bead concentration. In addition, the effect of the concentration of labeled aminodextran on e-tag reporter release was also examined, with the results shown in FIG. 32. As can be seen, a lower concentration of labeled aminodextran for a given concentration of sensitizer beads leads to more efficient e-tag reporter release (or higher ratio of e-tag reporter released to the amount of labeled aminodextran).

### Example 14

### “Cleanup” of Assay Mixture

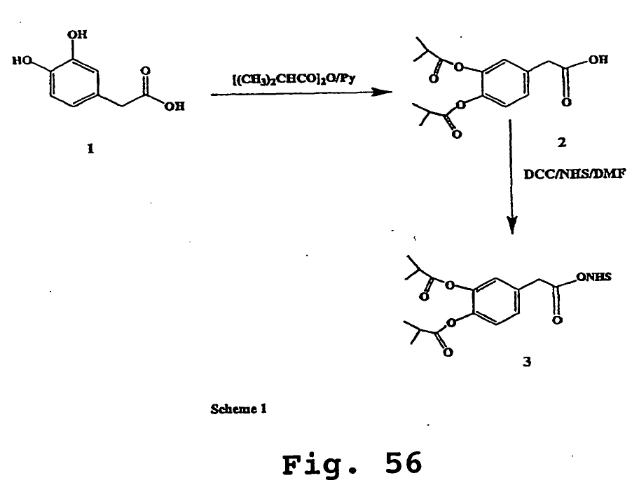

Catechol-NHS is prepared as set forth in FIG. 56 wherein Py is pyridine, DCC is dicyclohexylcarbodiimide, NHS is N-hydroxysuccinimide and DMF is dimethylformamide.

A. Synthesis of 3,4-diisobutrryloxyphenyl acetic acid (2):

3,4-Dihydroxyphenyl acetic acid (1) (5 g, 0.029 mol) was dissolved in dry pyridine (150 ml), then isobutyric anhydride (15 ml, 3 equiv.) was added and stirred at 80° C. for 18 h Thin layer chromatography (TLC) on silica gel (2% Methanol+0.5% Acetic Acid+97.5% Dichloromethane) showed that all starting material was converted to a new less polar product The reaction was stopped by adding 3 ml of water and stirred for about 15 min. at room temperature (RT). Then, the solvent was evaporated under vacuum. The residue was dissolved in dichloromethane, washed three times with brine, dried over Na₂SO₄, filtered, and evaporated under vacuum to give a brown gummy material. This material was purified by flash column chromatography on silica gel (0.5% Acetic Acid+99.5% Dichloromethane) to (1% Methanol+0.5% Acetic Acid+98.5% Dichlioromethane) to give a light brown product as one spot by TLC (8.22 g, 90%). ¹HNMR & MS confirmed the structure.

B. Synthesis of N-Succinimidyl-(3,4-diisobutyryloxyphenyl) acetate (3):

3,4-diisobutyrylphenyl acetic acid (2) (8 g, 0.026 mol), 1,3-dicyclohexylcarbodiimide (DCC)(8.03 g, 0.039 mol, 1.5 equiv.,), and N-hydroxysuccinamide (NHS) (4.48 g, 0.039 mol, 1.5 equiv.,) were dissolved in 100 ml of dimethylformamide (DMF) and stirred for a period of 16 h at RT. TLC on silica gel (2.5% Methanol+97.5% Dichloromethane) showed that all starting material was converted to a new less polar product. The reaction was stopped by filtering off all DCU byproduct and the solvent was evaporated to dryness. The residue was dissolved in dichloromethane, filtered again, and evaporated to give a light brown material. Column chromatography on silica gel (100% dichloromethane to 1% methanol in dichloromethane), permitted the separation of the product as a yellowish solid material (9.2 g, 880%). ¹HNMR & MS confirmed the structure.

It is evident from the above results that the subject inventions provide powerful ways of performing multiplexed determinations. The methods provide for homogeneous and heterogeneous protocols, both with nucleic acids and proteins, as exemplary of other classes of compounds. In the nucleic acid determinations, SNP determinations are greatly simplified, such that the protocol can be performed in only one to four vessels and a large number of SNPs readily determined within a short period of time with great efficiency and accuracy. Genomes can be investigated from both prokaryotes and eukaryotes, including, for prokaryotes, determination of drug resistance, species, strain, etc., and for eukaryotes, determination of species, cell type, response to external stimuli, e.g. drugs or physical changes in environment, mutations, chiasmas, etc. With proteins, one can determine the response of the host cell, organelles or the like to changes in the chemical and physical environments, changes in the surface protein population, changes due to aging, neoplasia, activation, or other naturally occurring phenomenon, where the amount of protein can be quantitated.

Particularly as to nucleic acid determinations, the subject e-tag reporters can be synthesized conveniently along with the synthesis of the oligonucleotides used as probes, primers, etc., where the e-tag reporter is released in the presence of the homologous target sequence. Kits of building blocks or e-tag reporters are provided for use in the different determinations.

It is further evident from the above results that the subject invention provides an accurate, efficient and sensitive process, as well as compositions for use in the process, to perform multiplexed reactions. The protocols provide for great flexibility in the manner in which determinations are carried out and maybe applied to a wide variety of situations involving haptens, antigens, nucleic acids, cells, etc., where one may simultaneously perform a number of determinations on a single or plurality of samples and interrogate the samples for a plurality of events. The events may vary from differences in nucleic acid sequence to proteomics to enzyme activities. The results of the determination are readily read in a simple manner using electrophoresis or mass spectrometry. Systems are provided where the entire process, after addition of the sample and reagents, may be performed under the control of a data processor with the results automatically recorded.

1. A method for isolating a signal produced by a detectable moiety of a released predetermined portion of a probe from signal obtained from intact probe and from other fragments thereof, the method comprising:
(a) combining a probe which comprises a first predetermined portion which is releasable and includes a detectable moiety, and a second portion which includes at least one interactive functionality, with a reagent that potentiates the release of the first portion from the probe;
(b) subjecting the resulting mixture to conditions under which the probe is cleaved, such that the first portion is released from the probe;
(c) contacting the mixture with a capture reagent which binds the interactive functionality, and
(d) separating the capture agent or the released predetermined first portion from the mixture.

2. The method of claim 1, wherein the interactive functionality is a ligand or a chelating moiety.

3. The method of claim 2, wherein the interactive functionality is a chelating moiety, and the capture agent comprises boron, arsenic, a transition metal, or a ferrocene.

4. The method of claim 1, wherein the interactive functionality comprises boron, arsenic, a transitions metal, or a ferrocene, and the capture agent comprises a chelating moiety.

5. The method of claim 2, wherein the interactive functionality is a ligand selected from the group consisting of a small molecule, a receptor for a small molecule, an antigen, an antibody, and an oligonucleotide.

6. The method of claim 1, wherein the probe comprises an oligonucleotide to which the releasable first portion is attached at a nucleotide thereof, and an organic moiety comprising the interactive functionality is attached at a nucleotide adjacent to the nucleotide to which the releasable first portion is attached.

7. The method of claim 6, wherein the nucleotide to which the releasable first portion is attached is a terminal nucleotide.

8. The method of claim 1, wherein the releasable first portion has a predetermined mass or mobility.

9. The method of claim 1, wherein the probe comprises a structure (D, Mⱼ)-L, where
(i) D is a detection group comprising a detectable label;
(ii) L is a cleavable linkage, and
(iii) Mⱼ is a mobility modifier having a mass or charge/mass ratio that imparts a unique and known electrophoretic mobility to a corresponding reporter of the form (D,Mⱼ)-L′, within a selected range of mobilities, with respect to other reporters of the same form in a set of such probes, where L′ is the residue of L following cleavage.

10. A method for detecting binding of or interaction between a target binding agent and any of a plurality of probes, comprising:
(a) subjecting a mixture comprising the target binding agent and the probes to conditions for interaction therebetween, wherein each probe comprises (i) a cleavable reporter group, comprising a detectable moiety and having a mobility, when cleaved, that is unique to the probe among the plurality of probes, and (ii) at least one interactive functionality adjacent the cleavable reporter group;
(b) subjecting the mixture to conditions under which the cleavable moiety is cleaved to release the reporter groups, wherein said cleavage is dependent on said interaction between the target binding agent and any of the plurality of probes;
and wherein the interactive functionality enables the isolation of signal produced by the detectable moieties of the released reporter groups from signal obtained from intact probes and from other fragments thereof,
(c) separating the released reporter groups by their differences in mobility; and
(d) detecting the detectable moieties of released reporter groups, to determine binding of or interaction between the target binding agent and each probe.

11. The method of claim 10, wherein the interactive functionality is effective to quench signal produced by the detectable moiety in intact probe but not in released reporter group.

12. The method of claim 10, wherein said isolation is effected by
contacting the mixture of (b) with a capture reagent which binds the interactive functionality, and
separating the capture agent or the released reporter groups from the mixture.

13. The method of claim 12, wherein the interactive functionality is a ligand or a chelating moiety.

14. The method of claim 12, wherein the interactive functionality is a chelating moiety, and the capture agent comprises boron, arsenic, a transition metal, or a ferrocene.

15. The method of claim 12, wherein the interactive functionality comprises boron, arsenic, a transition metal, or a ferrocene, and the capture agent comprises a chelating moiety.

16. The method of claim 13, wherein the interactive functionality is a ligand selected from the group consisting of a small molecule, a receptor for a small molecule, an antigen, an antibody, and an oligonucleotide.

17. The method of claim 10, wherein each probe comprises an oligonucleotide to which the cleavable reporter group is attached at a nucleotide thereof, and an organic moiety comprising the interactive functionality is attached at a nucleotide adjacent to the nucleotide to which the reporter group is attached.

18. The method of claim 17, wherein the nucleotide to which the reporter group is attached is a terminal nucleotide.

19. A kit for use in detecting the presence and/or amount of each of a plurality of target molecules, comprising in packaged combination:
a plurality of probes, each capable of target-specific binding to a binding site on a selected target molecule, and each having (i) a unique cleavable reporter group, having a detectable reporter moiety and a unique electrophoretic mobility which allows the reporter group to be uniquely identified among all other reporter groups associated with the plurality of probes, and (ii) at least one interactive functionality adjacent the reporter group, wherein the interactive functionality is effective in isolating the signal produced by released reporter groups from the signal produced by intact probes and other fragments thereof.

20. The kit of claim 19, further comprising a capture reagent effective to bind to the interactive functionality.

21. The kit of claim 20, wherein the interactive functionality is a chelating moiety, and the capture reagent comprises boron, arsenic, a transition metal, or a ferrocene.

22. The kit of claim 19, wherein each probe comprises an oligonucleotide to which the cleavable reporter group is attached at a nucleotide thereof, and an organic moiety comprising an interactive functionality attached at a nucleotide adjacent to the nucleotide to which the cleavable reporter group is attached.

23. The kit of claim 22, wherein the nucleotide to which the cleavable reporter group is attached is a terminal nucleotide.

24. A synthetic biopolymer or nucleoside having conjugated thereto a moiety having the formula Ya: 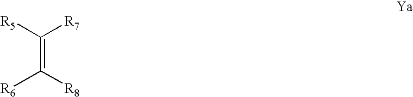
wherein
R⁵ and R⁶ are independently selected from hydroxy, amino, substituted amino, carbonyl, carboxamide and N-hydroxy amide, any of which may be bound to a protecting group, and
R⁷ and R⁸ are independently carboxy or cyano or together form one or more rings, wherein each ring comprises 5 to 10 atoms and one or more double bonds, in addition to the double bond of the above formula, wherein the atoms of each ring are optionally substituted with alkyl, hydroxy, nitro, or oxo.

25. The synthetic biopolymer or nucleoside of claim 24, wherein R⁷ and R⁸ together form a benzene or anthracene ring.

26. The synthetic biopolymer or nucleoside of claim 24, wherein said moiety has the formula Yb: 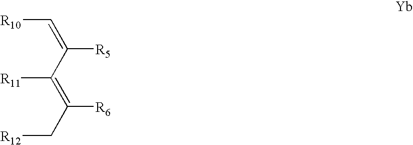
wherein
R⁵ and R⁶ are independently selected from hydroxy, amino, substituted amino, carbonyl, carboxamide or N-hydroxy amide, any of which may be bound to a protecting group, and
R¹⁰ and R¹¹ and R¹² are independently carboxyl or cyano, or together form one or more rings, wherein each ring comprises 5 to 10 atoms and one or more double bonds, in addition to the double bonds of the above formula, wherein the atoms of each ring are optionally substituted with alkyl, hydroxy, nitro or oxo.

27. The synthetic biopolymer or nucleoside of claim 26, wherein R¹⁰ and R¹¹ and R¹² together form a naphthalene ring.

28. The synthetic biopolymer or nucleoside of claim 24, wherein said moiety has the formula Y′a: 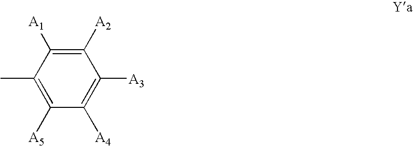
wherein
A¹ to A⁵ are independently selected from hydrogen, hydroxy, amino, substituted amino, carbonyl, carboxamide and N-hydroxy amide, any of which may be bound to a protecting group, wherein at least two adjacent members of A¹ to A⁵ are not hydrogen.

29. The synthetic biopolymer or nucleoside of claim 24, wherein said moiety has the formula Y′b: 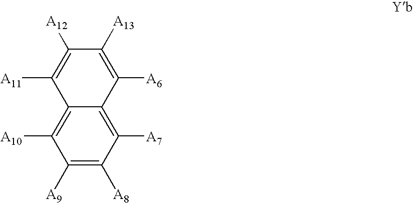
wherein
A⁶ and A⁷ are independently selected from hydroxy, amino, substituted amino, carbonyl, carboxamide and N-hydroxy amide, any of which may be bound to a protecting group,
one of A⁸ to A¹³ is linked to said synthetic biopolymer or biopolymer precursor by a bond or a lining group, and the remaining members of A⁸ to A¹³ are independently hydrogen, hydroxy, amino, substituted amino, carbonyl, carboxamide or N-hydroxy amide.

30. The synthetic biopolymer or nucleoside of claim 24, wherein said moiety comprises a 1,2-diol, 1,3-diol, 1,2-aminoalcohol, 1,3-aminoalcohol, 1,2-hydroxy acid, 1,3-hydroxy acid, 1,2-hydroxy acid amide, 1,3-hydroxy acid amide or dioxime.

31. The synthetic biopolymer or nucleoside of claim 24, wherein said moiety is selected from the group consisting of ortho-hydroxybenzenes (catechols), ortho-hydroxybenzohydroxamic acids, ortho-hydroxybenzoic acids, ortho-hydroxybenzamides, imidazolyl phenols, dihydroxyfumaric acids, hydroxy pyridine aldehydes and hydroxyanthraquinone dioximes.

32. The synthetic biopolymer or nucleoside of claim 24, wherein said moiety is selected from the group consisting of catechol, salicylamide, N-phenylsalicylamidine, 2-(4′imidazolyl)phenol, 1,8-dihydroxynaphthalene, dihydroxyfumaric acid, salicylaldehyde, 3-hydroxypyridine-4-aldehyde, pyridoxamine, 1-hydroxyanthraquinone dioxime and disalicylimide.

33. The synthetic biopolymer or nucleoside of claim 24, wherein the synthetic biopolymer is a polynucleotide or polypeptide.

34. A compound of the formula: 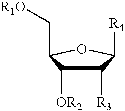
wherein:
R¹ is H, dimethoxytrityl (DMr), triphosphate ester, diphosphate ester, or monophosphate ester;
R² is H or phosphoramidite;
R³ is H, OH, ODMT, or OX(Y)ₚ, wherein X is a bond or a linking group; and R⁴ is one of: 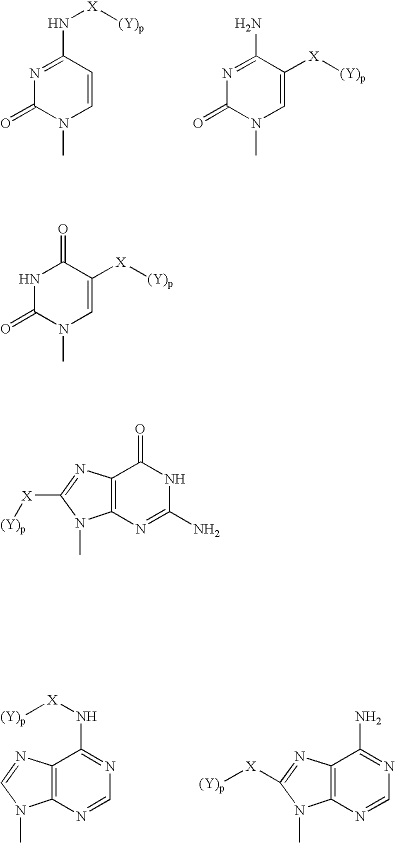
wherein:
Y is Ya or Yb: 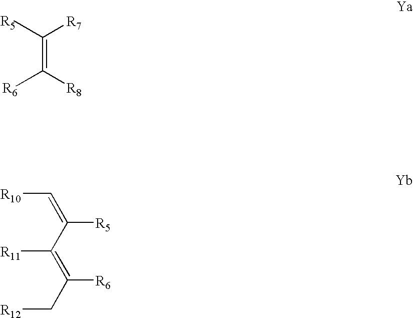
wherein R⁵ and R⁶ are independently selected from hydroxy, amino, substituted amino, carbonyl, carboxamide or N-hydroxy amide, any of which may be bound to a protecting group,
R⁷ and R⁸ are independently carboxy or cyano or together form one or more rings, wherein each ring comprises 5 to 10 atoms and one or more double bonds, in addition to the double bond of the formula Ya, wherein the atoms of each ring are optionally substituted with alkyl, hydroxy, nitro, or oxo;
R¹⁰ and R¹¹ and R¹² are independently carboxyl or cyano or together form one or more rings, wherein each ring comprises 5 to 10 atoms and one or more double bonds, in addition to the double bonds of the formula Yb, wherein the atoms of each ring are optionally substituted with alkyl, hydroxy, nitro or oxo;
X is linked to Ya through R⁷ or R⁸ or to Yb through R¹⁰ or R¹¹ or R¹²; each p in R³ and R⁴ is independently 0 to 3, and p in at least one of R³ or R⁴ is 1.

35. An oligonucleotide or polynucleotide having conjugated thereto a compound according to claim 34.

36. The compound of claim 34, wherein, in Ya, R⁷ and R⁸ together form a benzene or anthracene ring.

37. The compound of claim 34, wherein, in Yb, R¹⁰ and R¹¹ and R¹² together form a naphthalene ring.

38. The compound of claim 34, wherein Ya is of the form Ya′: 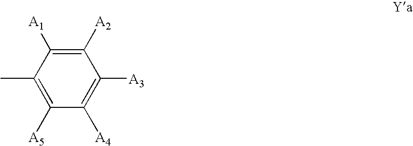
wherein A¹ to A⁵ are independently selected from hydrogen, hydroxy, amino, substituted amino, carbonyl, carboxamide and N-hydroxy amide, any of which may be bound to a protecting group, wherein at least two adjacent members of A¹ to A⁵ are not hydrogen.

39. The compound of claim 34, wherein Yb is of the form Yb′: 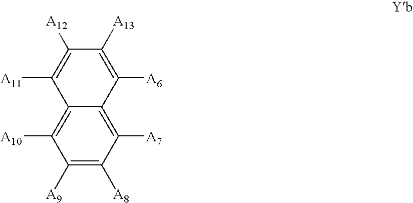
wherein
A⁶ and A⁷ are independently selected from hydroxy, amino, substituted amino, carbonyl, carboxamide and N-hydroxy amide, any of which may be bound to a protecting group,
one of A⁸ to A¹³ is linked to X, and the remaining members of A⁸ to A¹³ are independently hydrogen, hydroxy, amino, substituted amino, carbonyl, carboxamide or N-hydroxy amide.

40. The compound of claim 34, wherein, in Ya, R⁵ and R⁶ are hydroxy.

41. The compound of claim 34, wherein, in Yb, R⁵ and R⁶ are hydroxy.

42. The compound of claim 34, wherein Ya is selected from the group consisting of ortho-hydroxybenzenes (catechols), ortho-hydroxybenzohydroxamic acids, ortho-hydroxybenzoic acids, ortho-hydroxybenzamides, imidazolyl phenols, dihydroxyfumaric acids, and hydroxy pyridine aldehydes.

43. The compound of claim 34, wherein Yb is a hydroxyanthraquinone dioxime.

44. The compound of claim 34, wherein Ya is selected from the group consisting of catechol, salicylamide, N-phenylsalicylamidine, 2-(4′-imidazolyl) phenol, 1,8-dihydroxynaphthalene, dihydroxyfumaric acid, salicylaldehyde, 3-hydroxypyridine-4-aldehyde, pyridoxamine and disalicylimide.

45. The compound of claim 34, wherein Yb is 1-hydroxyanthraquinone dioxime.

46. A method of synthesizing an oligonucleotide of predetermined length, said method comprising reacting activated nucleoside monomer reagents sequentially until said oligonucleotide of predetermined length is formed, wherein at least one of said nucleoside monomer reagents is a compound of claim 34.

47. The method of claim 46, further comprising purifying said oligonucleotide by contacting a reaction mixture comprising said oligonucleotide with a boronate, to form a boronate complex therewith, and separating said complex from said mixture.

48. The method of claim 47, wherein said boronate is attached to a solid support.
